(19) United States
(12) Patent Application Publication
(10) Pub. No.: US 2022/0029108 A1
(43) Pub. Date: Jan. 27, 2022

(54) ORGANIC ELECTROLUMINESCENT COMPOUND, A PLURALITY OF HOST MATERIALS AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Chi-Sik KIM, Gyeonggi-do (KR); Soo-Yong LEE, Gyeonggi-do (KR); Seung-Hoon YOO, Gyeonggi-do (KR); Kyoung-Jin PARK, Gyeonggi-do (KR)

(21) Appl. No.: 17/347,484

(22) Filed: Jun. 14, 2021

(30) Foreign Application Priority Data
Jul. 6, 2020 (KR) 10-2020-0082709
May 3, 2021 (KR) 10-2021-0057273

(51) Int. Cl. H01L 51/00

(52) U.S. Cl. CPC H01L 51/0072; H01L 51/0055; H01L 51/0094; H01L 51/5012; H01L 51/0074; H01L 51/0067; H01L 51/0073

(57) ABSTRACT

The present disclosure relates to an organic electroluminescent compound represented by formula 1, a plurality of host materials comprising a combination of specific compounds, and an organic electroluminescent device comprising the same. By comprising the organic electroluminescent compound or the specific combination of compounds according to the present disclosure as a host material and/or an electron transport zone material, it is possible to provide an organic electroluminescent device having improved driving voltage, luminous efficiency, and/or lifetime properties, compared to the conventional organic electroluminescent device.

### TECHNICAL FIELD

The present disclosure relates to an organic electroluminescent compound, a host material comprising a combination of specific compounds, and an organic electroluminescent device comprising the same.

### BACKGROUND ART

A small molecular organic electroluminescent device (OLED) was first developed by Tang, et al., of Eastman Kodak in 1987 by using TPD/ALq3 bi-layer consisting of a light-emitting layer and a charge transport layer. Thereafter, the development of OLEDs was rapidly effected and OLEDs have been commercialized. At present, OLEDs primarily use phosphorescent materials having excellent luminous efficiency in panel implementation. An OLED having low driving voltage, high luminous efficiency and/or long lifetime is required for long time use and high resolution of a display. In addition, a green light-emitting material is recently used in OLEDs besides conventional red, green, and blue light-emitting materials. However, the phosphorescent green material has shorter lifetime than the phosphorescent red material, and thus it is required to improve the lifetime of the phosphorescent green material.

Meanwhile, Korean Patent Application Laying-Open No. 2015-0116776 discloses an organic electroluminescent device comprising a bicarbazole derivative compound and a carbazole derivative compound as a plurality of host materials. Further, Korean Patent No. 1498278 discloses a carbazole derivative compound as a single host material, a hole transport layer material, or a light-emitting auxiliary layer material. In addition. Chinese Patent Application Laying-Open No. 103467450 discloses a carbazole derivative compound as a single host material. However, development of a light-emitting material having improved performances, for example, improved driving voltage, luminous efficiency, power efficiency and/or lifetime properties, as compared with the specific compounds disclosed in the aforementioned references is still required.

### DISCLOSURE OF INVENTION

### Technical Problem

The objective of the present disclosure is to provide an organic electroluminescent compound having a new structure suitable for applying it to an organic electroluminescent device. Another objective of the present disclosure is to provide an improved organic electroluminescent material capable of providing an organic electroluminescent device having improved luminous efficiency and/or long lifetime properties. Still another objective of the present disclosure is to provide an organic electroluminescent device having improved driving voltage, luminous efficiency, and/or lifetime properties by comprising a specific combination of compounds as a host material and/or an electron transport zone material.

### Solution to Problem

The present inventors found that the above objective can be achieved by a compound represented by the following formula 1. The compound represented by the following formula 1 can be applied to an organic electroluminescent device in combination with a compound represented by the following formula 2 as a plurality of host materials.

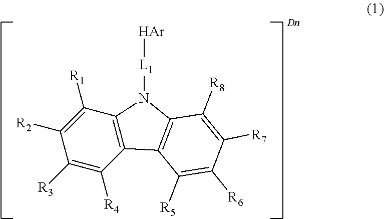

In formula 1,

HAr represents a substituted or unsubstituted (3- to 30-membered)heteroaryl;

L₁ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;

R₁ to R₅, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NR₁₃R₁₄ or —SiR₁₅R₁₆R₁₇; or may be linked to an adjacent substituent to form a ring(s),

with the proviso that at least one group of group R₅ and R₆, group R₆ and R₇, and group R₇ and R₈ of formula 1 are fused with * of the following formula 1-a to form a ring(s),

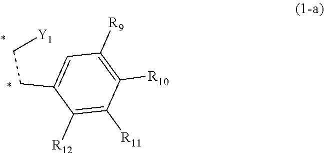

in formula 1-a,

Y₁ represents O or S,

R₉ to R₁₂, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroalyl, —NR₁₈R₁₉ or —SiR₂₀R₂₁R₂₂,

R₁₃ to R₂₂, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl; or may be linked to an adjacent substituent to form a ring(s);

Dₙ represents that n hydrogens are replaced with deuterium; and

n represents an integer of 1 to 50.

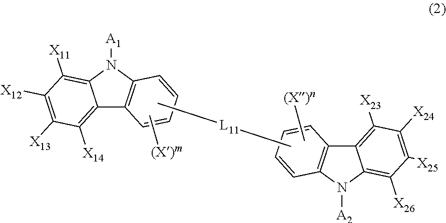

in formula 2,

A₁ and A₂, each independently, represent a substituted or unsubstituted (C6-C30)aryl, or a substituted or unsubstituted (3- to 30-membered)heteroaryl;

L₁₁ represents a single bond, or a substituted or unsubstituted (C6-C30)arylene.

X′, X″, X₁₁, to X₁₄, and X₂₃ to X₂₆, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C30)aryl, a substituted or unsubstituted (3- to 30-membered)heteroaryl, —NR₂₃R₂₄ or —SiR₂₅R₂₆R₂₇; or may be linked to an adjacent substituent to form a ring(s);

R₂₃ to R₂₇, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl; or may be linked to an adjacent substituent to form a ring(s);

m and n, each independently, represent an integer of 1 to 3; and

if m and n are an integer of 2 or more, each of X′ and each of X″ may be the same or different.

### Advantageous Effects of Invention

The organic electroluminescent compound according to the present disclosure exhibits performances suitable for using it in an organic electroluminescent device. In addition, by comprising a specific combination of compounds according to the present disclosure as a host material and/or an electron transport zone material, an organic electroluminescent device having high luminous efficiency and/or long lifetime properties compared to conventional organic electroluminescent devices is provided. For example, by comprising the compound according to the present disclosure, a green light-emitting or blue light-emitting organic electroluminescent device having improved lifetime property can be provided.

### MODE FOR THE INVENTION

Hereinafter, the present disclosure will be described in detail. However, the following description is intended to explain the present disclosure and is not meant in any way to restrict the scope of the present disclosure.

The term “an organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

The term “a plurality of organic electroluminescent materials” in the present disclosure means an organic electroluminescent material comprising a combination of at least two compounds, which may be comprised in any organic layer constituting an organic electroluminescent device. It may mean both a material before being comprised in an organic electroluminescent device (for example, before vapor deposition) and a material after being comprised in an organic electroluminescent device (for example, after vapor deposition), For example, a plurality of organic electroluminescent materials of the present disclosure may be a combination of at least two compounds, which may be comprised in at least one layer of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. The at least two compounds may be comprised in the same layer or different layers by means of the methods used in the art, and may be mixture-evaporated or co-evaporated, or may be individually evaporated.

The term “a plurality of host materials” in the present disclosure means a host material comprising a combination of at least two host materials, which may be comprised in any light-emitting layer constituting an organic electroluminescent device. It may mean both a material before being comprised in an organic electroluminescent device (for example, before vapor deposition) and a material after being comprised in an organic electroluminescent device (for example, after vapor deposition). For example, the plurality of host materials of the present disclosure is a combination of at least two host materials, and may selectively further comprise conventional materials comprised in an organic electroluminescent material. At least two compounds comprised in the plurality of host materials of the present disclosure may be comprised together in one light-emitting layer or may respectively be comprised in different light-emitting layers. For example, the at least two host materials may be mixture-evaporated or co-evaporated, or may be individually evaporated.

The organic electroluminescent material of the present disclosure may comprise at least one compound represented by formula 1. The compound represented by formula 1 may be comprised in a light-emitting layer, but is not limited thereto. When comprised in a light-emitting layer, the compound represented by formula 1 may be comprised as a host material. Further, the compound represented by formula 1 may be comprised in an electron transport zone. In addition, the compound represented by formula 1 may be comprised in an electron buffer layer, but is not limited thereto.

Herein, the term “(C1-C30)alkyl(ene)” is meant to be a linear or branched alkylene) having 1 to 30 carbon atoms constituting the chain, in which the number of carbon atoms is preferably 1 to 10, and more preferably 1 to 6. The above alkyl may include methyl, ethyl, n-propyl, isopropyl, n-butyl, isobutyl, Cert-butyl, sec-butyl, etc. The term “(C3-C30)cycloakyl(ene)” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. The term “(3- to 7-membered)heterocycloalkyl” is meant to be a cycloalkyl having 3 to 7 ring backbone atoms, and including at least one heteroatom selected from the group consisting of B, N, O, S, Si, and P, and preferably from the group consisting of O, S, and N. The above heterocycloalkyl may include tetrahydrofuran, pyrrolidine, thiolan, tetrahydropyran, etc. The term “(C6-C30)aryl(ene)” is meant to be a monocyclic or fused ring radical derived from an aromatic hydrocarbon having 6 to 30 ring backbone carbon atoms. The above aryl(ene) may be partially saturated, and may comprise a Spiro structure. The above aryl may include phenyl, biphenyl, terphenyl, naphthyl, binaphthyl, phenylnaphthyl, naphthylphenyl, fluorenyl, phenylfluorenyl, diphenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-benzofluorene]yl, azulenyl, tetramethyldihydrophenanthrenyl, etc. Specifically, the above aryl may include phenyl, 1-naphthyl, 2-naphthyl, 1-anthryl, 2-anthryl, 9-anthryl, benzanthryl, 1-phenanthyl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl. 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl. 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl. 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl. 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl(ene)” is meant to be an aryl or an arylene having 3 to 30 ring backbone atoms, and including at least one, preferably 1 to 4 heteroatoms selected from the group consisting of B, N, O, S, Si, and P. The above heteroaryl(ene) may be a monocyclic ring, or a fused ring condensed with at least one benzene ring; may be partially saturated; may be one formed by linking at least one heteroaryl or aryl group to a heteroaryl group via a single bond(s); and may comprise a Spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, and pyridazinyl, and a fused ring-type heteroaryl such as benzofuranyl, benzothiophenyl, isobenzofuranyl, dibenzofuranyl, dibenzothiophenyl, dibenzoselenophenyl, naphthobenzofuranyl, naphthobenzothiophenyl, benzofuroquinolinyl, benzofuroquinazolinyl, benzofuronaphthyridinyl, benzofuropyrimidinyl, naphthofuropyrimidinyl, benzothienoquinolinyl, benzothienoquinazolinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzirnidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, benzocarbazolyl, dibenzocarbazolyl, phenoxazinyl, phenanthridinyl, benzodioxolyl, dihydroacridinyl, benzotriazolphenazinyl, irnidazopyridinyl, chromenoquinazolinyl, thiochromenoquinazolinyl, dimethylbenzoperimidinyl, indolocarbazolyl, indenocarbazolyl, etc. More specifically, the above heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoguinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl 3 indolyl, 2-test-butyl-1-indolyl, 4-Cert-butyl-1-indolyl, 2-Cert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl; 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl; 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Furthermore, “halogen” includes F, Cl, Br, and I.

In addition, “ortho (o-),” “meta (m-),” and “para (p-)” are prefixes, which represent the relative positions of substituents respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

Herein, “substituted” in the expression “substituted or unsubstituted” means that a hydrogen atom in a certain functional group is replaced with another atom or another functional group, i.e., a substituent, and also includes that the hydrogen atom is replaced with a group formed by a linkage of two or more substituents of the above substituents. For example, the “group formed by a linkage of two or more substituents” may be pyridine-triazine. That is, pyridine-triazine may be interpreted as a heteroaryl substituent, or as substituents in which two heteroaryl substituents are linked. Herein, the substituent(s) of the substituted alkyl(ene), the substituted alkenyl, the substituted alkynyl, the substituted aryl(ene), the substituted heteroaryl(ene), the substituted cycloalkyl(ene), the substituted cycloalkenyl, and the substituted heterocycloalkyl, each independently, are at least one selected from the group consisting of deuterium; a halogen; a cyano; a carboxyl; a nitro; a hydroxyl; a phosphineoxide; a (C1-C30)alkyl; a halo(C1-C30)alkyl; a (C2-C30)alkenyl: a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of deuterium and a (C6-C30)aryl(s); a (C6-C30)aryl unsubstituted or substituted with at least one of deuterium and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C2-C30)alkenylamino: a mono- or di-(C6-C30)arylamino unsubstituted or substituted with a (C1-C30)alkyl(s); a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino: a (C6-C30)aryl(3- to 30-membered)heteroarylamino; a (C1-C30)alkylcarbonyl; a (C1-C30)alkoxycarbonyl, a (C6-C30)arylcarbonyl; a (C6-C30)arylphosphine; a di(C6-C30)arylboronyl: a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituent(s), each independently, are at least one selected from the group consisting of deuterium; a (C1-C20)alkyl; a (5- to 25-membered)heteroaryl unsubstituted or substituted with at least one of deuterium and a (C6-C25)aryl(s); a (C5-C25)aryl unsubstituted or substituted with at least one of deuterium and a (5- to 25-membered)heteroaryl(s); and tri(C6-C25)arylsilyl. According to another embodiment of the present disclosure, the substituent(s); each independently, are at least one selected from the group consisting of deuterium; a (C1-C10)alkyl; a (5- to 20-membered)heteroaryl unsubstituted or substituted with at least one of deuterium and a (C6-C18)aryl(s): a (C6-C18)aryl unsubstituted or substituted with at least one of deuterium and a (5- to 20-membered)heteroaryl(s), and a tri(C6-C18)arylsilyl. For example, the substituent(s) may be at least one selected from the group consisting of deuterium; a methyl; a phenyl; a phenyl substituted with at least one deuterium; a phenyl substituted with a carbazolyl(s); a naphthyl; a biphenyl; a pyridyl unsubstituted or substituted with a phenyl(s); a dibenzofuranyl; a dibenzothiophenyl; a carbazolyl substituted with a phenyl(s); and a triphenylsilyl.

Herein, a ring formed by a linkage of adjacent substituents means that at least two adjacent substituents are linked to or fused with each other to form a substituted or unsubstituted, mono- or polycyclic, (3- to 30-membered) alicyclic or aromatic ring, or the combination thereof. Preferably, the ring may be a substituted or unsubstituted, mono- or polycyclic, (3- to 26-membered) alicyclic or aromatic ring, or the combination thereof. More preferably, the ring may be a mono- or polycyclic, (5- to 25-membered) aromatic ring unsubstituted or substituted with at least one of a (C6-C18)aryl(s) and a (3- to 20-membered)heteroaryl(s), In addition, the formed ring may contain at least one heteroatom selected from B; N, O, S, Si, and P, preferably at least one heteroatom selected from N, O, and S. For example, the ring may be a benzene ring; an indole ring substituted with at least one of a phenyl(s), a biphenyl(s), a naphthyl(s), a naphthylphenyl(s), a phenylnaphthyl(s); a terphenyl(s), a triphenylenyl(s), a phenylpyridyl(s), and phenylpyrimidinyl(s); a spiro[indene-xanten] ring unsubstituted or substituted with a phenylcarbazolyl(s); a xanten ring unsubstituted or substituted with a phenylcarbazolyl(s), etc.

In the present disclosure, heteroaryl, heteroarylene, and heterocycloalkyl may, each independently, contain at least one heteroatom selected from B, N, O, S, Si, and P. In addition, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (5- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, substituted or unsubstituted (C1-C30)alkyl(3- to 30-membered)heteroarylamino, substituted or unsubstituted (C2-C30)alkenyl(C6-C30)arylamino, a substituted or unsubstituted (C2-C30)alkenyl(3- to 30-membered)heteroarylamino, and a substituted or unsubstituted (C6-C30)aryl(3- to 30-membered)heteroarylamino.

A plurality of host materials of the present disclosure comprises a first host material and a second host material, in which the first host material comprises the compound represented by formula 1 and the second host material comprises the compound represented by formula 2. According to one embodiment of the present disclosure, the compound represented by formula 1 and the compound represented by formula 2 are different from each other.

In formula 1, HAr represents a substituted or unsubstituted (3- to 30-membered)heteroaryl. According to one embodiment of the present disclosure, HAr represents a substituted or unsubstituted, nitrogen-containing (5- to 25-membered)heteroaryl. According to another embodiment of the present disclosure, HAr represents a nitrogen-containing (5- to 20-membered)heteroaryl substituted with at least one of deuterium, a (C6-C30)aryl(s), and a (3- to 30-membered)heteroaryl(s). Specifically, HAr may be a substituted or unsubstituted triazinyl, a substituted or unsubstituted pyridyl, a substituted or unsubstituted pyrimidinyl, a substituted or unsubstituted quinazolinyl, a substituted or unsubstituted benzoquinazolinyl, a substituted or unsubstituted quinoxalinyl, a substituted or unsubstituted benzoquinoxalinyl, a substituted or unsubstituted quinolyl, a substituted or unsubstituted benzoquinolyl, a substituted or unsubstituted isoquinolyl, a substituted or unsubstituted benzoisoquinolyl, a substituted or unsubstituted triazolyl, a substituted or unsubstituted pyrazolyl, a substituted or unsubstituted naphthyridinyl, a substituted or unsubstituted triazanaphthyl, or a substituted or unsubstituted benzothienopyrimidinyl. For example, HAr may be a substituted triazinyl, a substituted pyrimidinyl, a substituted quinazolinyl, or a substituted quinoxalinyl, in which the substituent(s) of the substituted triazinyl, the substituted pyrimidinyl, the substituted quinazolinyl, and the substituted quinoxalinyl, each independently, may be at least one of a phenyl unsubstituted or substituted with deuterium and/or a carbazolyl(s), a biphenyl, a dibenzofuranyl, a dibenzothiophenyl, and a carbazolyl substituted with a phenyl(s).

In formula 1, L₁ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene. According to one embodiment of the present disclosure, L₁ represents a single bond, a substituted or unsubstituted (C6-C25)arylene, or a substituted or unsubstituted (5- to 25-membered)heteroarylene. According to another embodiment of the present disclosure, L₁ represents a single bond, a (C6-C18)arylene unsubstituted or substituted with at least one deuterium, or a (5- to 20-membered)heteroarylene unsubstituted or substituted with at least one deuterium. For example, L₁ may be a single bond, a phenylene unsubstituted or substituted with at least one deuterium, a biphenylene unsubstituted or substituted with at least one deuterium, or a dibenzofuranylene unsubstituted or substituted with at least one deuterium. Specifically, L₁ may be a single bond or a dibenzofuranylene unsubstituted or substituted with at least one deuterium, or may be represented by any one selected from the group consisting of the following:

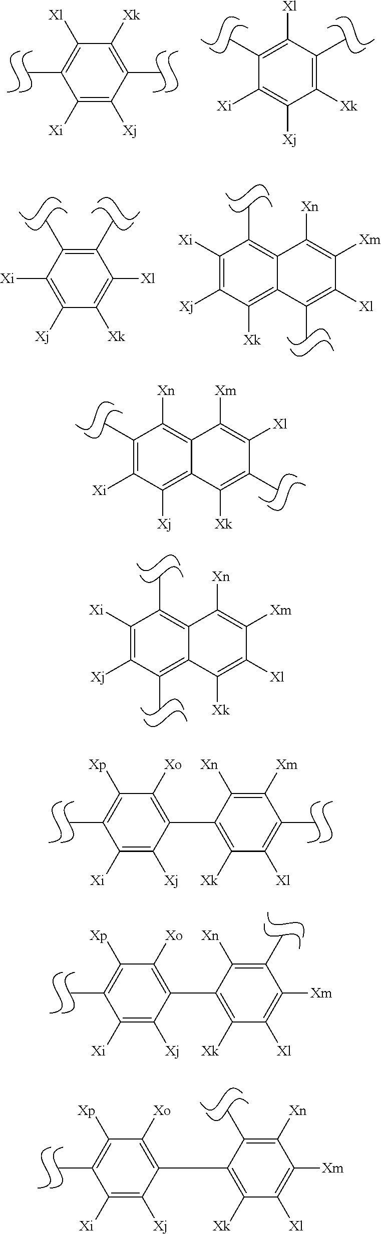

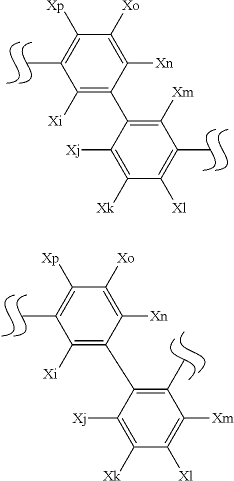

In the formulas above, Xᵢ to Xₚ, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C30)aryl, a substituted or unsubstituted (3- to 30-membered)heteroaryl, —NR₂₈R₂₉ or —SiR₃₀R₃₁R₃₂; or may be linked to an adjacent substituent to form a ring(s). For example, Xᵢ to Xₚ, each independently, may be hydrogen or deuterium.

In formula 1, R₁ to R₈, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NR₁₃R₁₄ or —SiR₁₅R₁₈R₁₇; or may be linked to an adjacent substituent to form a ring(s).

According to one embodiment of the present disclosure, R₁ to R₄, each independently, represent hydrogen, deuterium, a substituted or unsubstituted (C6-C25)aryl, or a substituted or unsubstituted (5- to 25-membered)heteroaryl. According to another embodiment of the present disclosure, R₁ to R₄, each independently, represent hydrogen, deuterium, a (C6-C18)aryl unsubstituted or substituted with deuterium, or a (5- to 20-membered)heteroaryl unsubstituted or substituted with deuterium. For example, R₁ to R₄, each independently, may be hydrogen, deuterium, a phenyl unsubstituted or substituted with at least one deuterium, a biphenyl unsubstituted or substituted with at least one deuterium, a dibenzofuranyl unsubstituted or substituted with at least one deuterium, or a carbazolyl unsubstituted or substituted with at least one deuterium.

According to one embodiment of the present disclosure, R₅ to R₈, each independently, represent hydrogen or deuterium; or may be linked to an adjacent substituent to form a ring(s). For example, R₅ to R₈, each independently, may be hydrogen or deuterium; or may be linked to an adjacent substituent to form a benzene ring unsubstituted or substituted with at least one deuterium, a benzofuran ring unsubstituted or substituted with at least one deuterium, or a benzothiophene ring unsubstituted or substituted with at least one deuterium.

At least one group of group R₅ and R₆, group R₆ and R₇, and group R₇ and R₅ of formula 1 are fused with * of the following formula 1-a to form a ring(s).

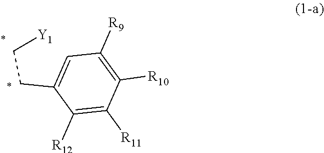

In formula 1-a, Y₁ represents O or S.

In formula 1-a, R₉ to R₁₂, each independently, represent hydrogen, deuterium, a halogen, a cyano a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NR₁₈R₁₉ or —SiR₂₀R₂₁R₂₂. According to one embodiment of the present disclosure, R₉ to R₁₂, each independently, represent hydrogen, deuterium, or a substituted or unsubstituted (C6-C25)aryl, According to another embodiment of the present disclosure, R₉ to R₁₂, each independently, represent hydrogen, deuterium, or a (C6-C18)aryl unsubstituted or substituted with deuterium. For example, R₉ to R₁₂, each independently, may be hydrogen, deuterium, or a phenyl unsubstituted or substituted with at least one deuterium.

In formula 1, Dₙ represents that n hydrogens are replaced with deuterium; and n, each independently, represents an integer of 1 to 50. According to one embodiment of the present disclosure, n, each independently, represents an integer of 5 to 50. According to another embodiment of the present disclosure, n, each independently, represents an integer of 6 to 50. According to still another embodiment of the present disclosure, n, each independently, represents an integer of 7 to 50, When being deuterated to the number of the lower limit or more, the bond dissociation energy related to deuteration may increase to exhibit improved lifetime property. The upper limit of n is determined by the number of hydrogens capable of being substituted in each compound.

R₁₃ to R₂₂ and R₂₈ to R₃₂, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl; or may be linked to an adjacent substituent to form a ring(s).

According to one embodiment of the present disclosure, the formula 1 may be represented by at least one of the following formulas 1-1 to 1-18.

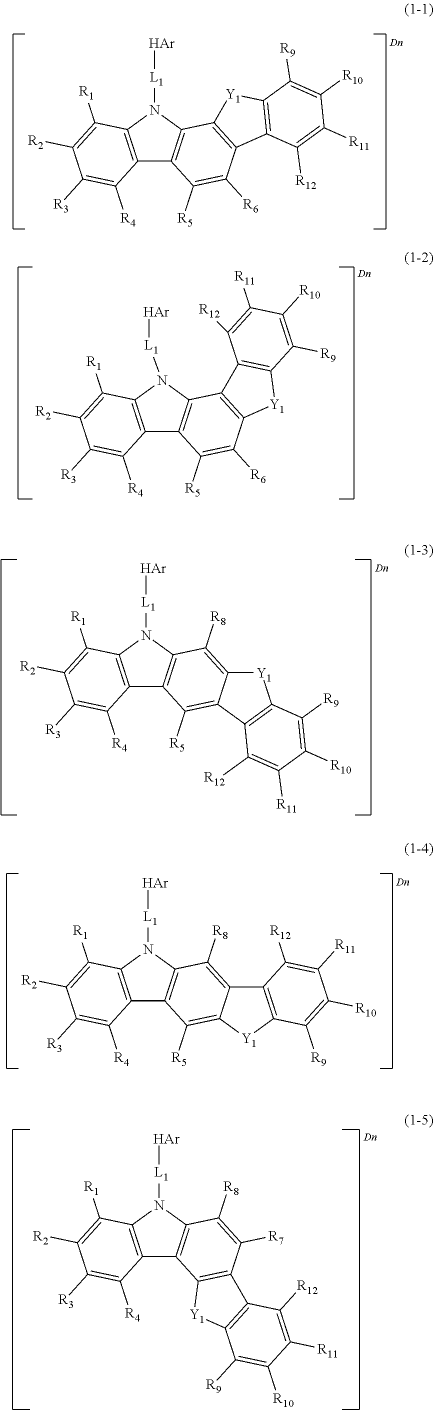

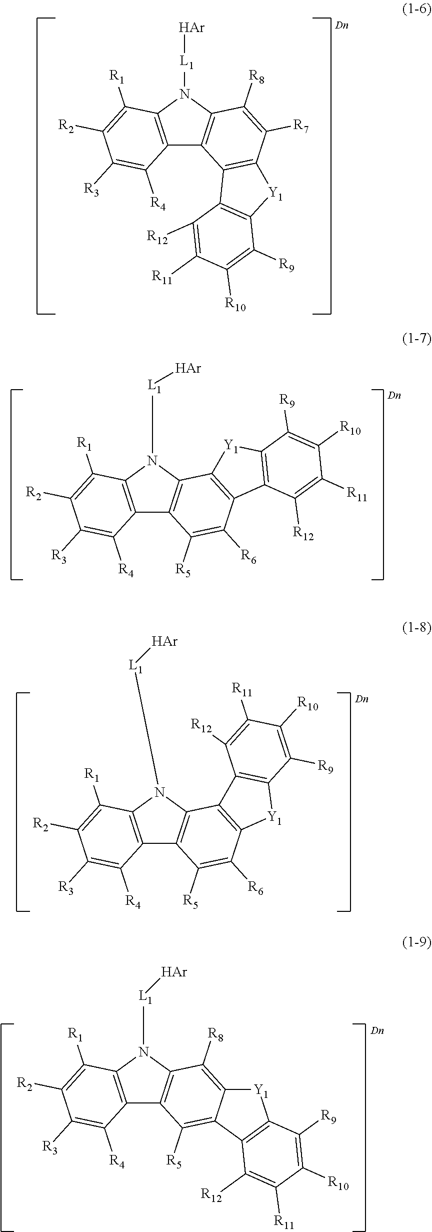

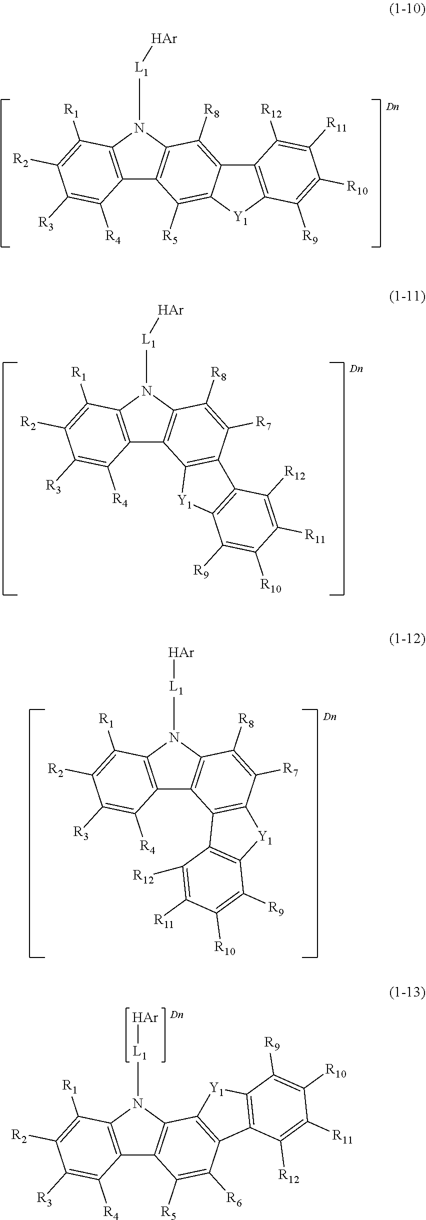

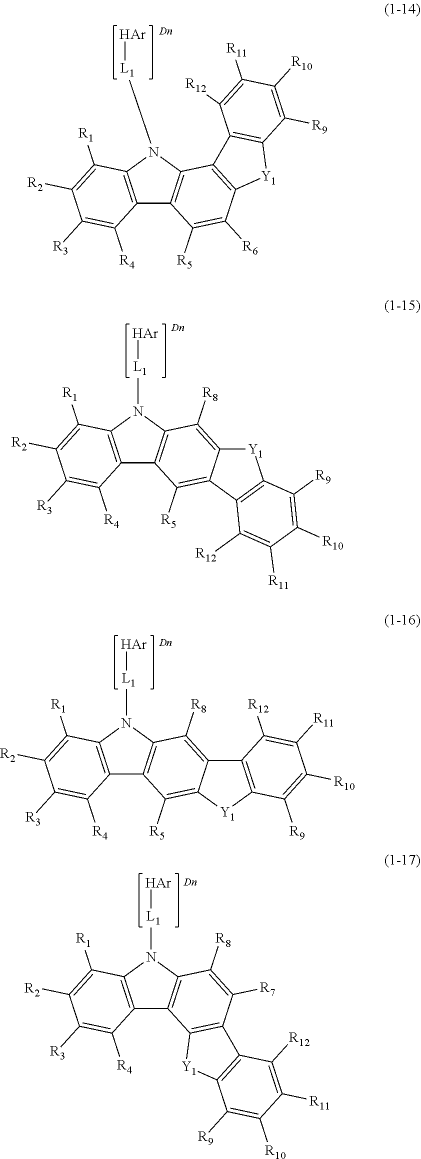

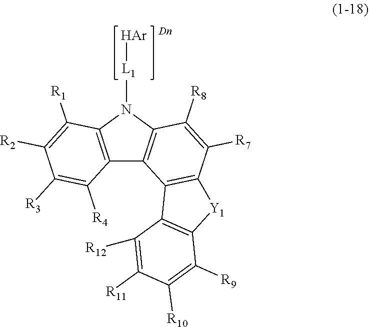

In formulas 1-1 to 1-18, R₁ to R₁₂, Y₁, L₁, HAr, and Dry are as defined in formula 1.

In formula 2, A₁ and A₂, each independently, represent a substituted or unsubstituted (C6-C30)aryl, or a substituted or unsubstituted (3- to 30-membered)heteroaryl. According to one embodiment of the present disclosure, A₁ and A₂, each independently, represent a substituted or unsubstituted (C6-C25)aryl, or a substituted or unsubstituted (5- to 25-membered)heteroaryl. According to another embodiment of the present disclosure, A₁ and A₂, each independently, represent a (C6-C18)aryl unsubstituted or substituted with at least one of deuterium, a (C1-C30)alkyl(s), a (C6-C30)aryl(s), a (3- to 30-membered)heteroaryl(s), and a tri(C6-C30)arylsilyl(s). Specifically, A₁ and A₂, each independently, may be a substituted or unsubstituted phenyl, a substituted or unsubstituted biphenyl, a substituted or unsubstituted terphenyl, a substituted or unsubstituted naphthyl, a substituted or unsubstituted fluorenyl, a substituted or unsubstituted benzofluorenyl, a substituted or unsubstituted triphenylenyl, a substituted or unsubstituted fluoranthenyl, a substituted or unsubstituted phenanthrenyl, a substituted or unsubstituted dibenzofuranyl, a substituted or unsubstituted carbazolyl, or a substituted or unsubstituted dibenzothiophenyl. For example, A₁ and A₂, each independently, may be a substituted or unsubstituted phenyl, a naphthyl, a biphenyl, a naphthylphenyl, a dimethylfuorenyl, a diphenylfluorenyl, or a dimethylbenzofluorenyl, in which the substituents of the substituted phenyl may be at least one of a methyl(s), a naphthyl(s), a triphenylsilyl(s), and a pyridyl(s) unsubstituted or substituted with a phenyl(s).

In formula 2, L₁₁ represents a single bond, or a substituted or unsubstituted (C6-C30)arylene. According to one embodiment of the present disclosure, L₁₁ represents a single bond, or a substituted or unsubstituted (C6-C25)arylene. According to another embodiment of the present disclosure, L₁₁ represents a single bond, or an unsubstituted (C6-C18)arylene. For example, L₁₁ may be a single bond, a phenylene, a naphthylene, or a biphenylene.

In formula 2, X″, X₁₁ to X₁₄, and X₂₃ to X₂₆, each independently; represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl; a substituted or unsubstituted (C6-C30)aryl, a substituted or unsubstituted (3- to 30-membered)heteroaryl, —NR₂₃R₂₄ or —SiR₂₅R₂₆R₂₇; or may be linked to an adjacent substituent to form a ring(s). According to one embodiment of the present disclosure, X′, X″, X₁₁ to X₁₄, and X₂₃ to X₂₆, each independently, represent hydrogen, or a substituted or unsubstituted (5- to 25-membered)heteroaryl; or may be linked to an adjacent substituent to form a ring(s). According to another embodiment of the present disclosure. X′, X″, X₁₁ to X₁₄, and X₂₃ to X₂₆, each independently, represent hydrogen, or an unsubstituted (5- to 20-membered)heteroaryl; or may be linked to an adjacent substituent to form a ring(s). For example, X′ and X″ may be hydrogen; and X₁₁ to X₁₄ and X₂₃ to X₂₆, each independently, may be hydrogen, a dibenzothiophenyl, or a dibenzofuranyl, or may be linked to an adjacent substituent to form a benzene ring(s).

R₂₃ to R₂₇, each independently, represent hydrogen, deuterium, a halogen, a cyano, a substituted or unsubstituted (C1-C30)alkyl; a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl; or may be linked to an adjacent substituent to form a ring(s).

In formula 2, m and n, each independently, represent an integer of 1 to 3, where if m and n are an integer of 2 or more, each of X′ and each of X″ may be the same or different. For example, m and n, each independently, may be 1.

According to one embodiment of the present disclosure, the formula 2 may be represented by at least one of the following formulas 2-1 to 2-8.

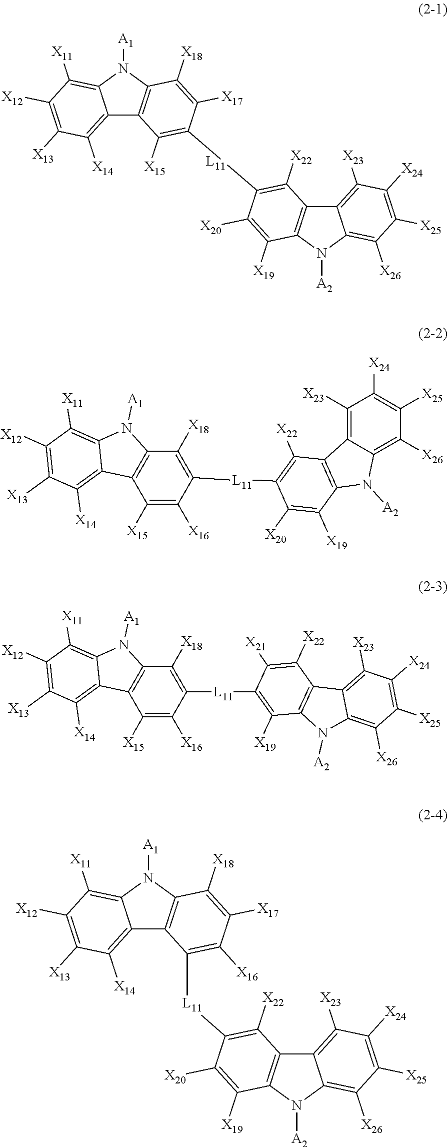

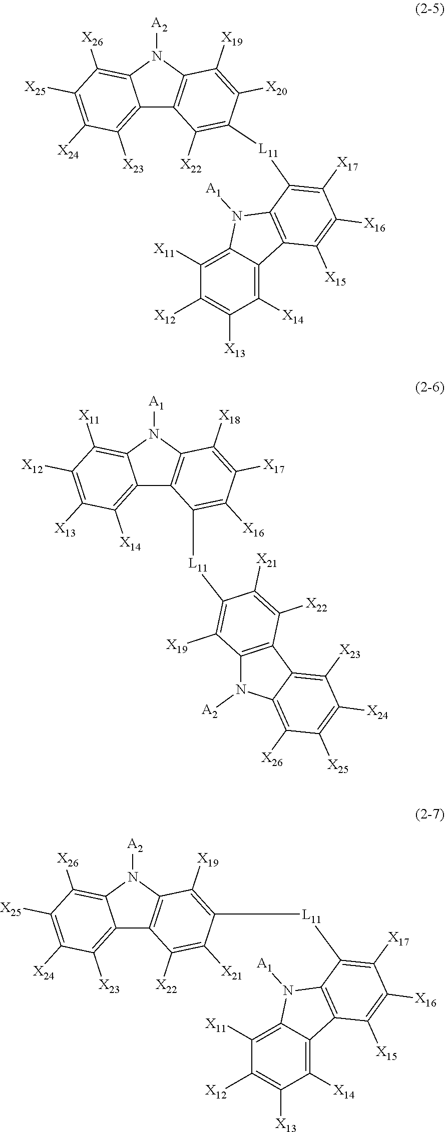

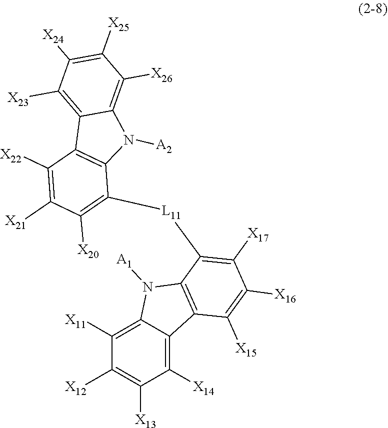

In formulas 2-1 to 2-8. A₁, A₂, X₁₁ to X₁₄, and X₂₃ to X₂₆ are as defined in formula 2; and X₁₅ to X₂₂, each independently; are the same as the definition of X′ in formula 2.

The compound represented by formula 1 may be exemplified as the following compounds, but is not limited thereto.

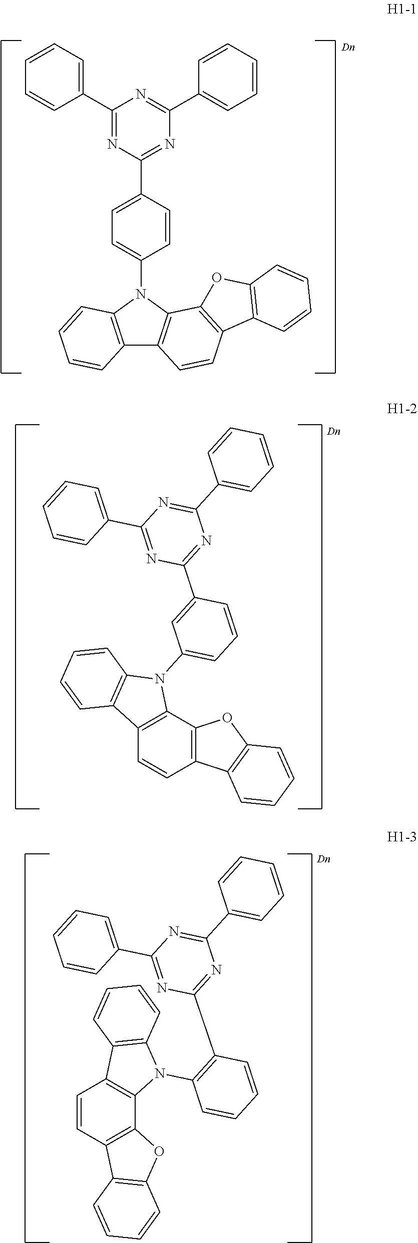

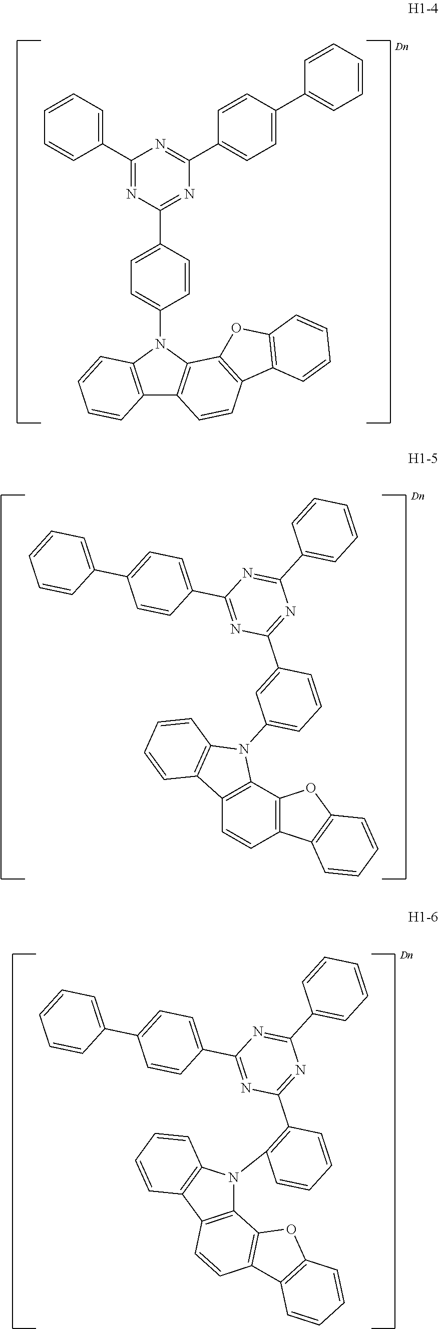

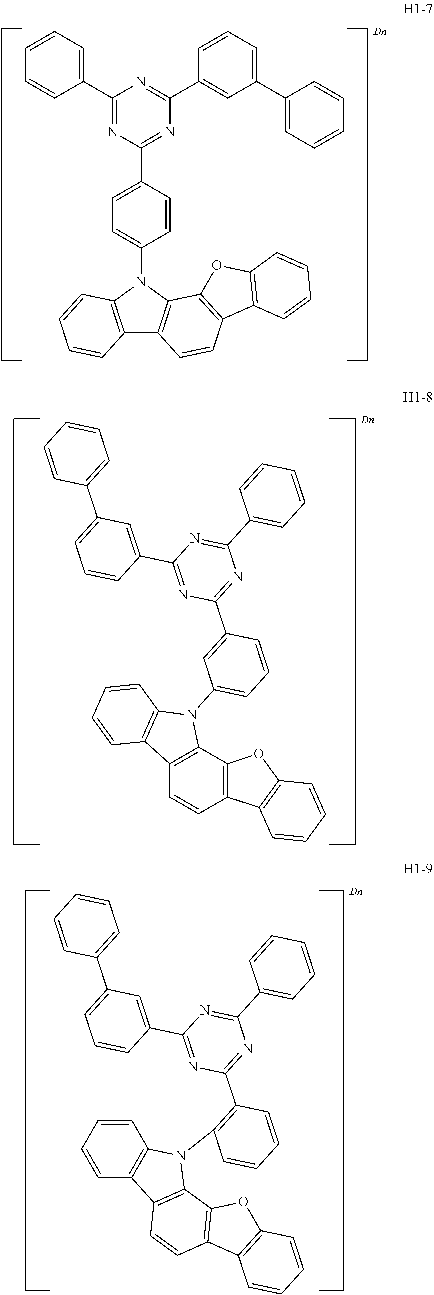

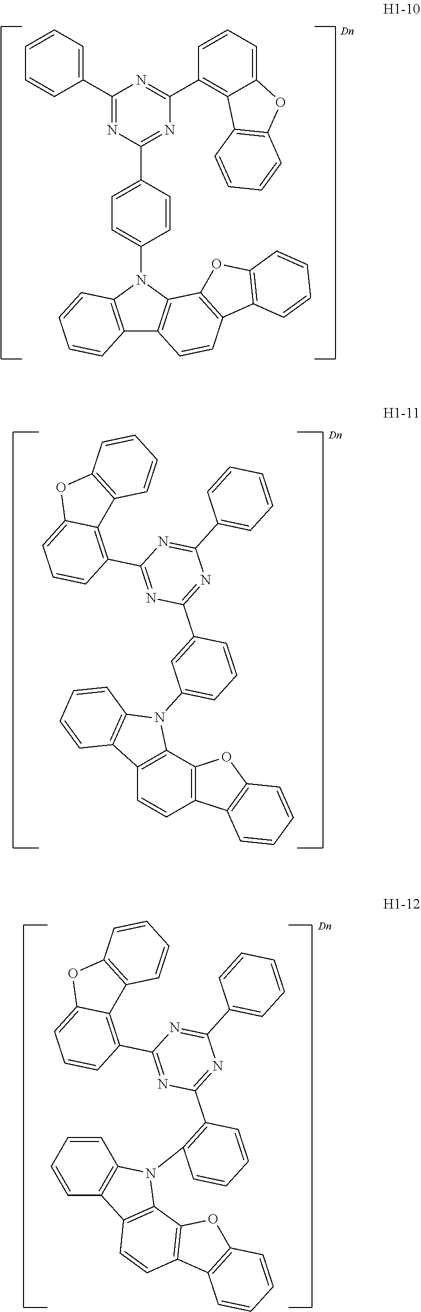

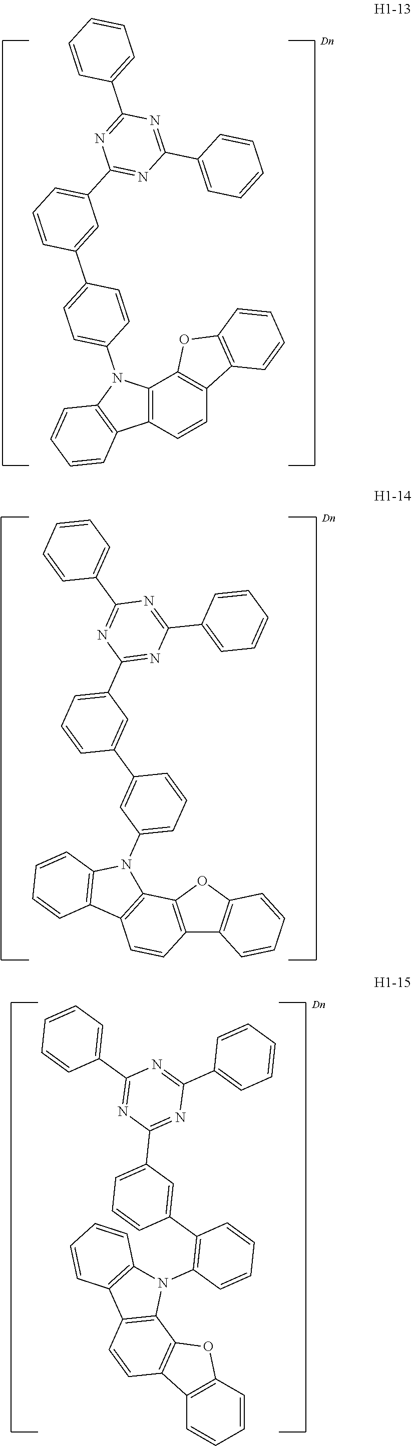

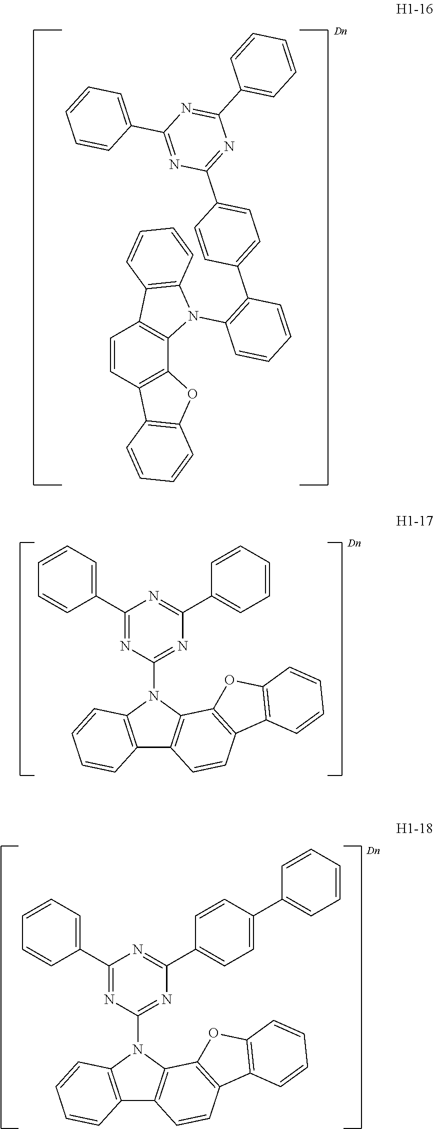

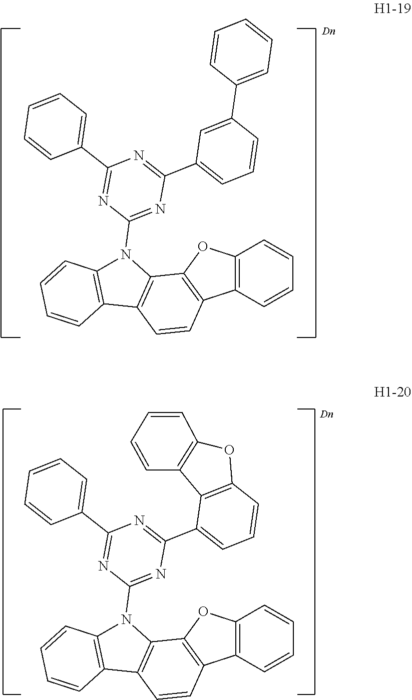

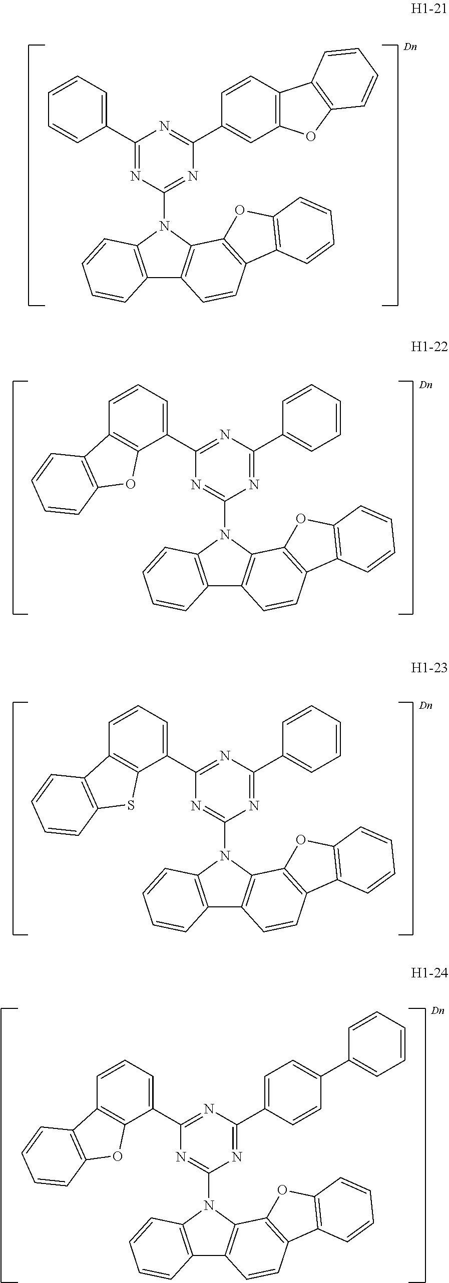

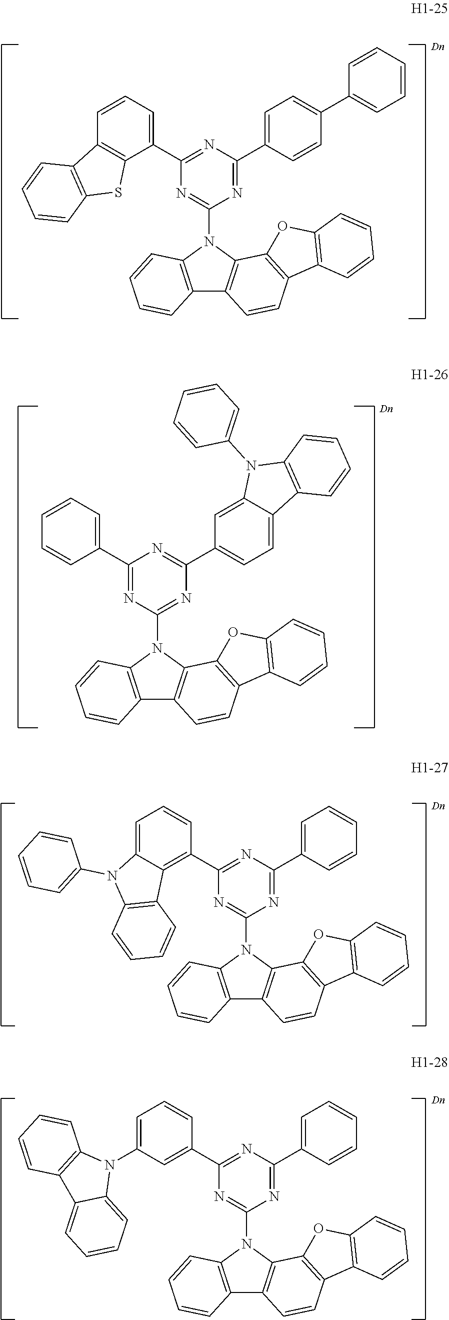

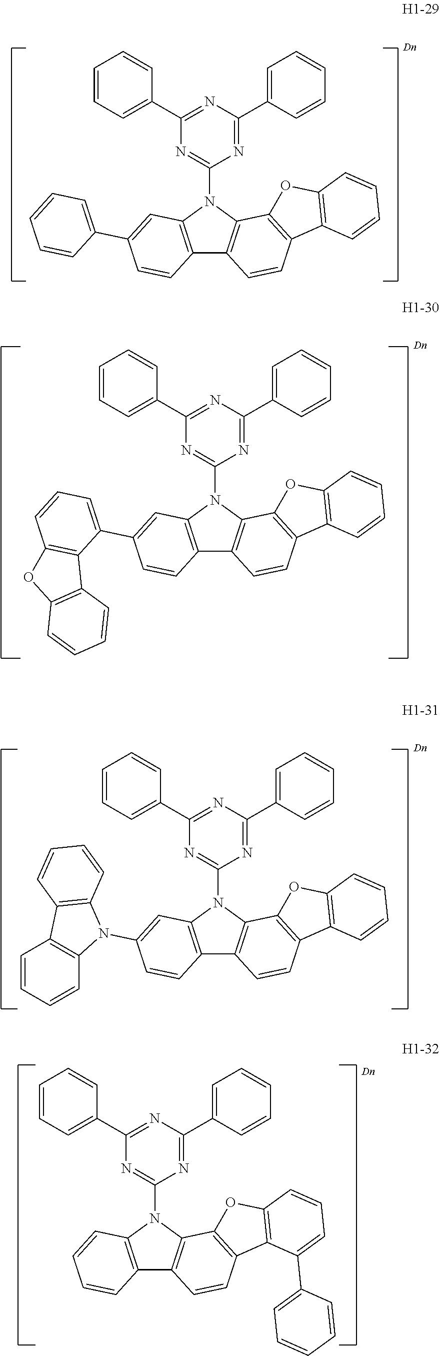

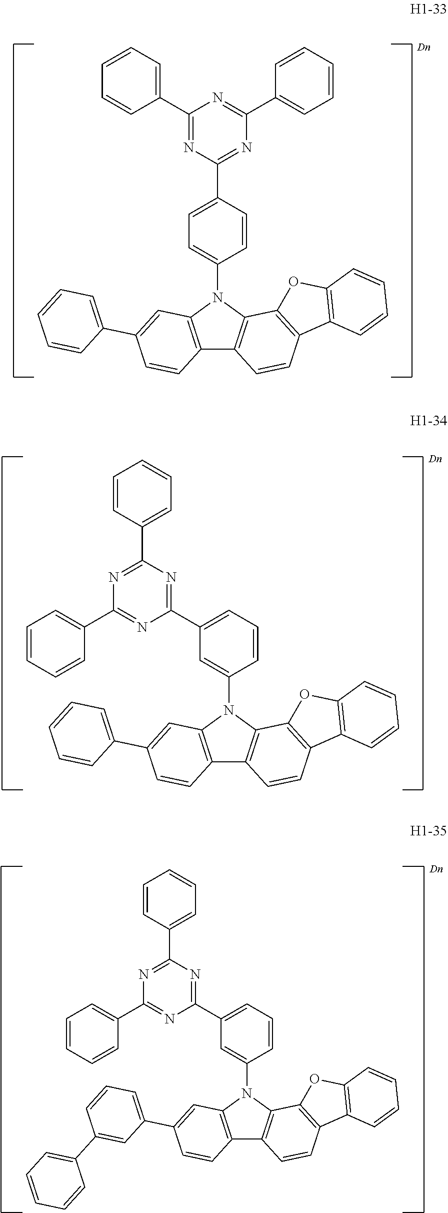

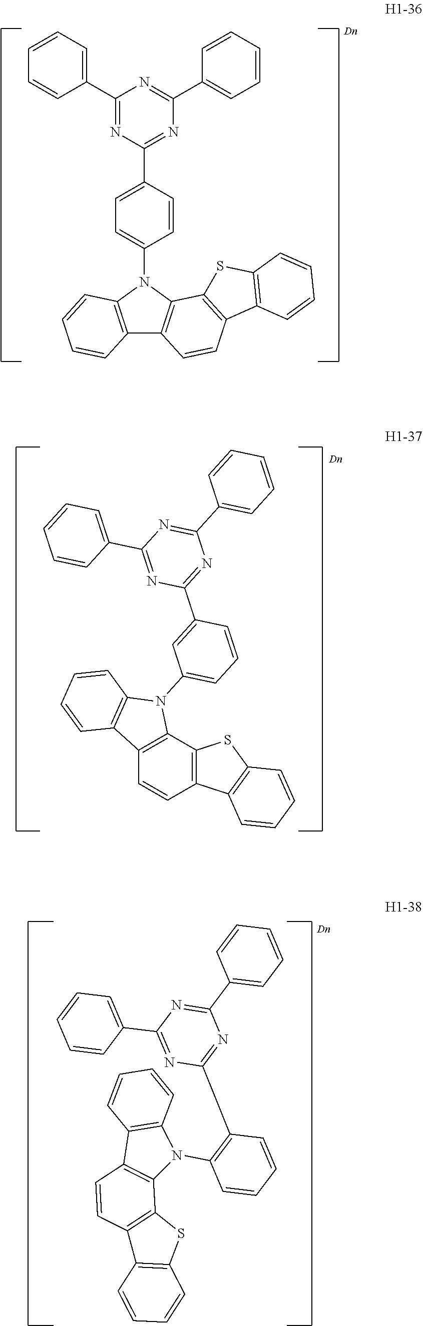

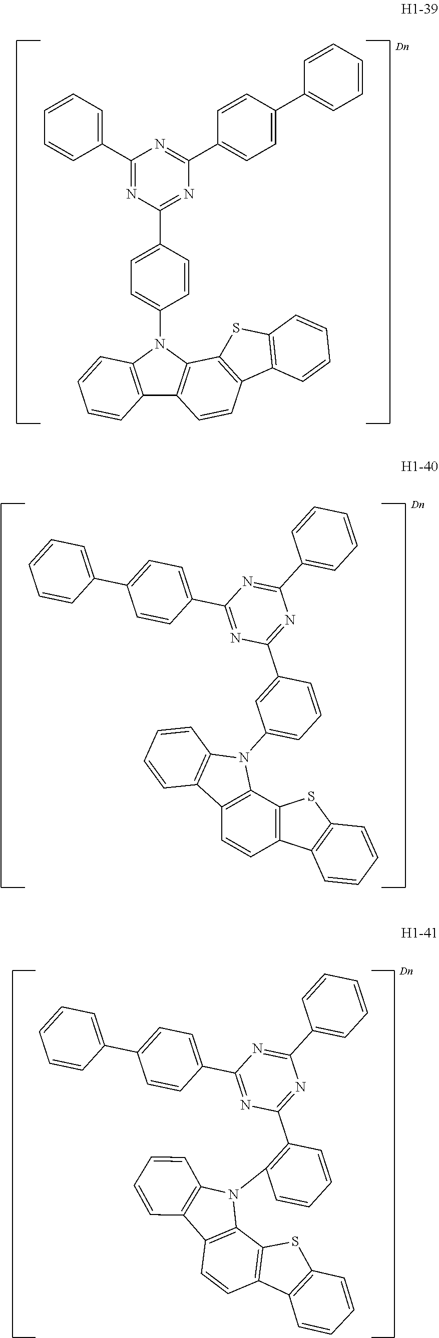

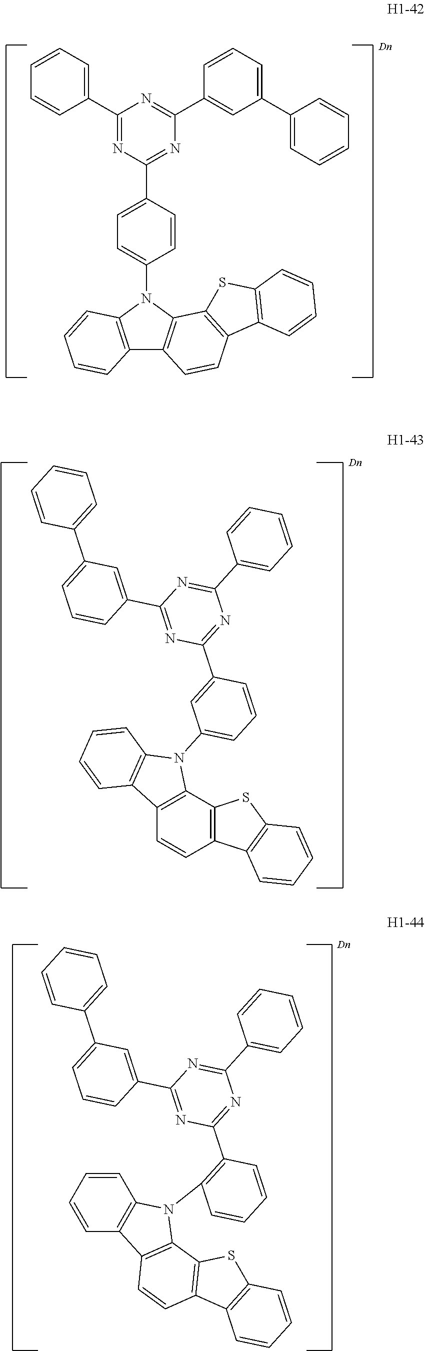

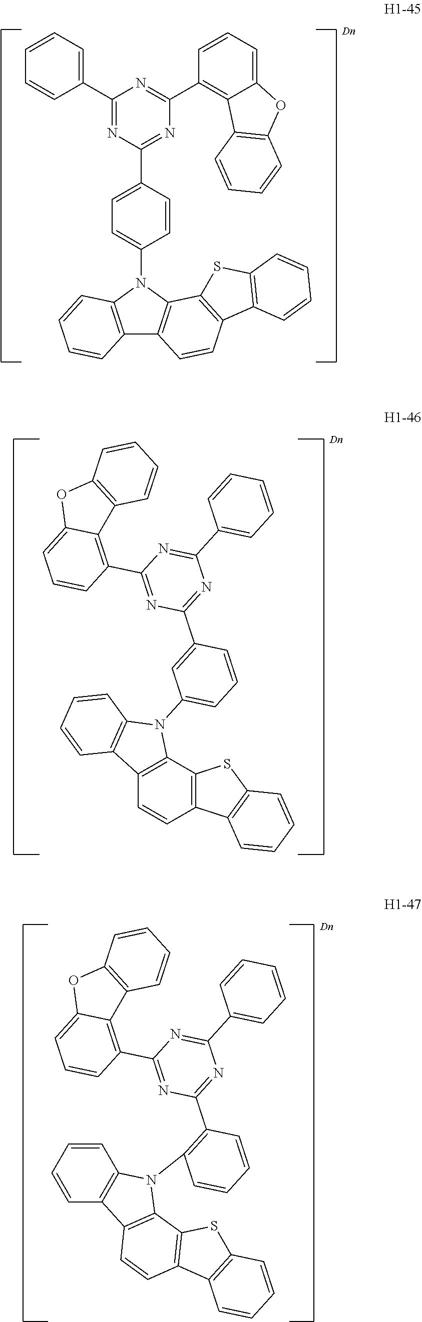

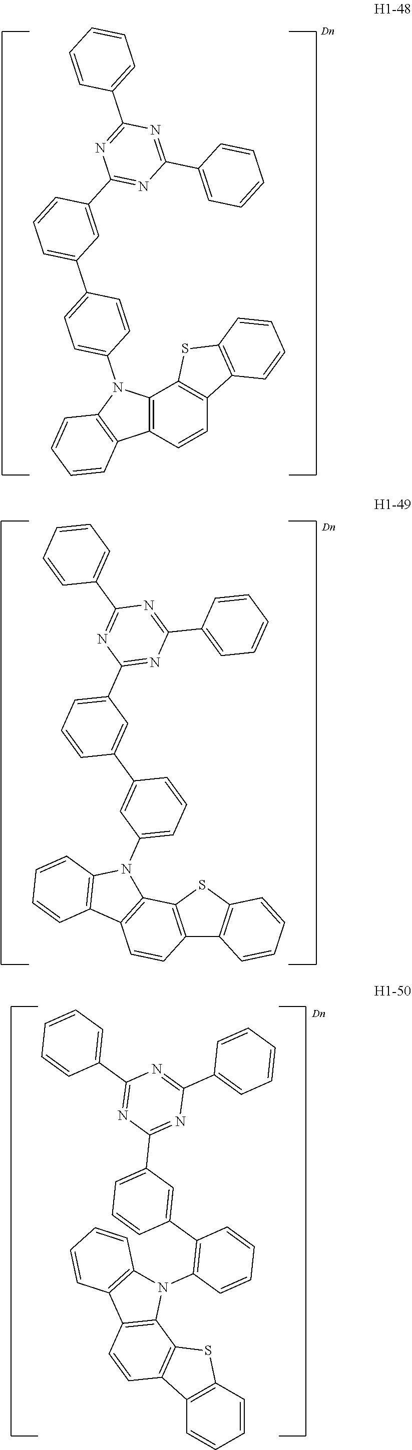

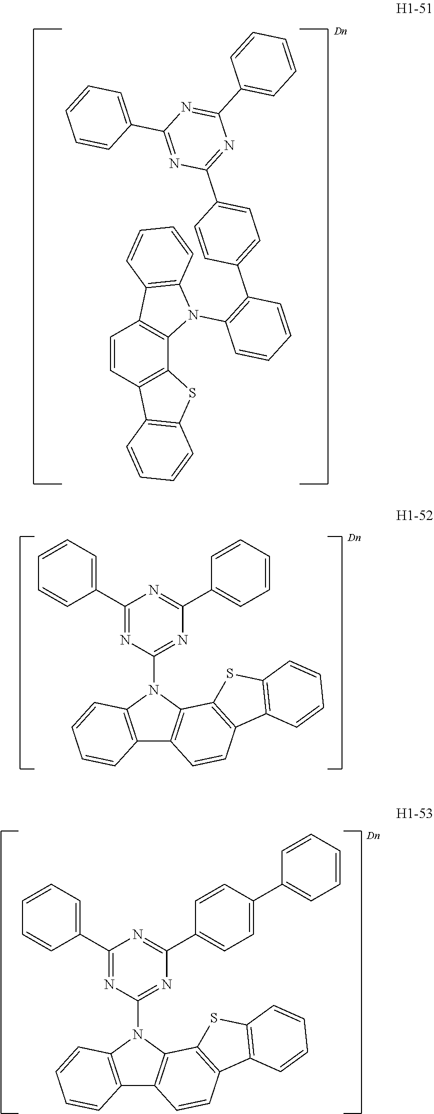

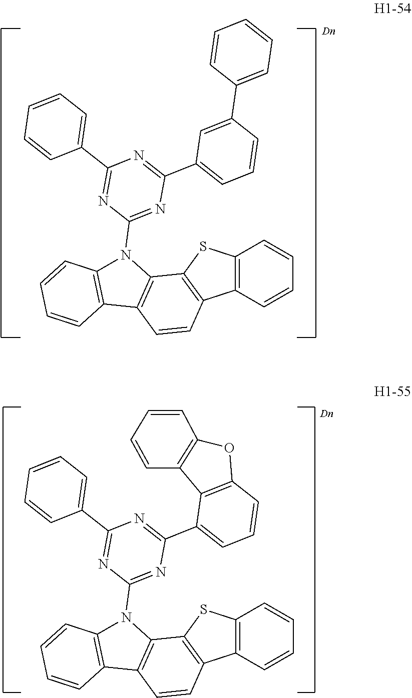

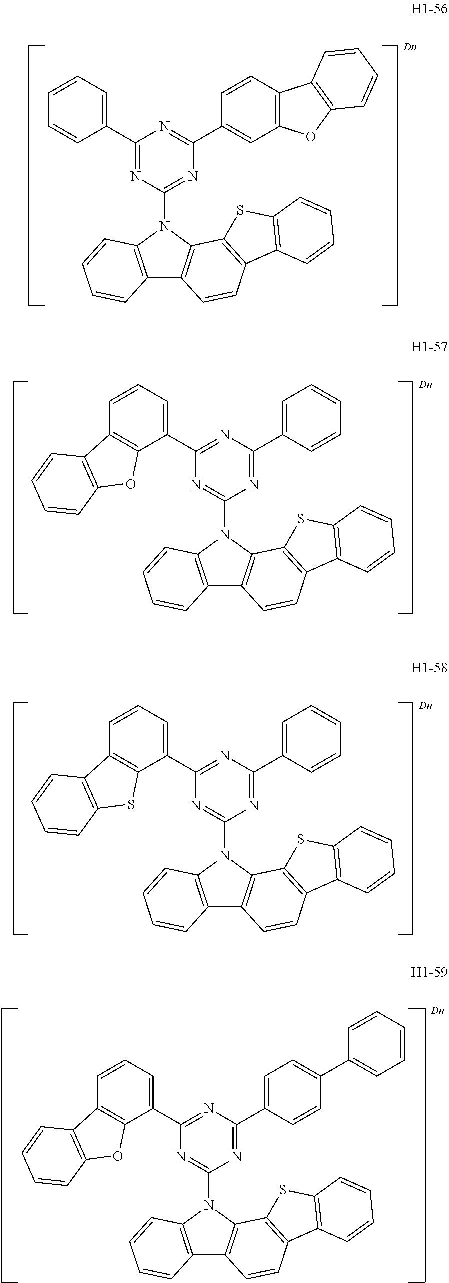

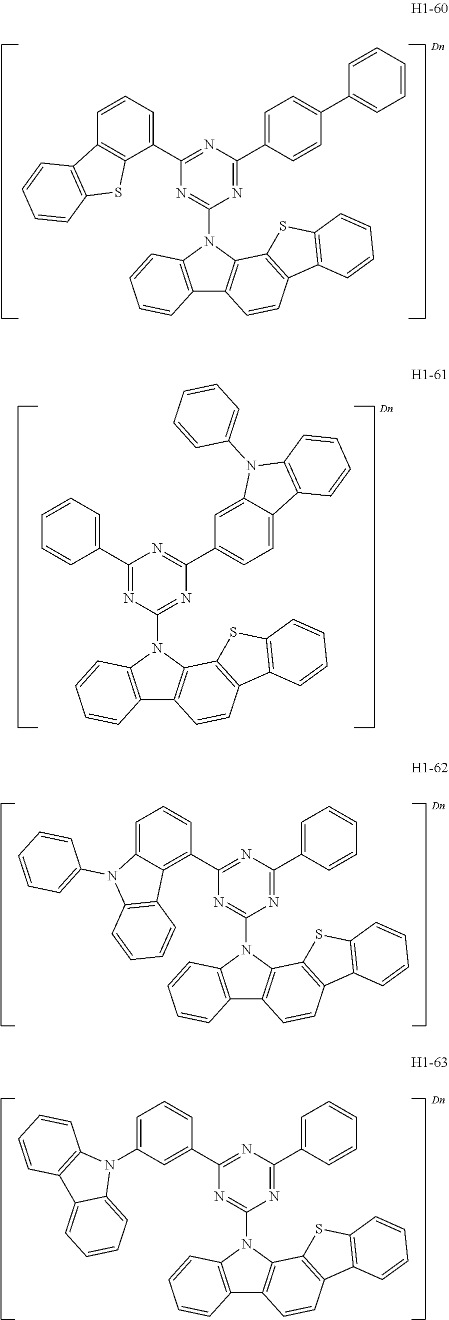

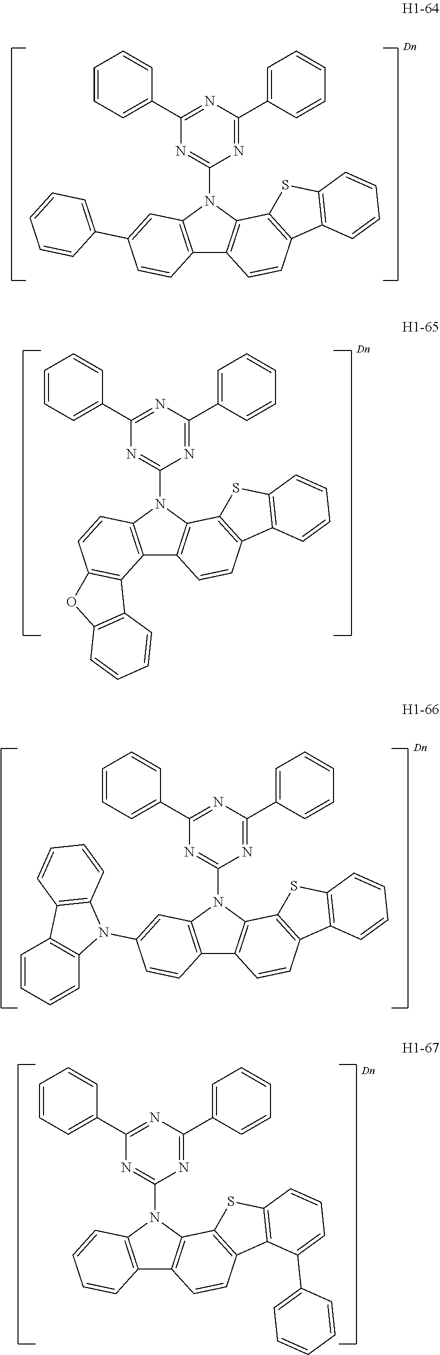

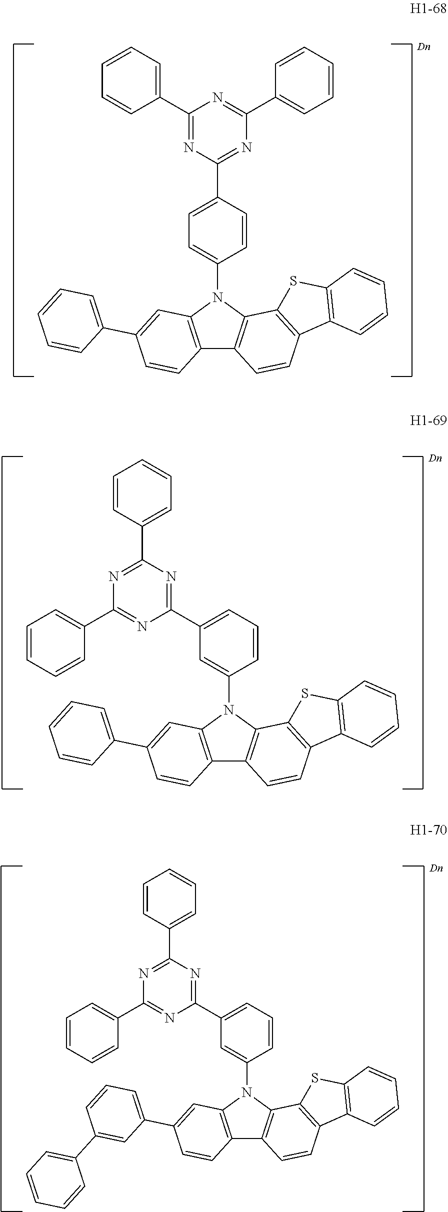

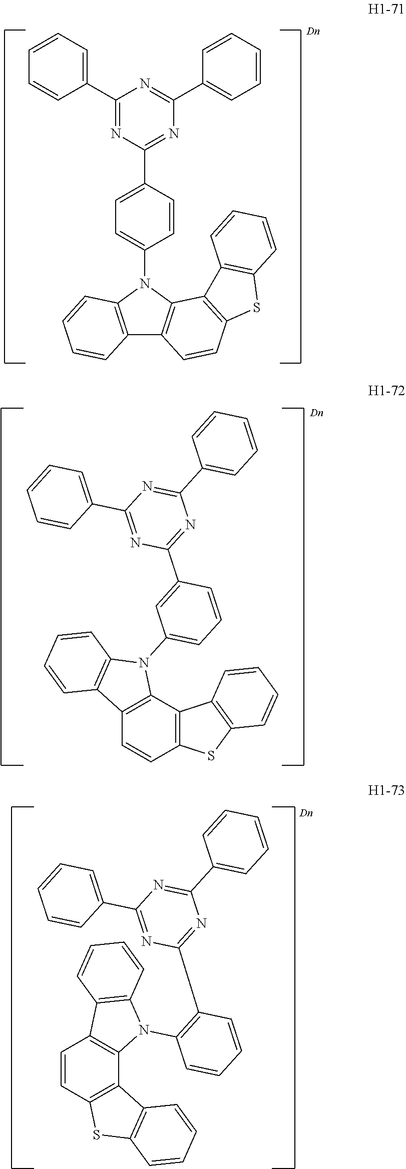

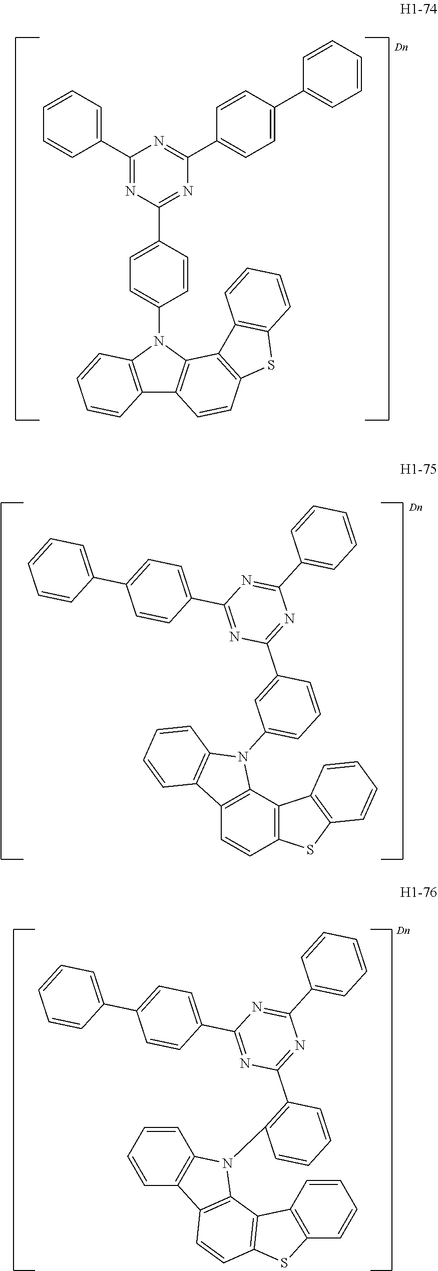

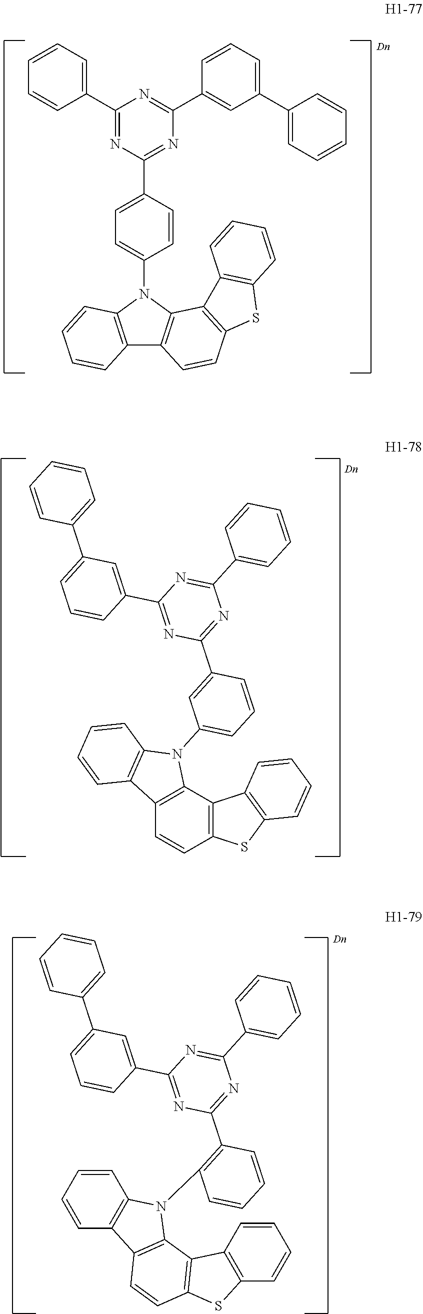

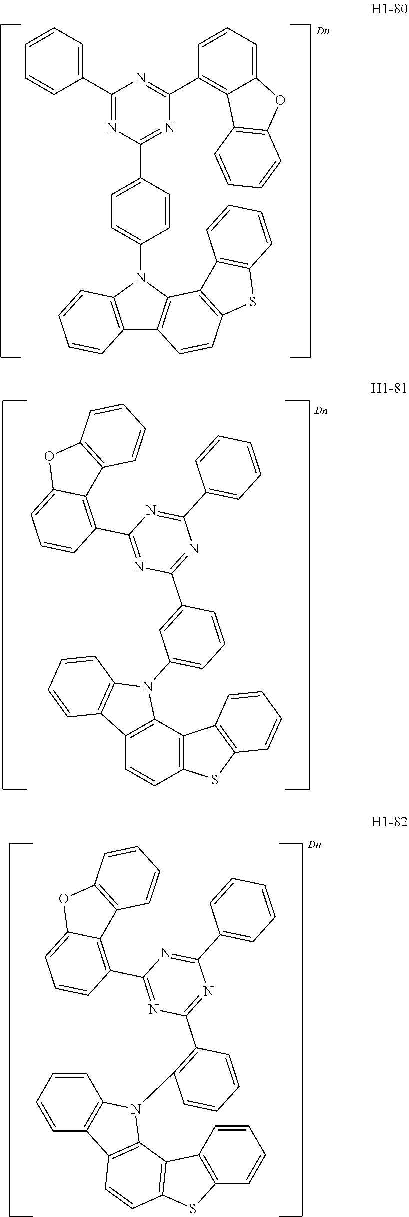

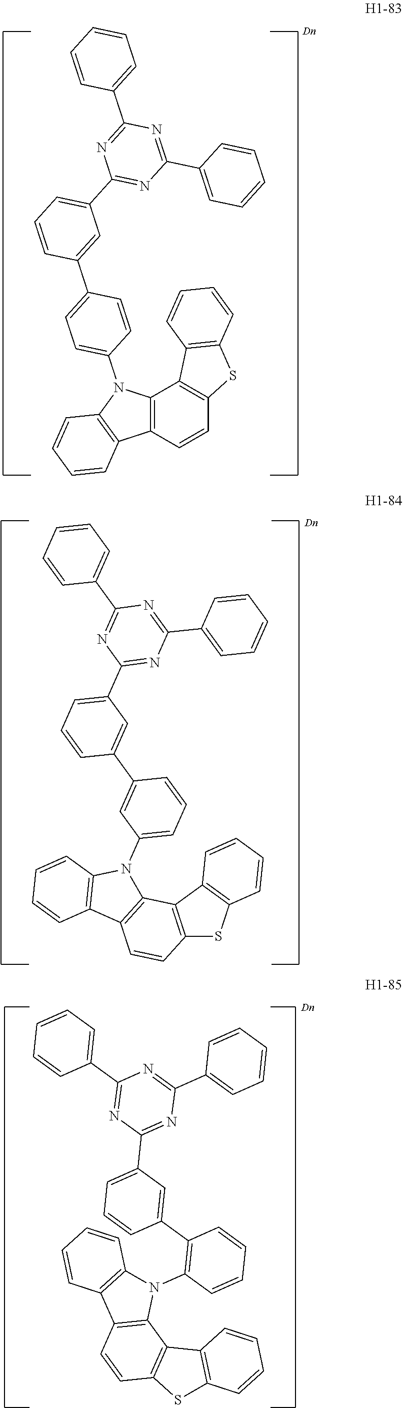

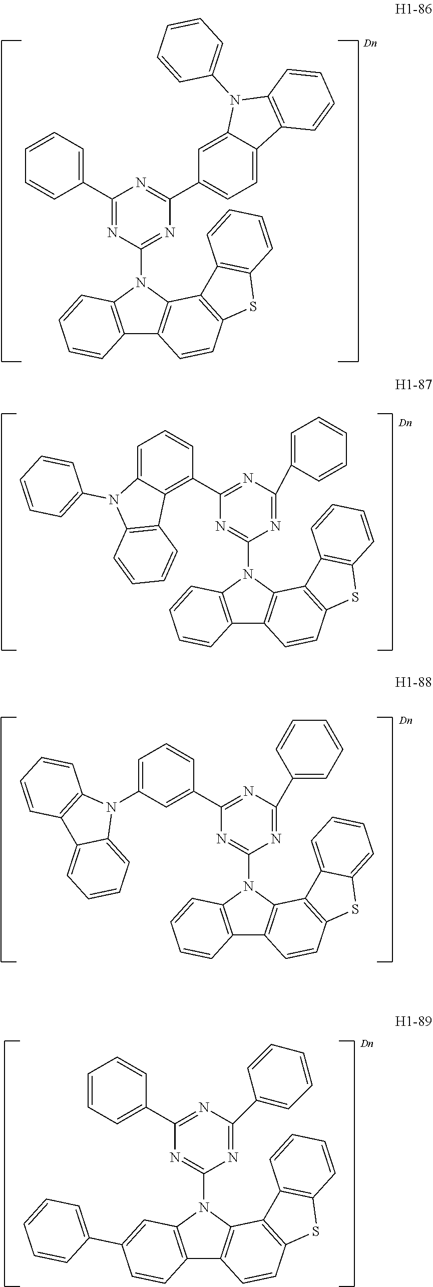

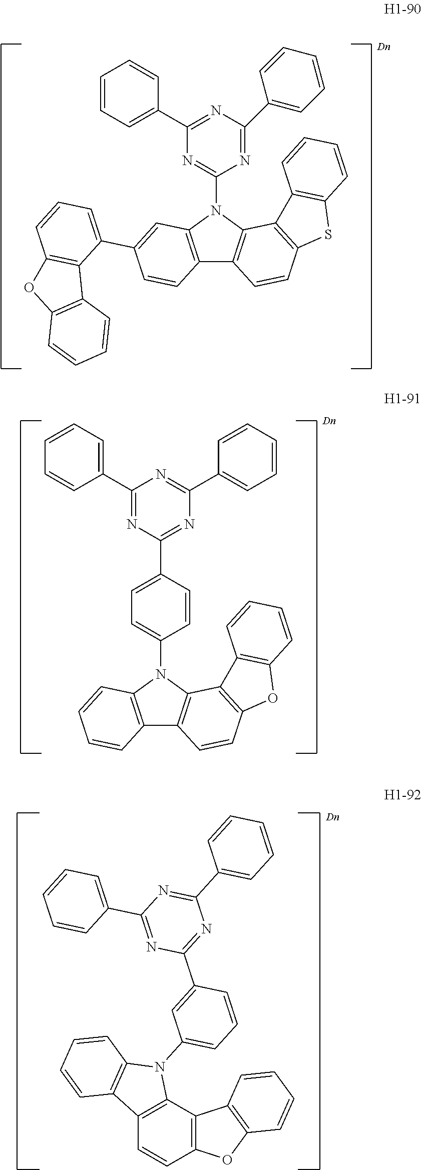

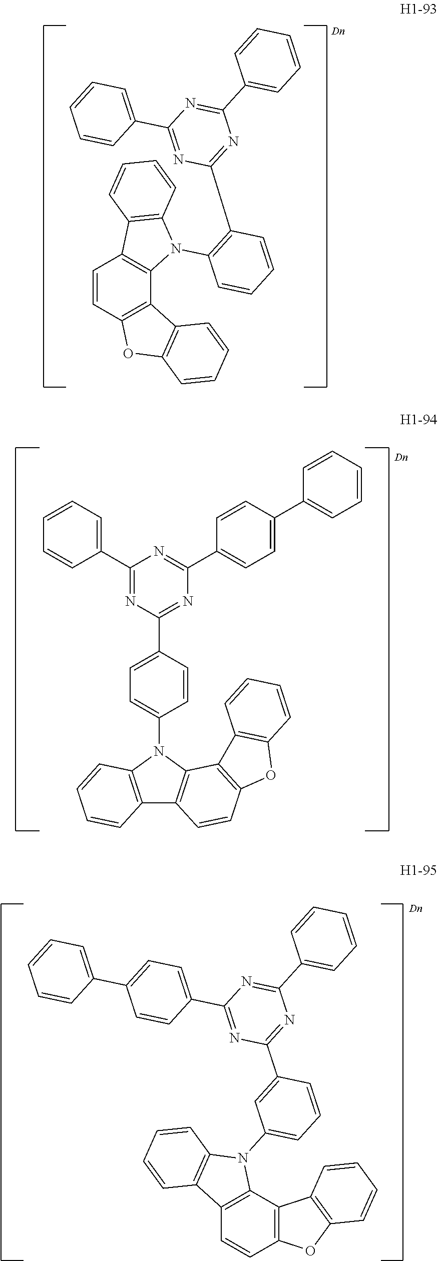

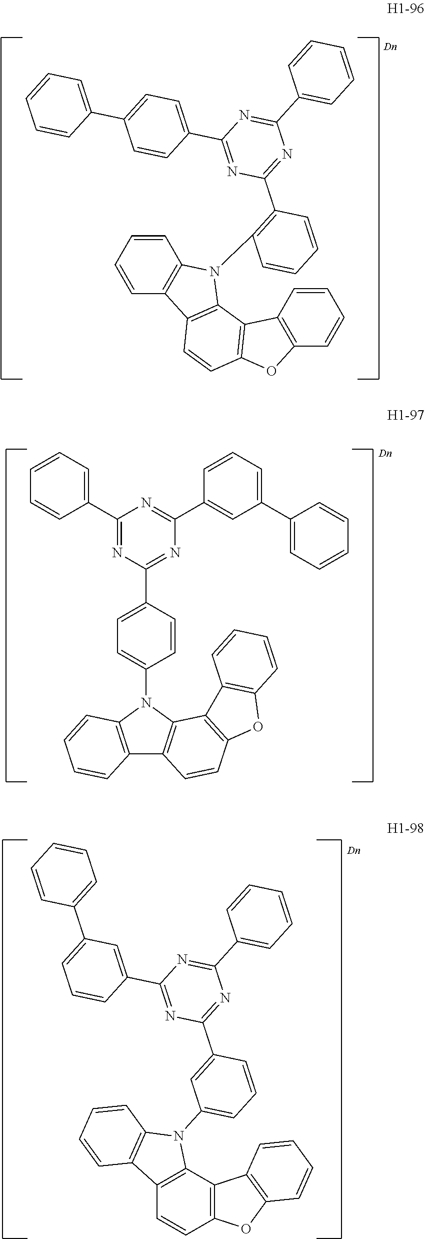

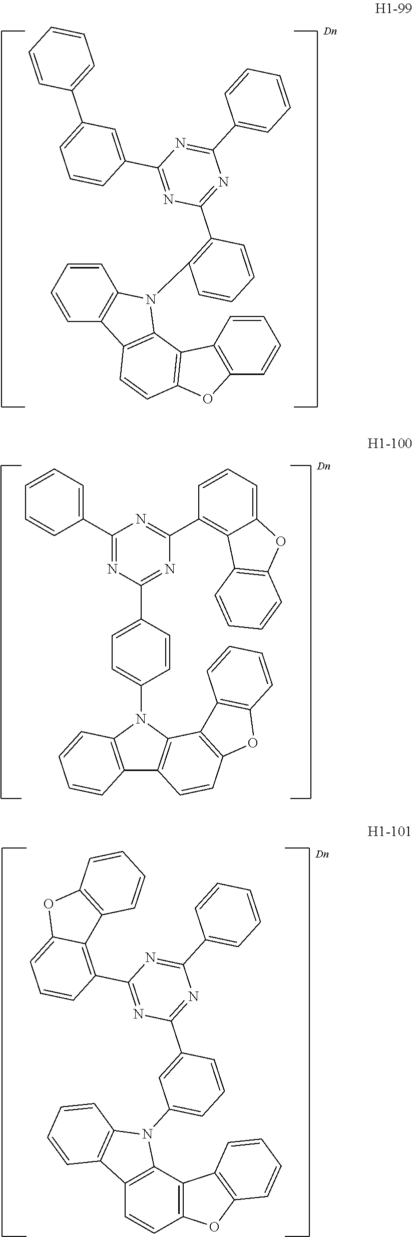

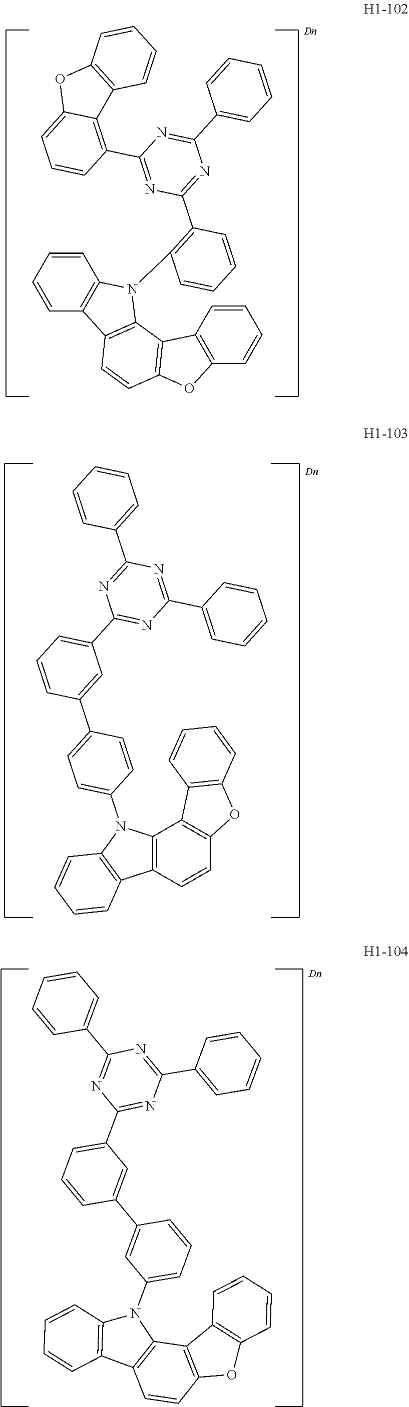

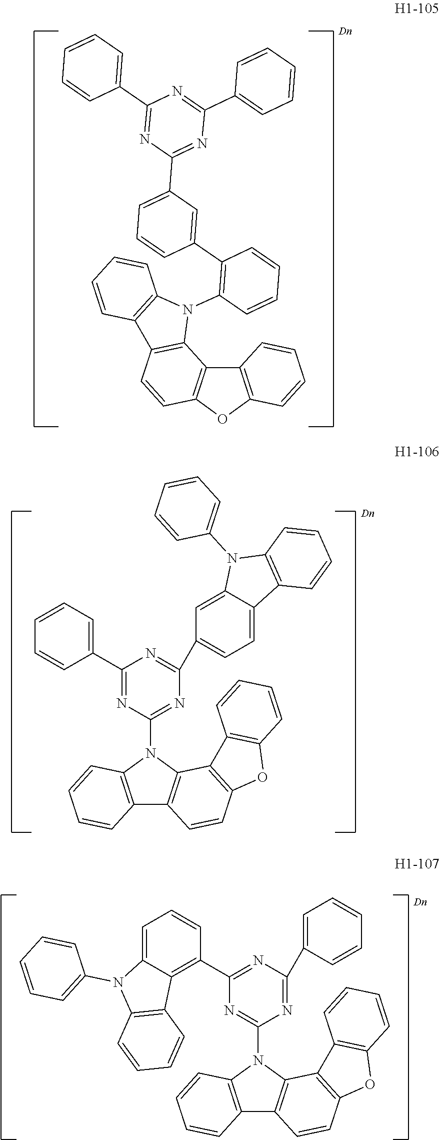

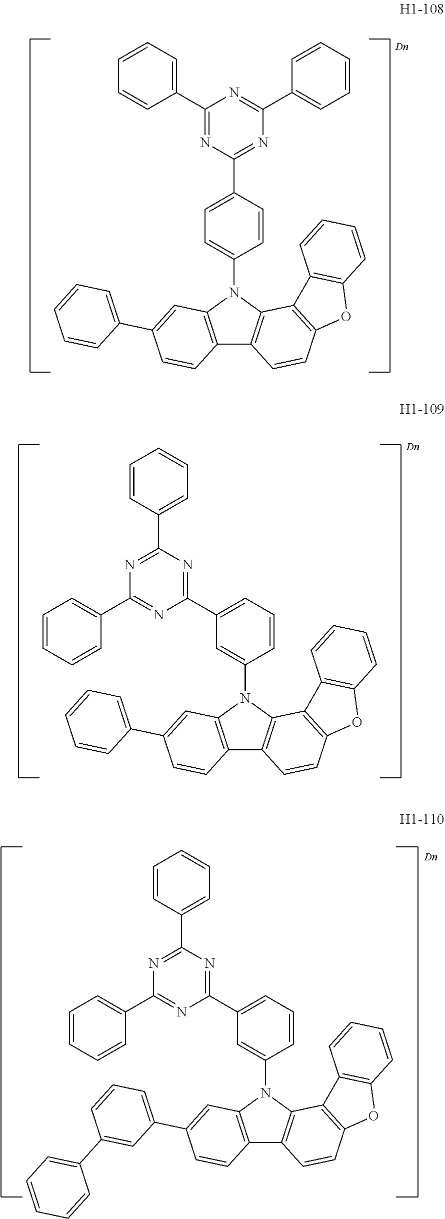

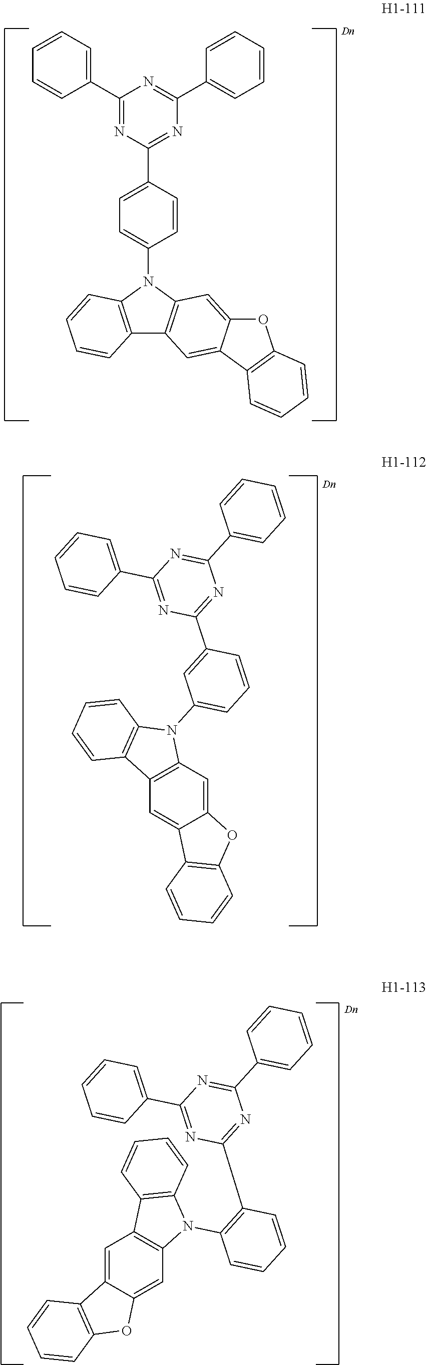

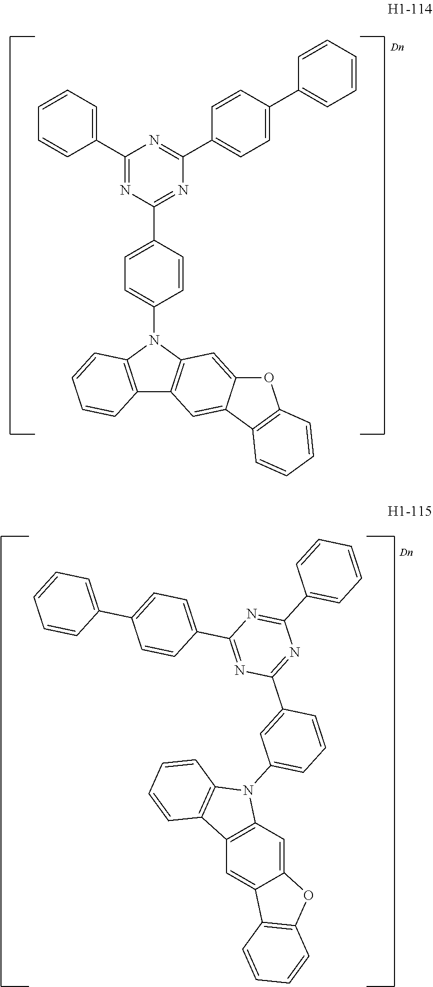

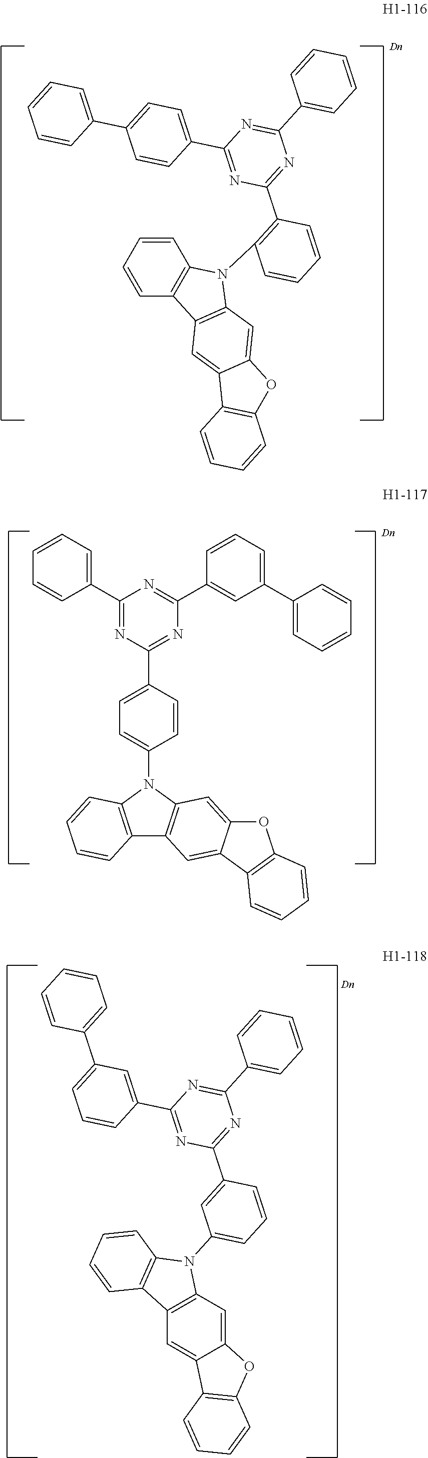

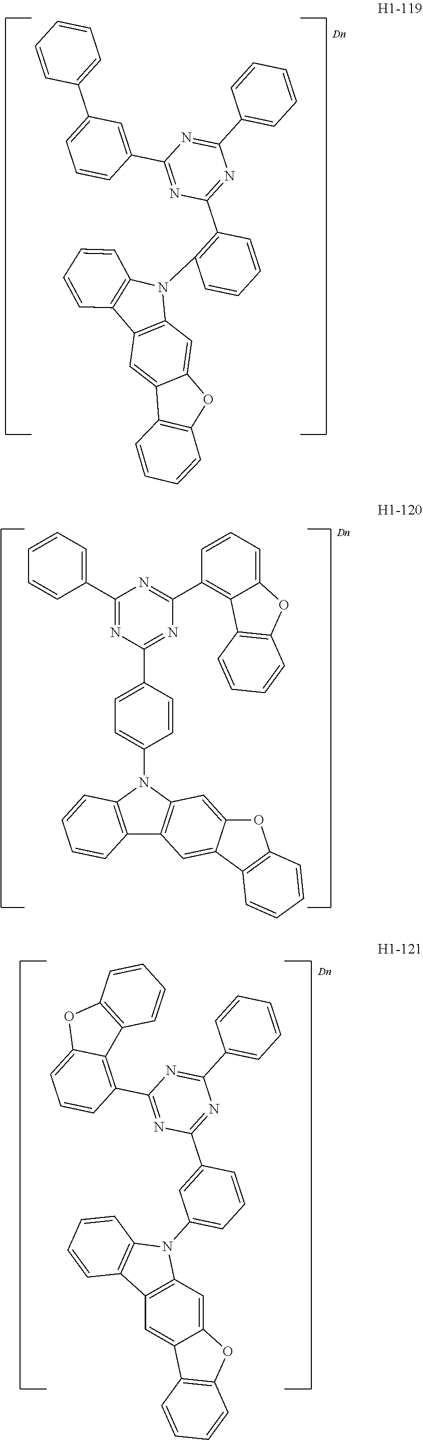

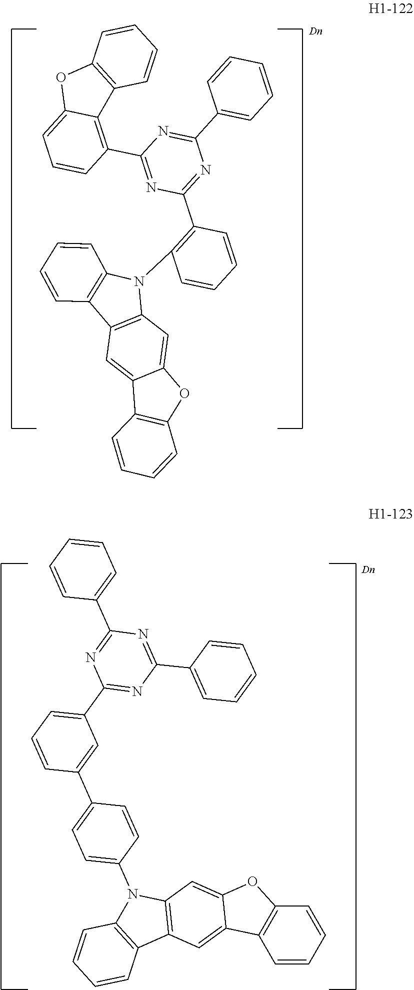

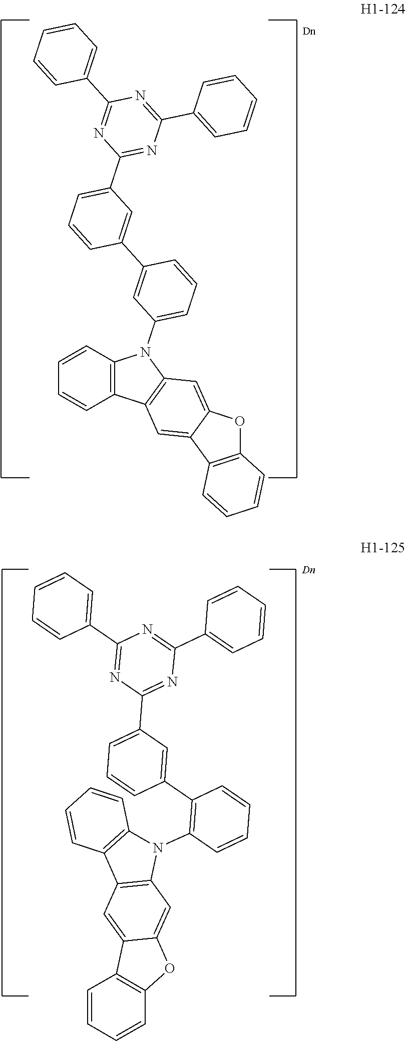

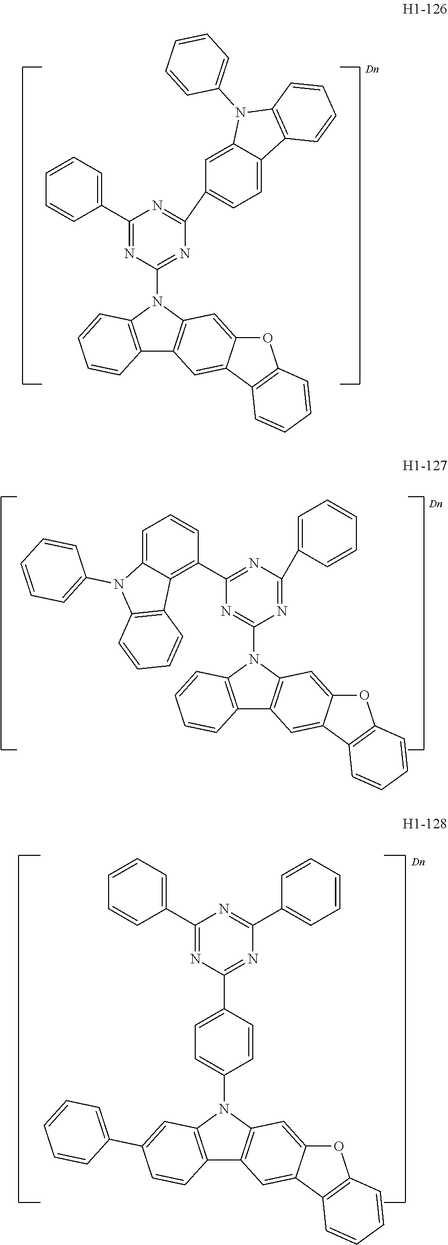

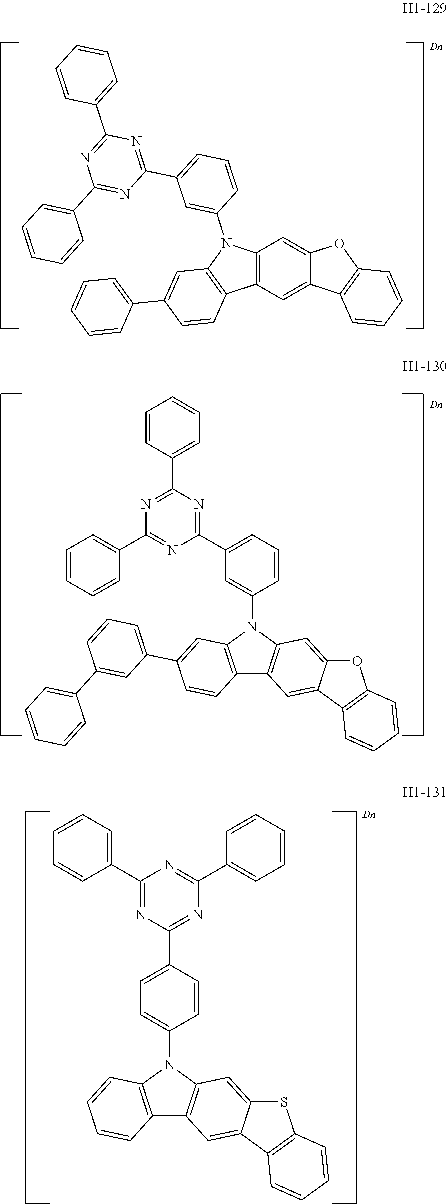

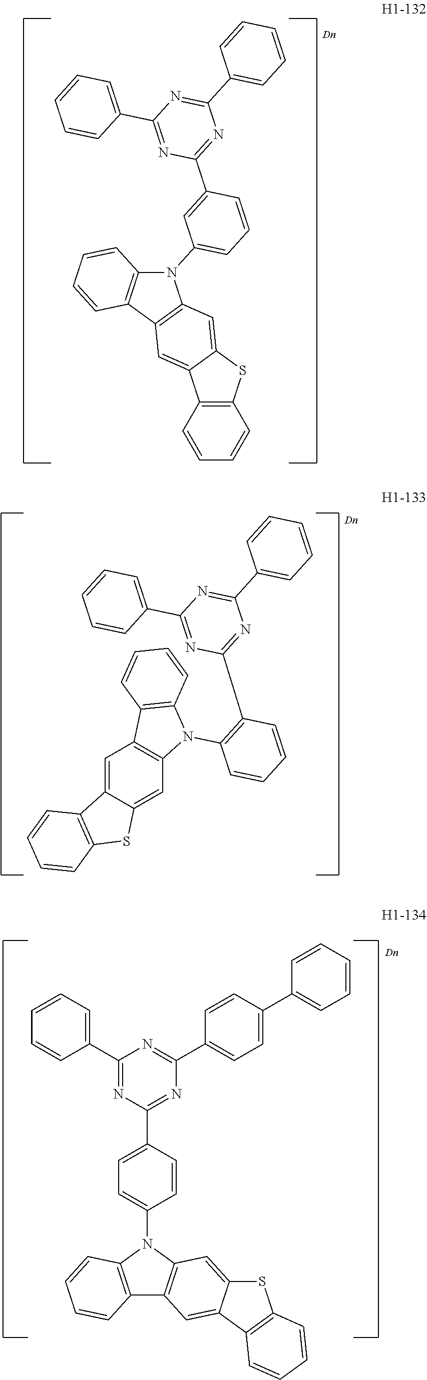

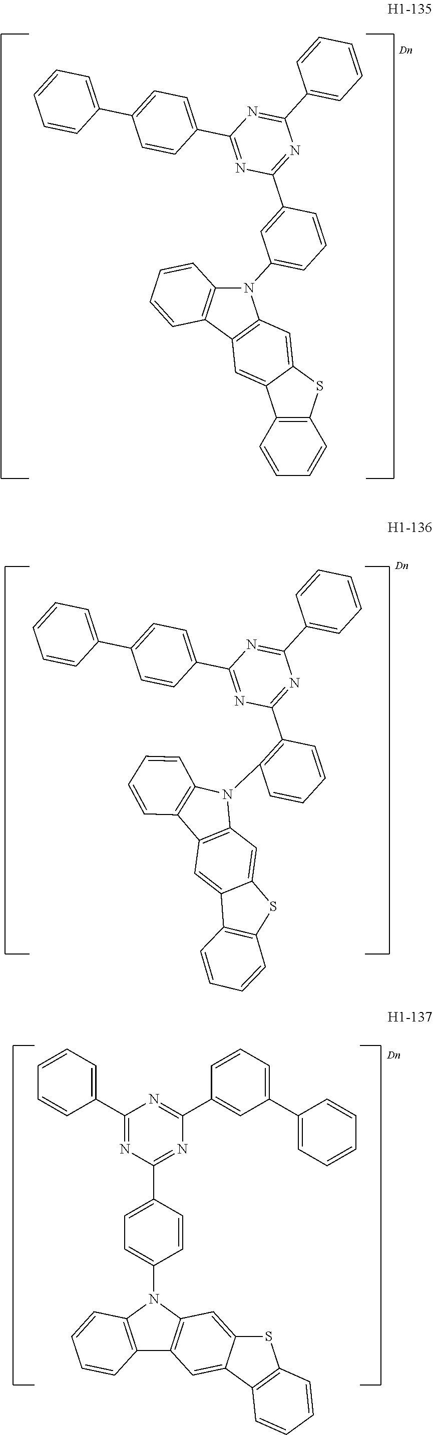

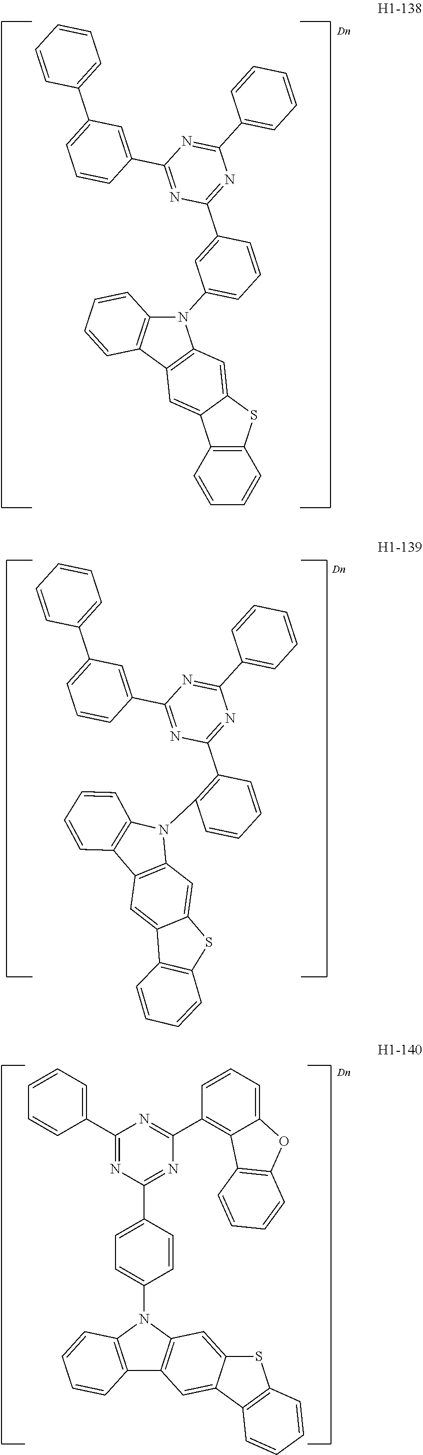

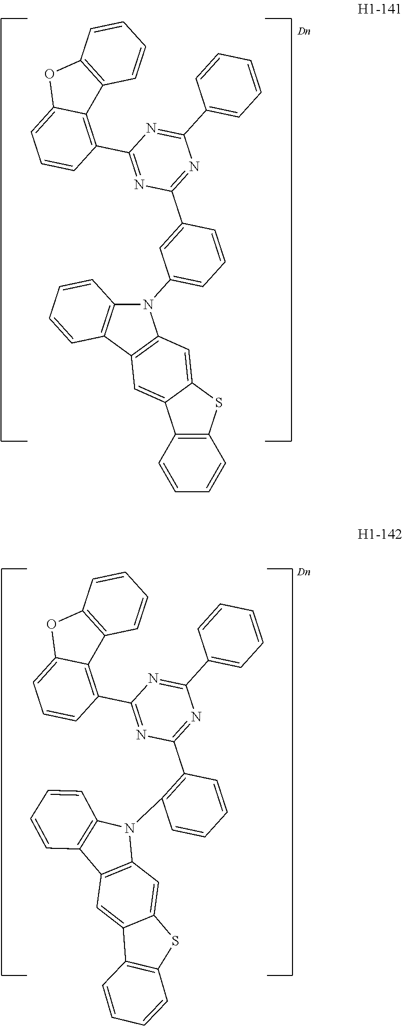

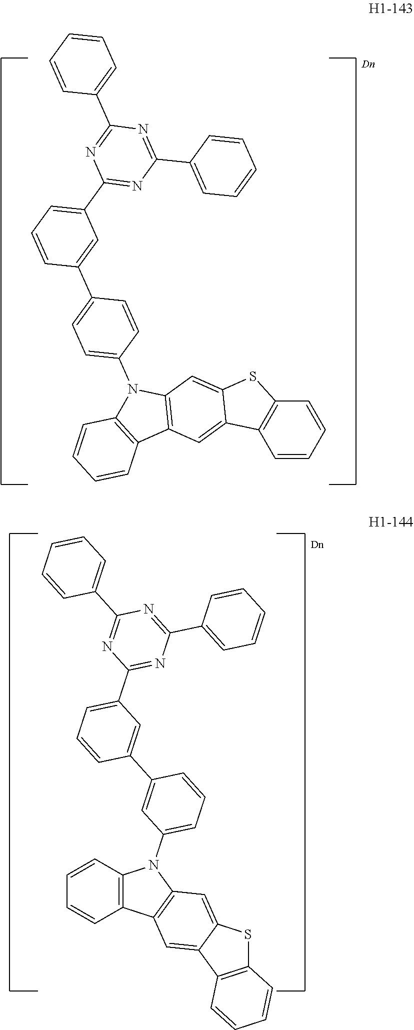

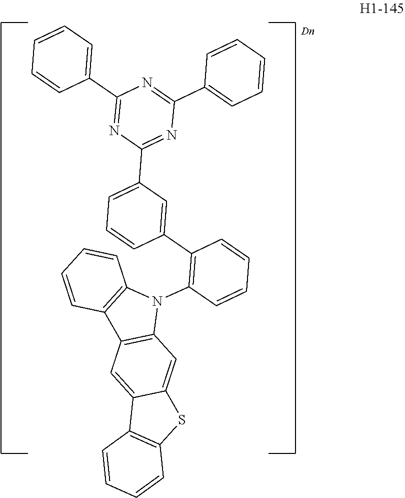

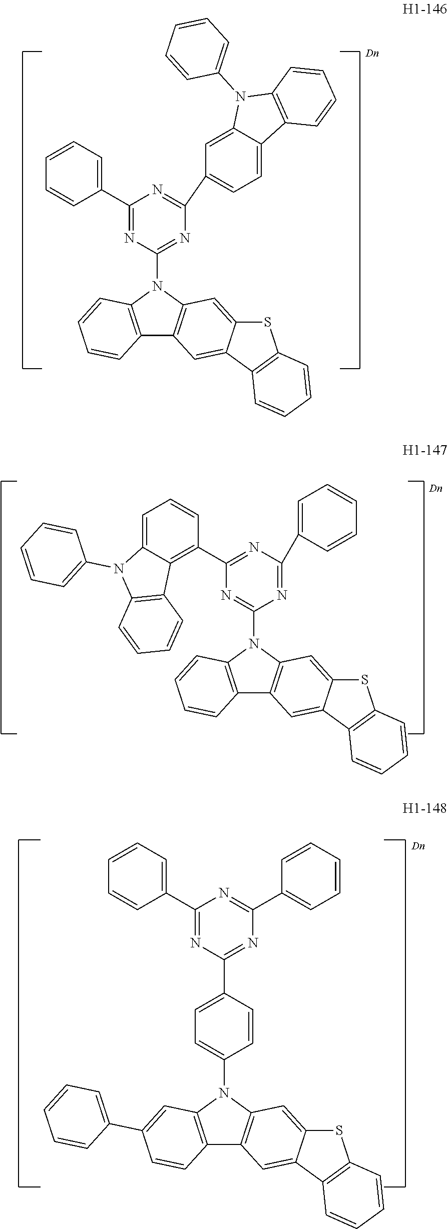

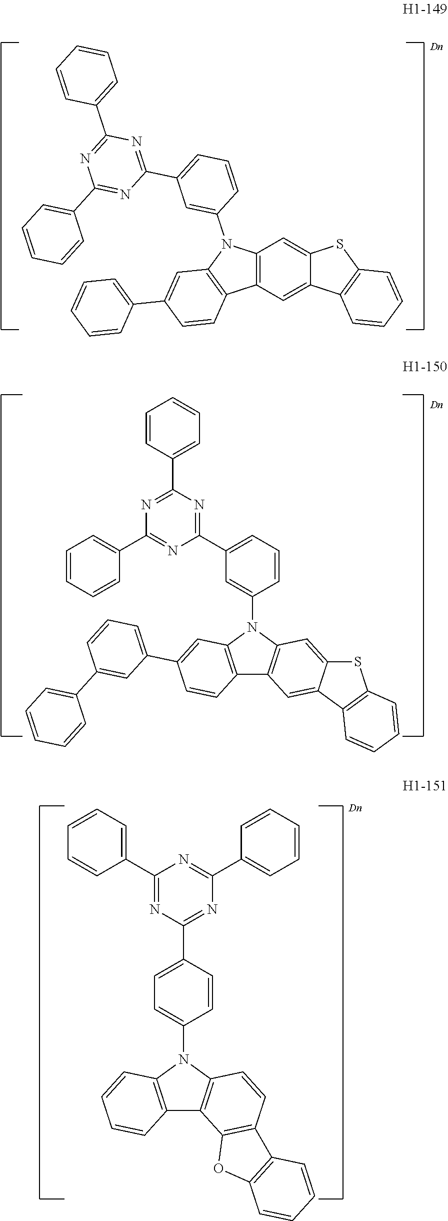

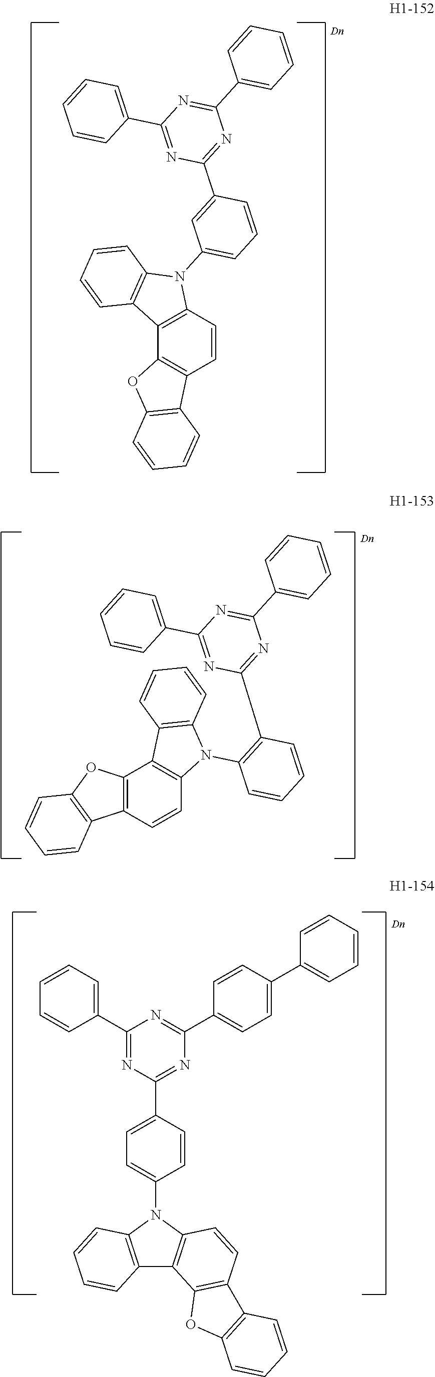

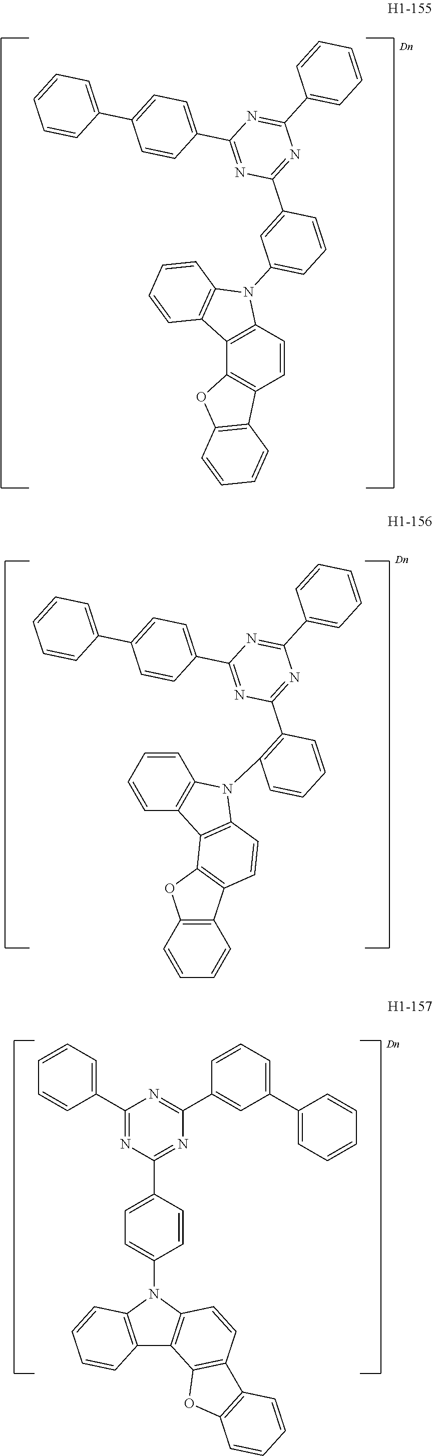

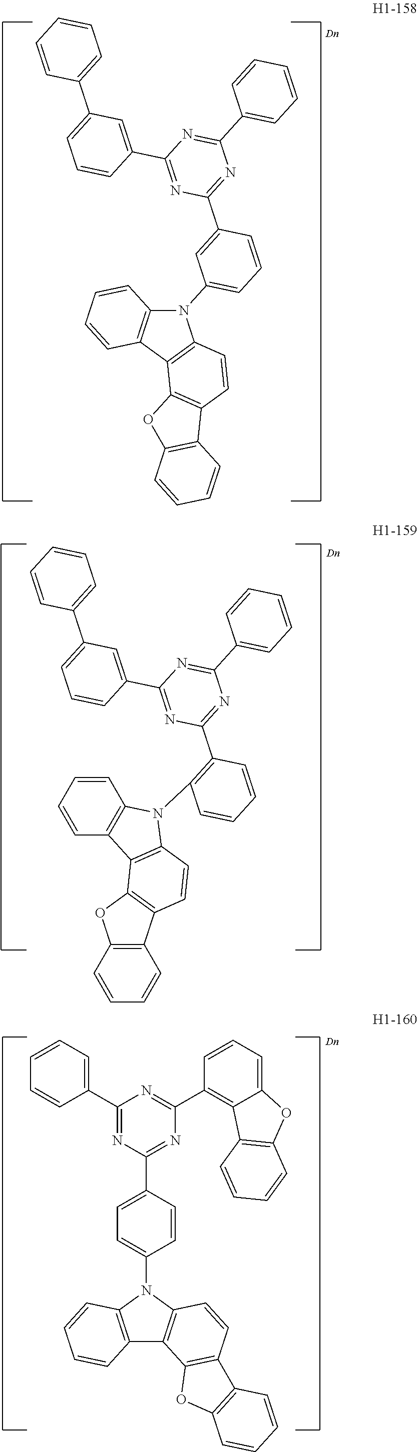

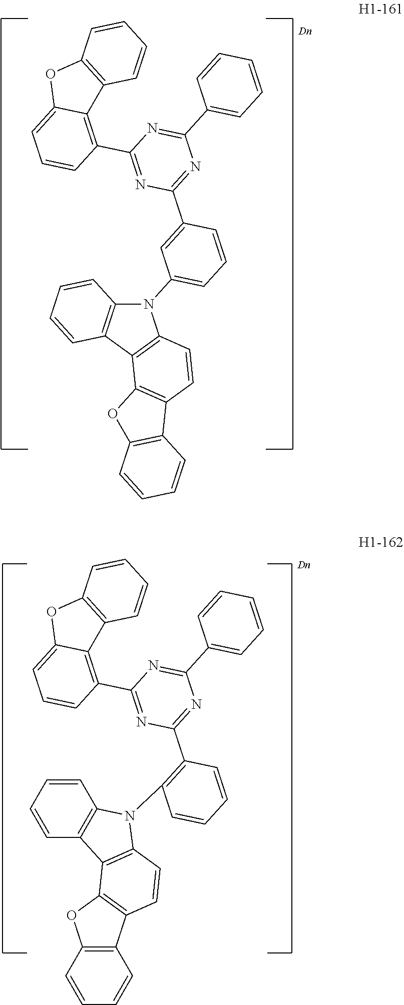

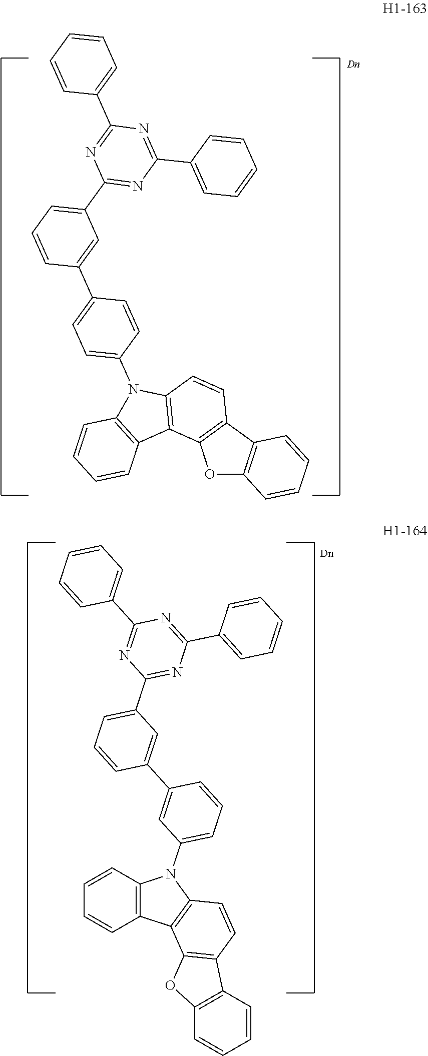

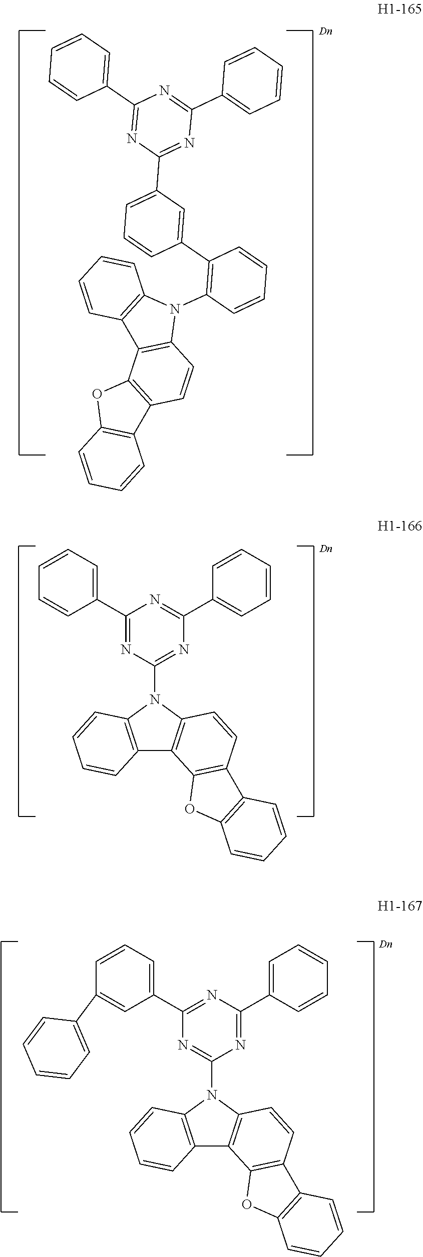

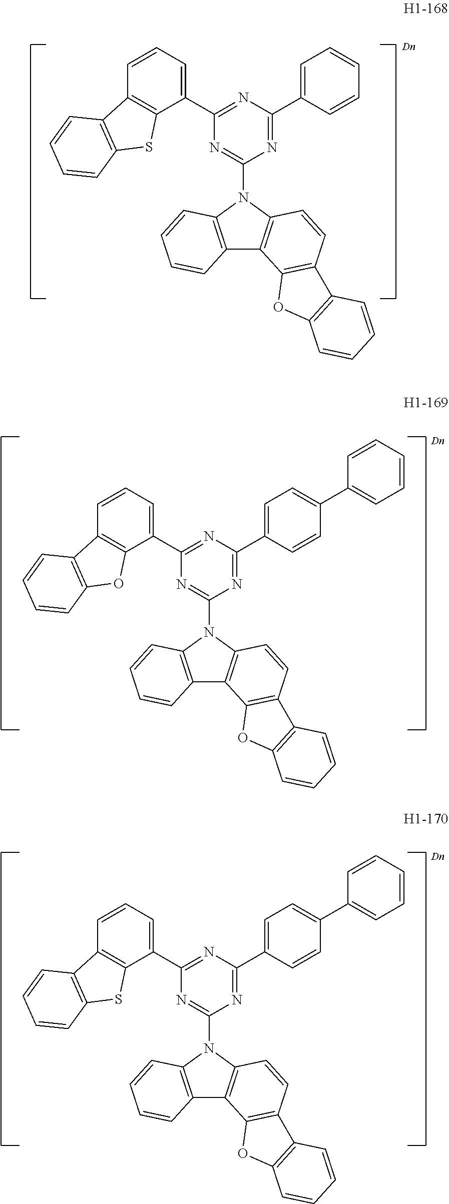

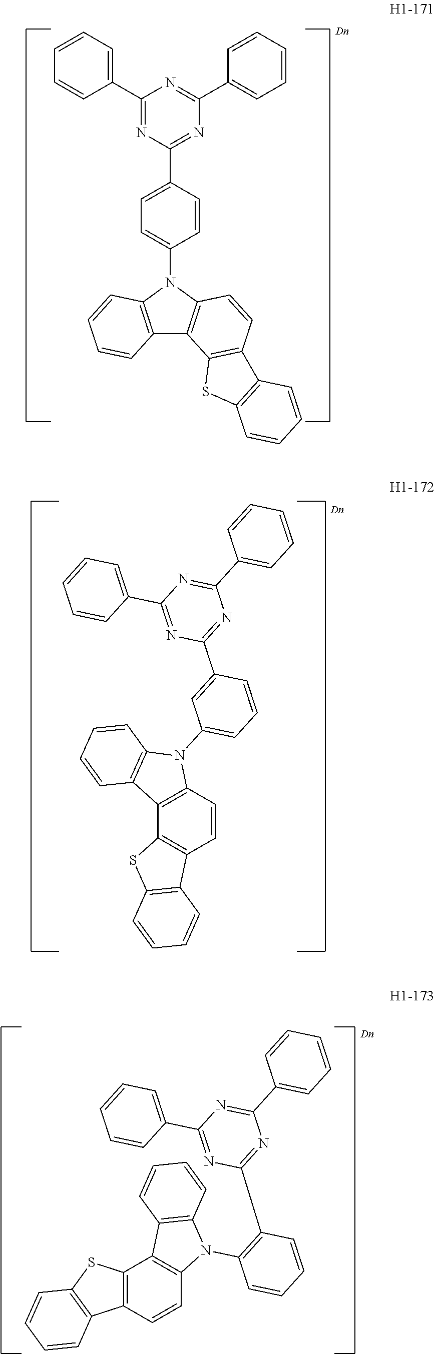

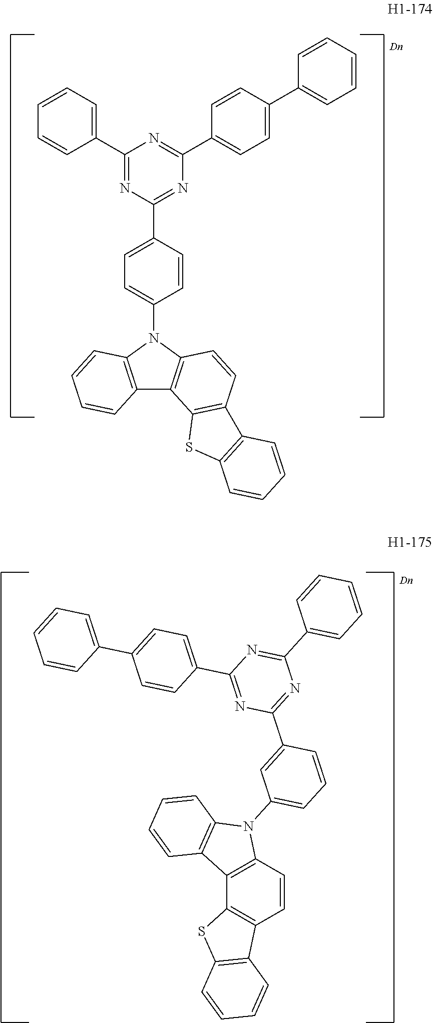

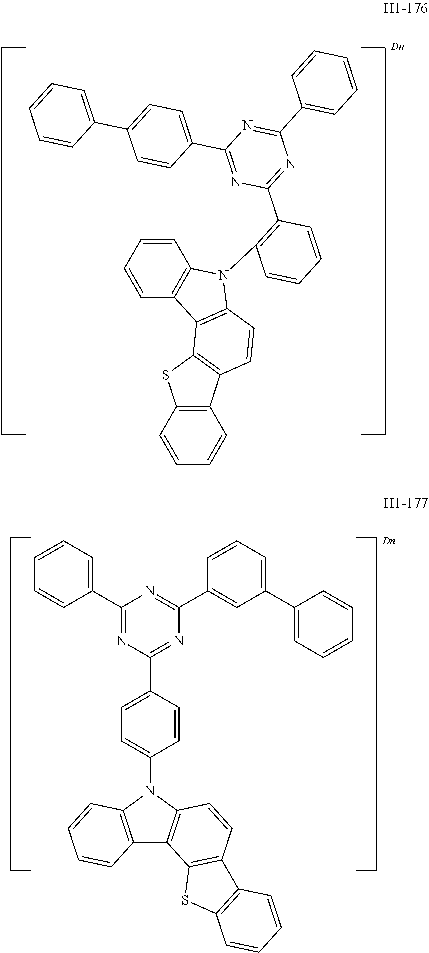

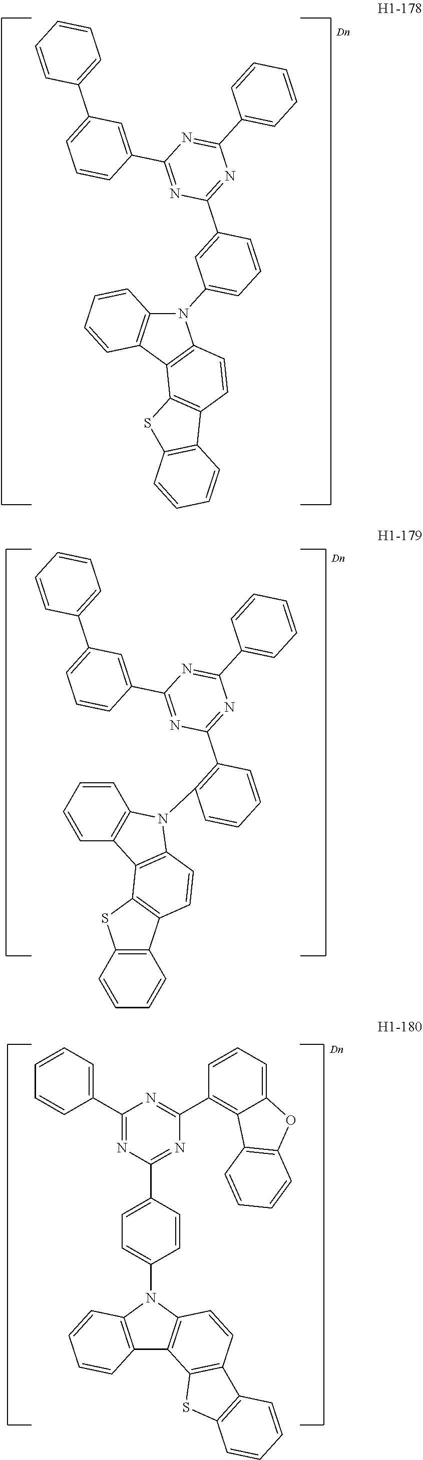

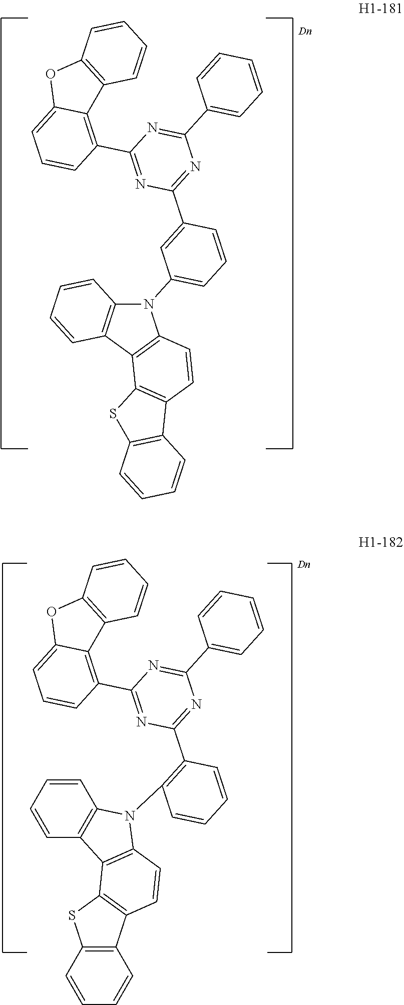

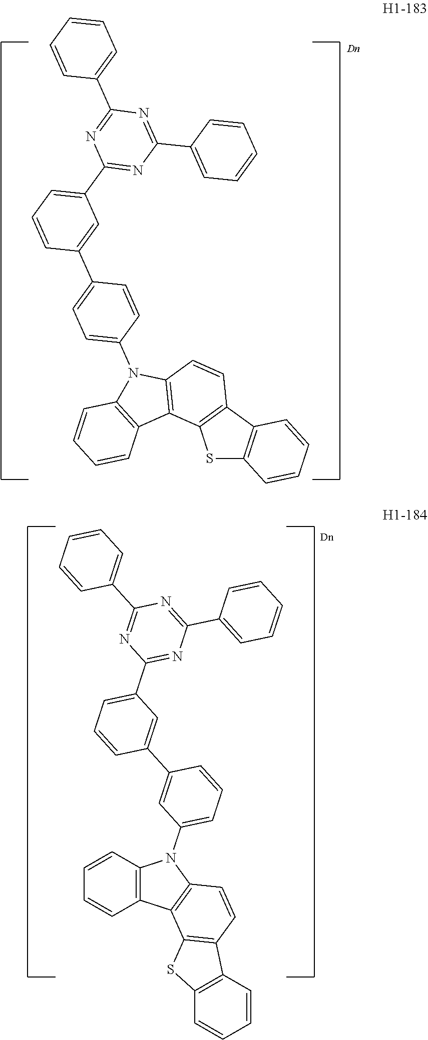

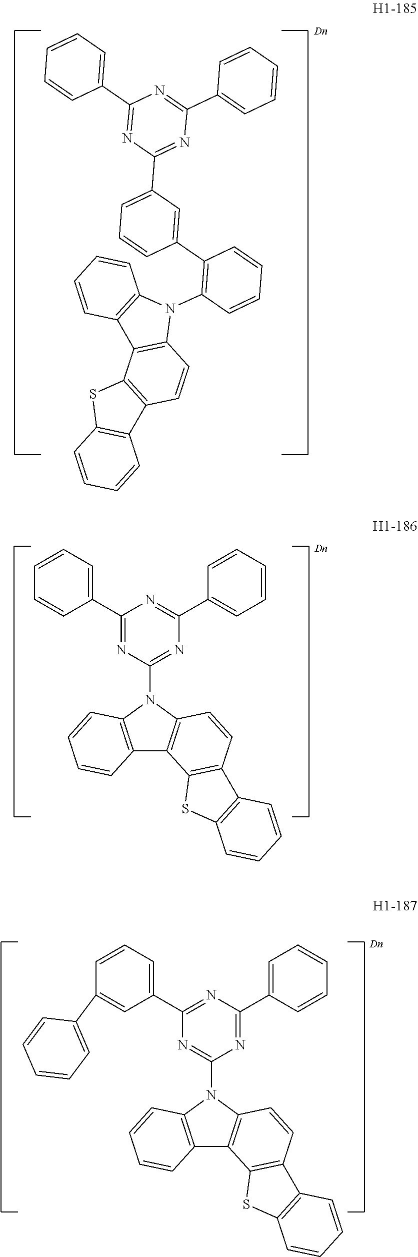

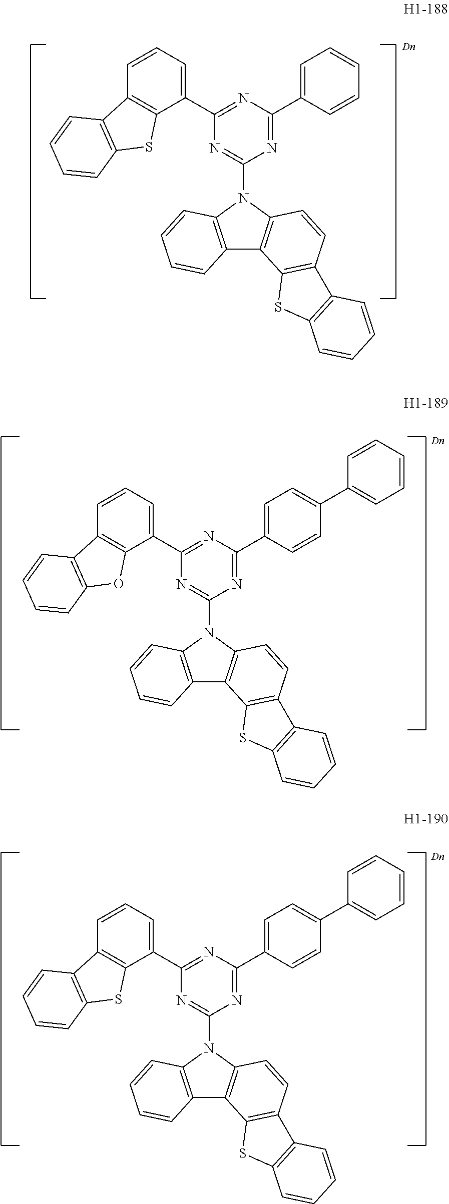

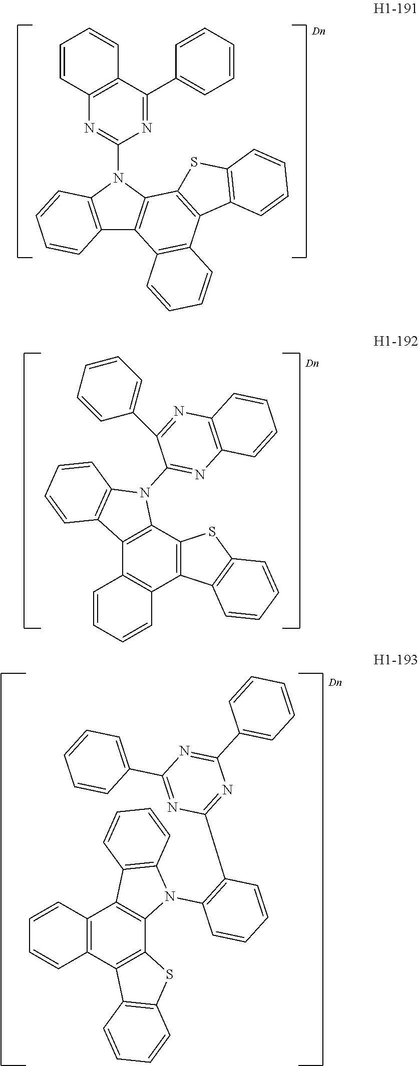

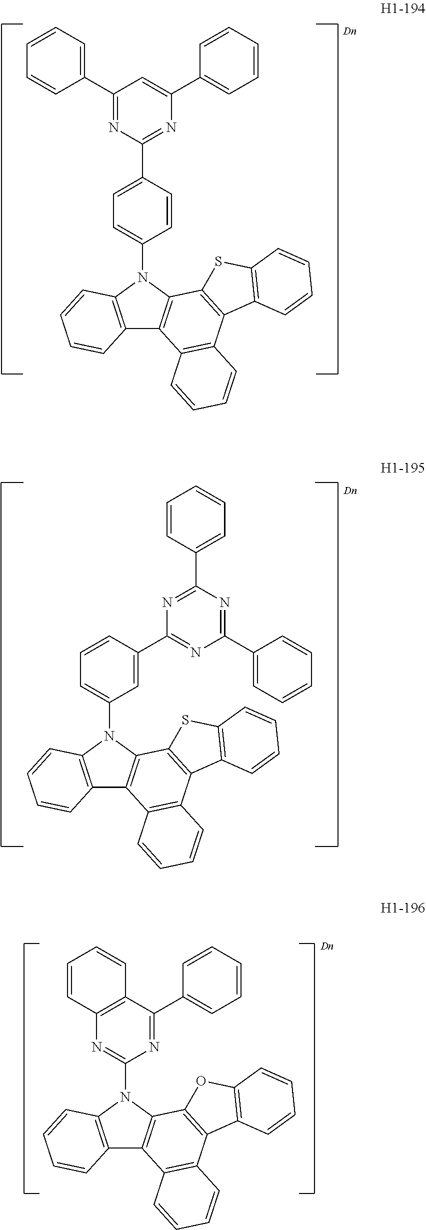

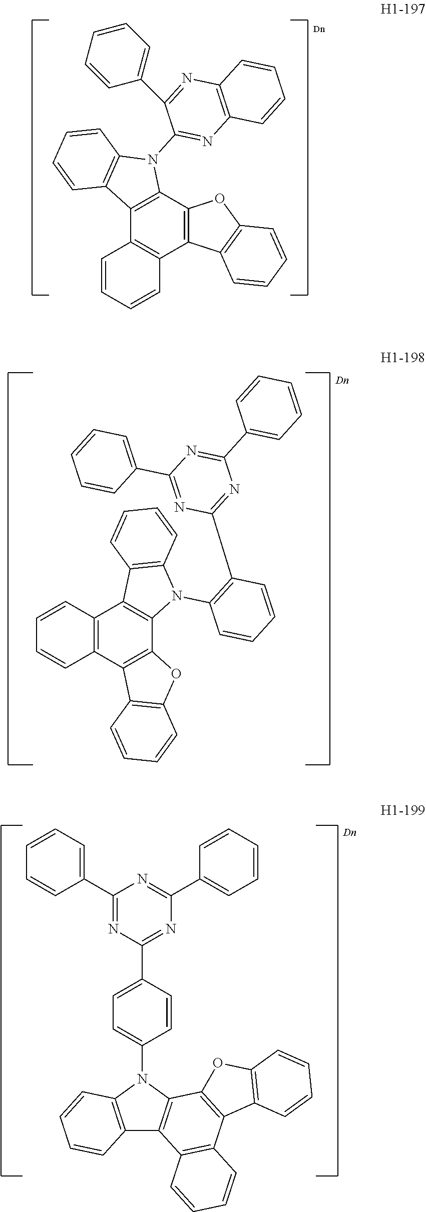

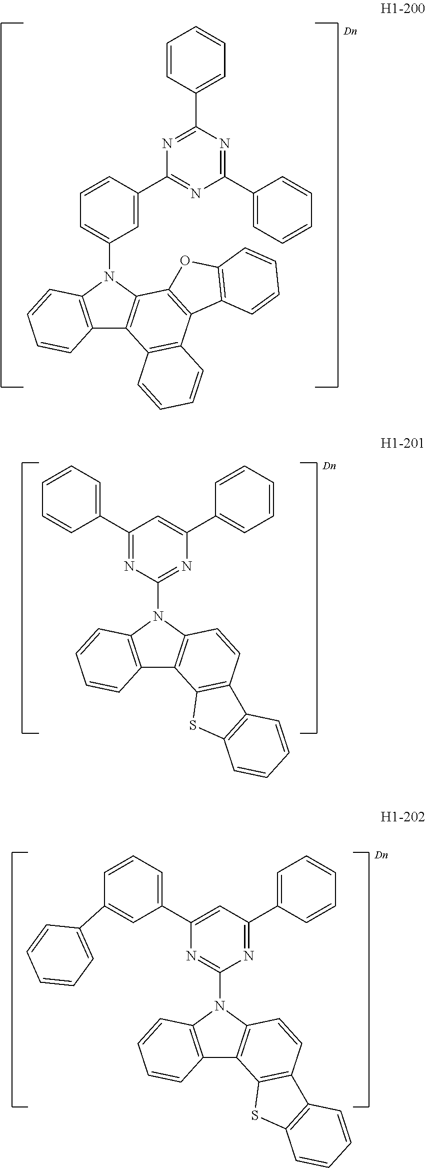

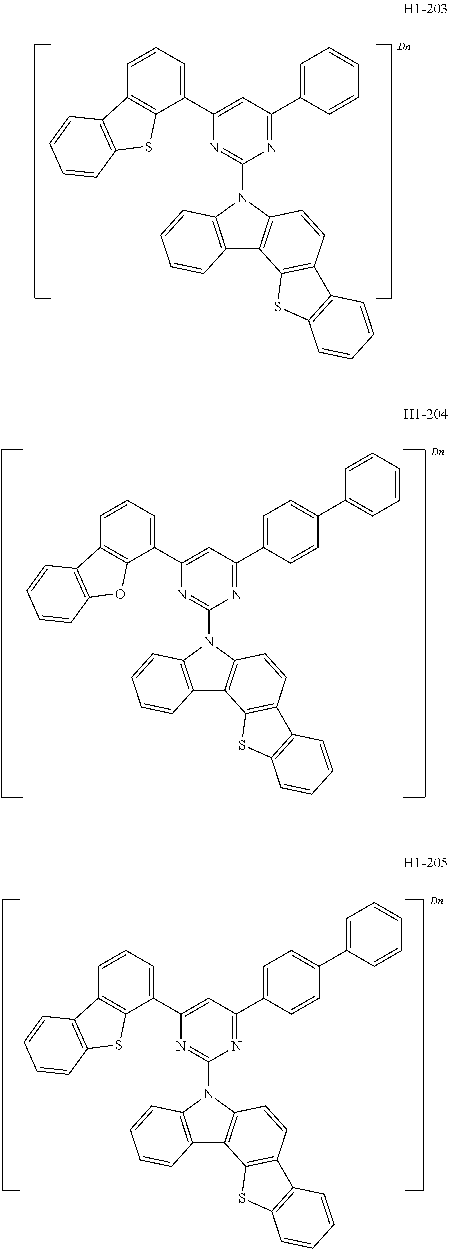

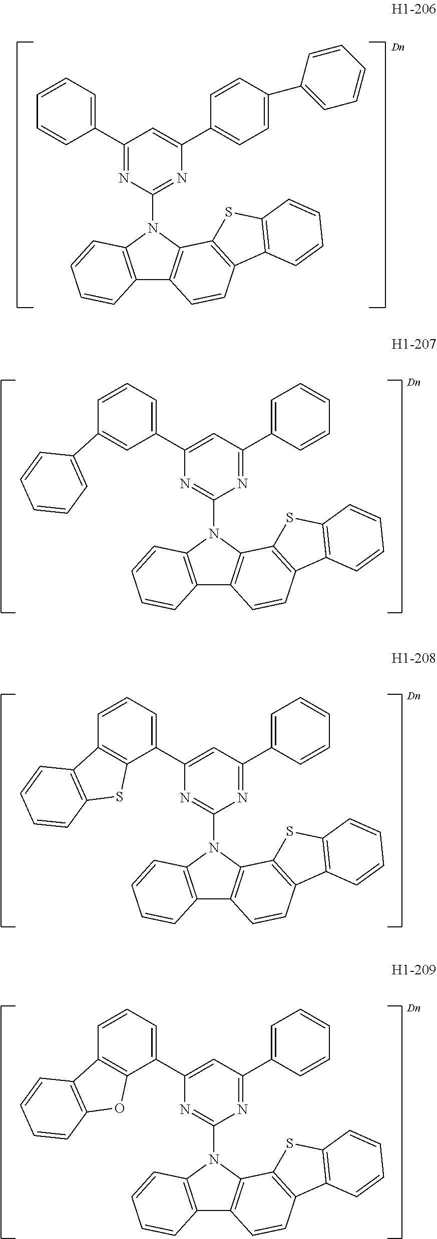

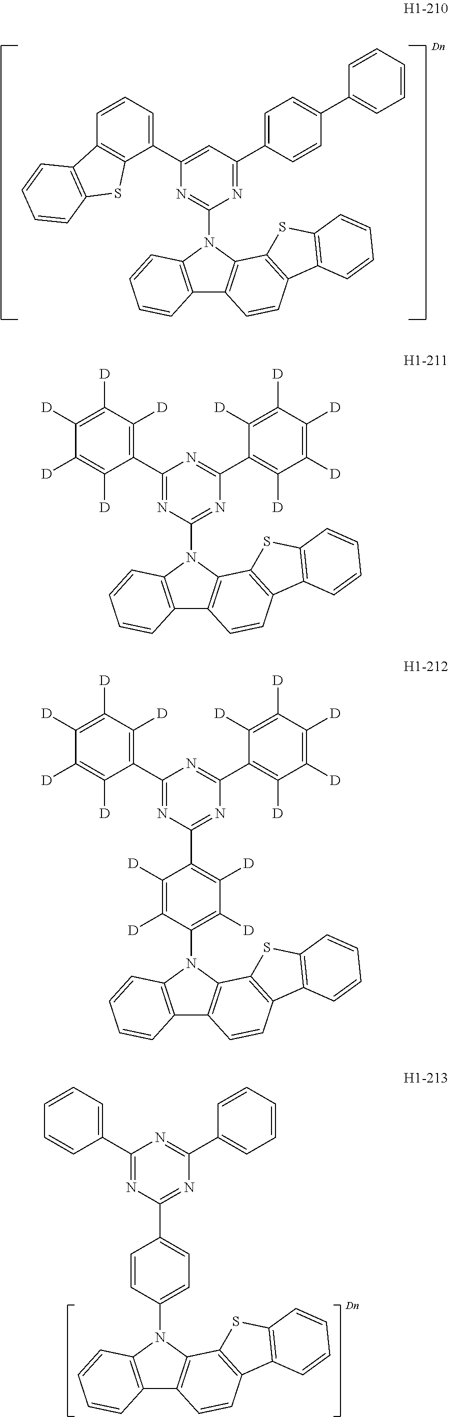

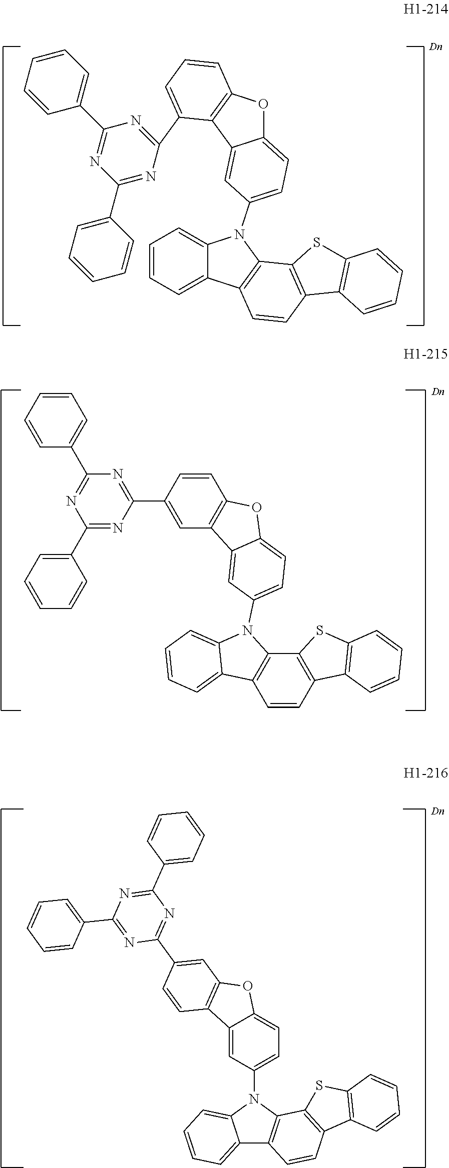

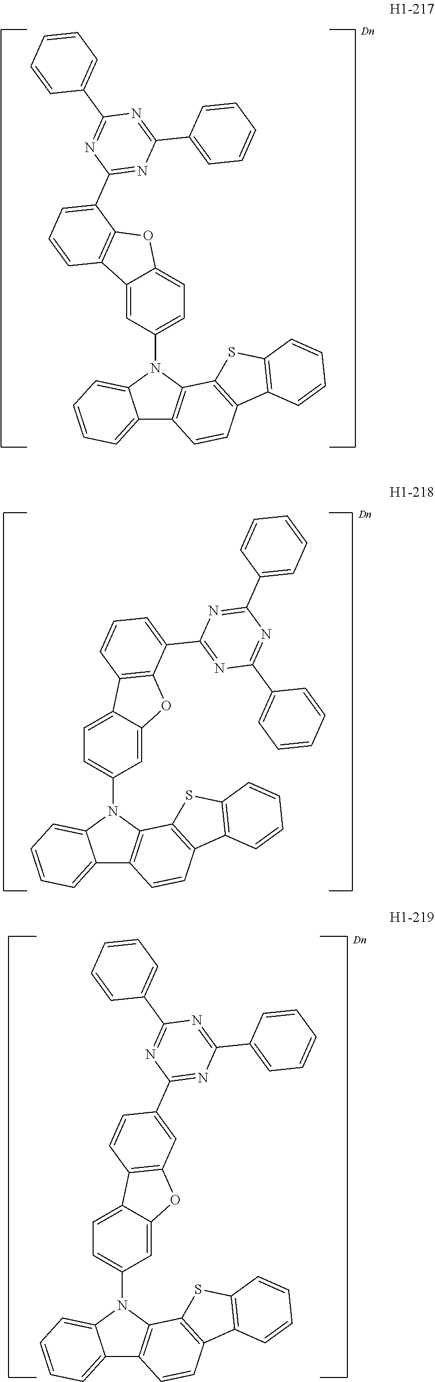

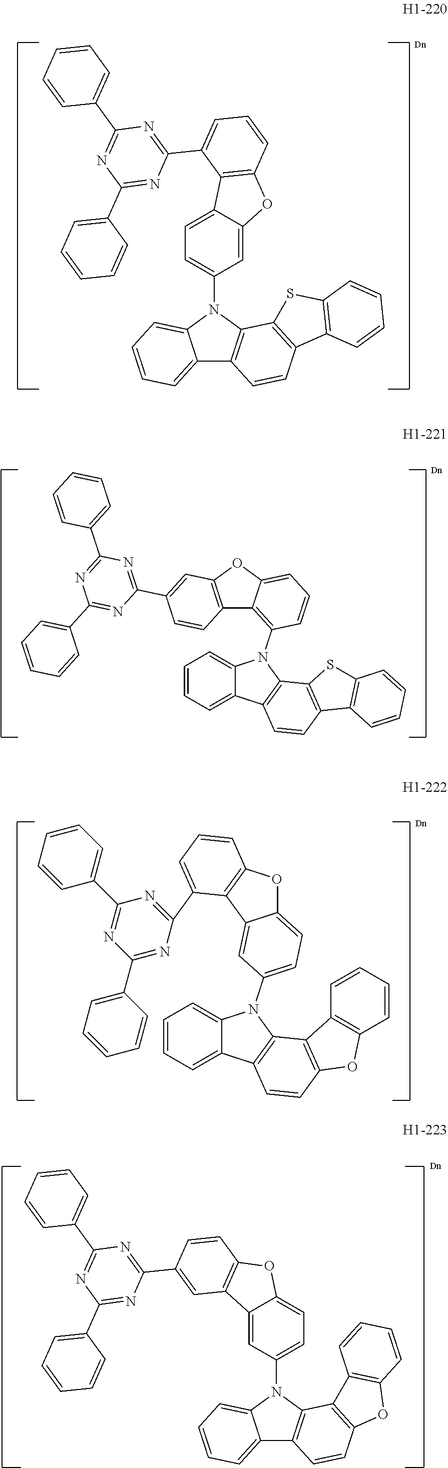

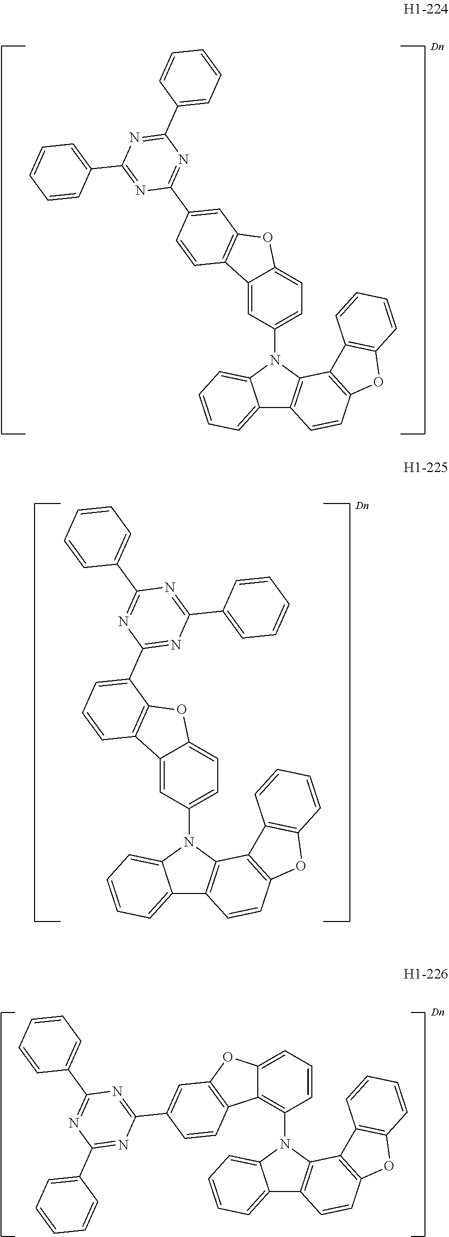

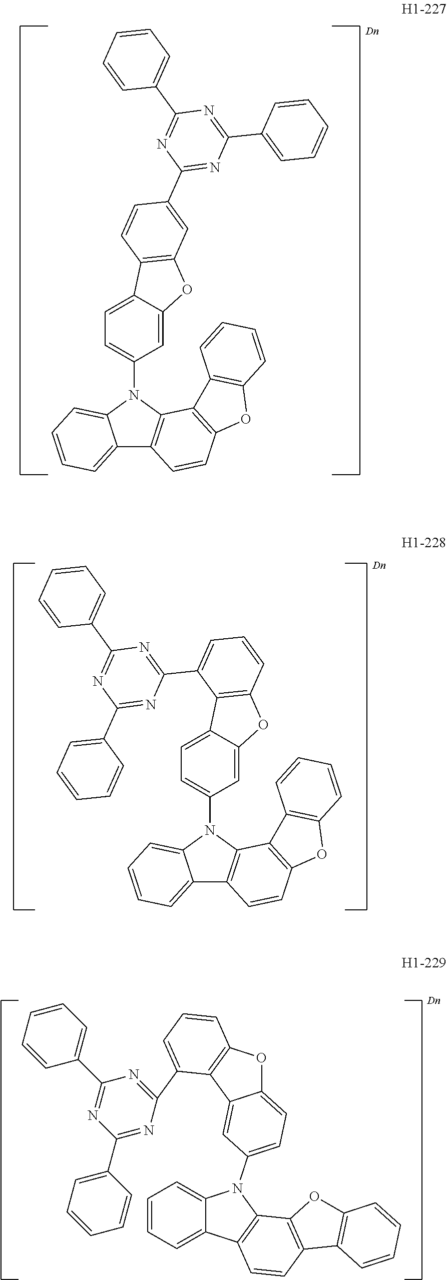

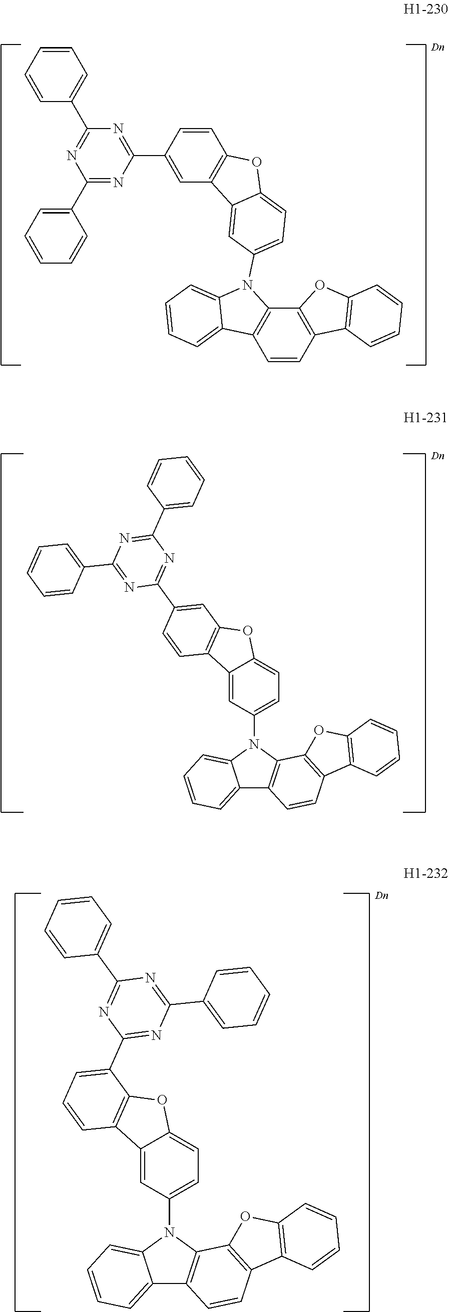

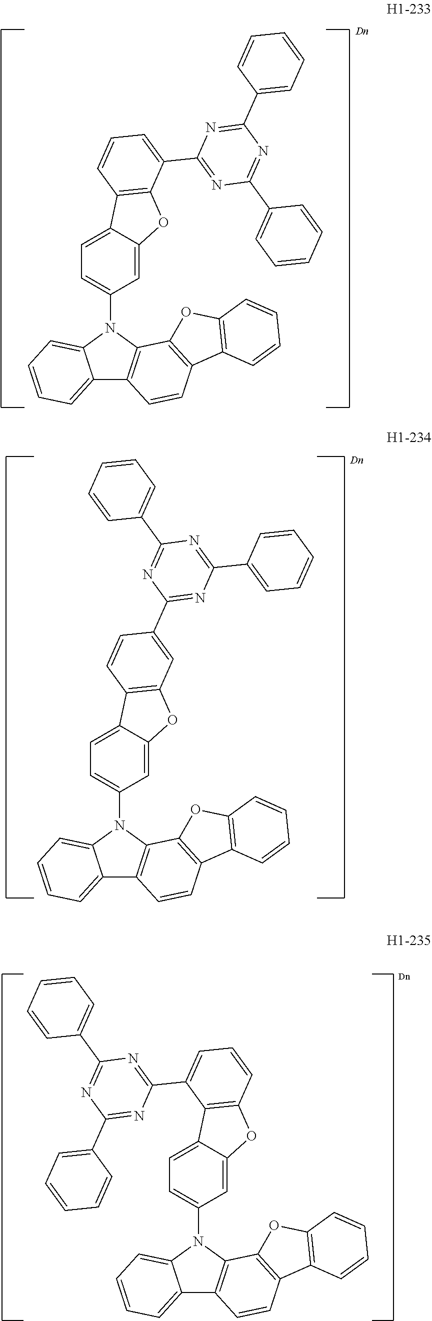

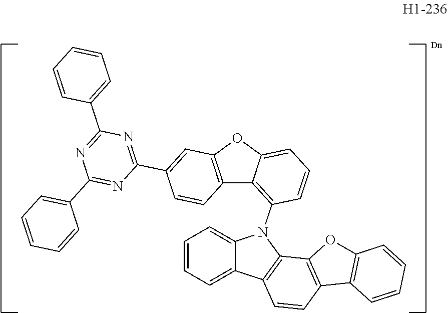

The compound represented by formula 2 may be exemplified as the following compounds, but is not limited thereto.

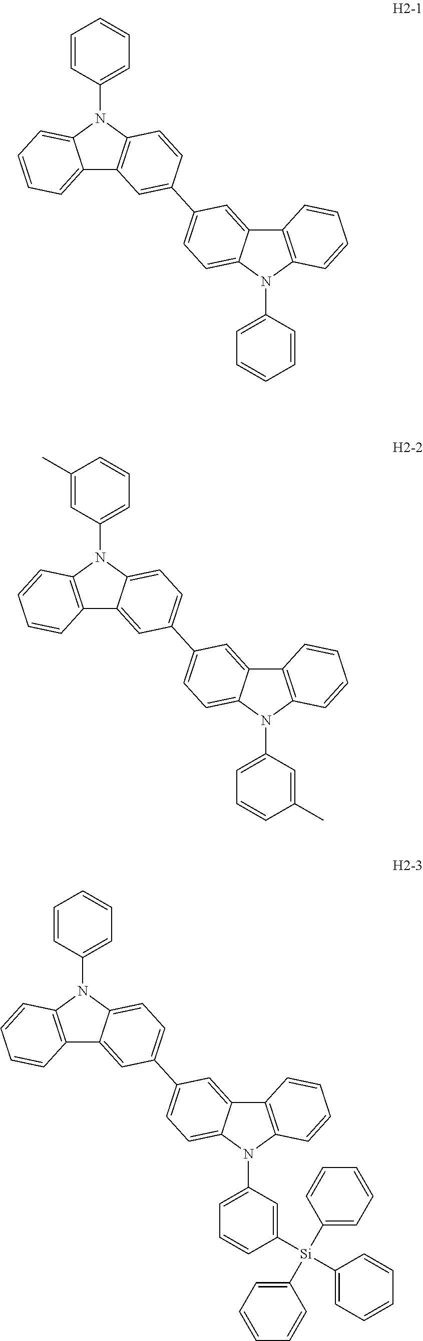 [SMILES: c1ccc(-n2c3ccccc3c3cc(-c4ccc5c(c4)c4ccccc4n5-c4ccccc4)ccc32)cc1 ; Cc1cccc(-n2c3ccccc3c3cc(-c4ccc5c(c4)c4ccccc4n5-c4cccc(C)c4)ccc32)c1 ; c1ccc(-n2c3ccccc3c3cc(-c4ccc5c(c4)c4ccccc4n5-c4cccc([Si](c5ccccc5)(c5ccccc5)c5ccccc5)c4)ccc32)cc1]

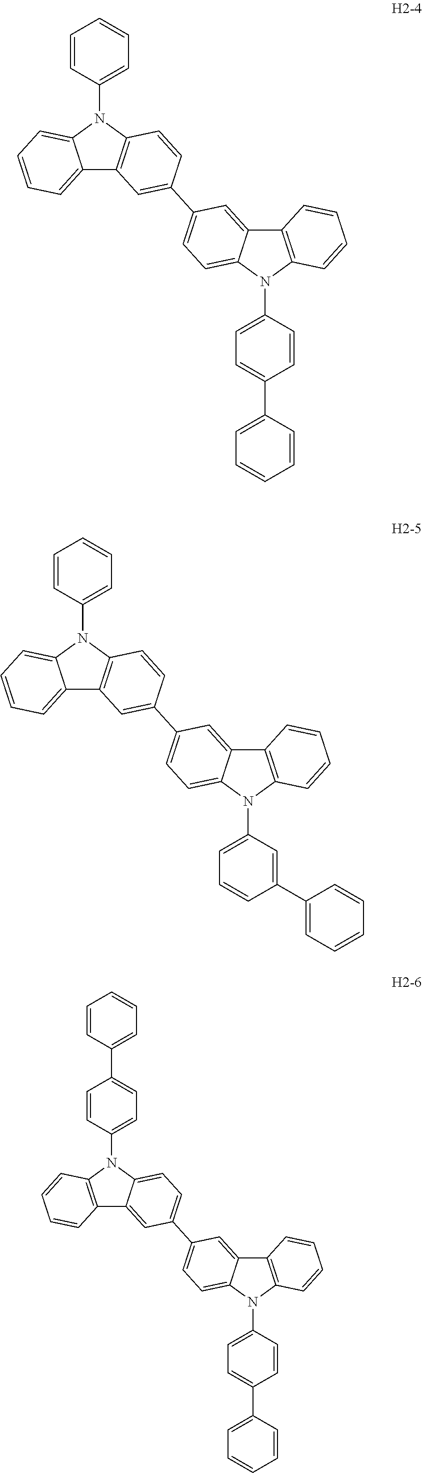 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

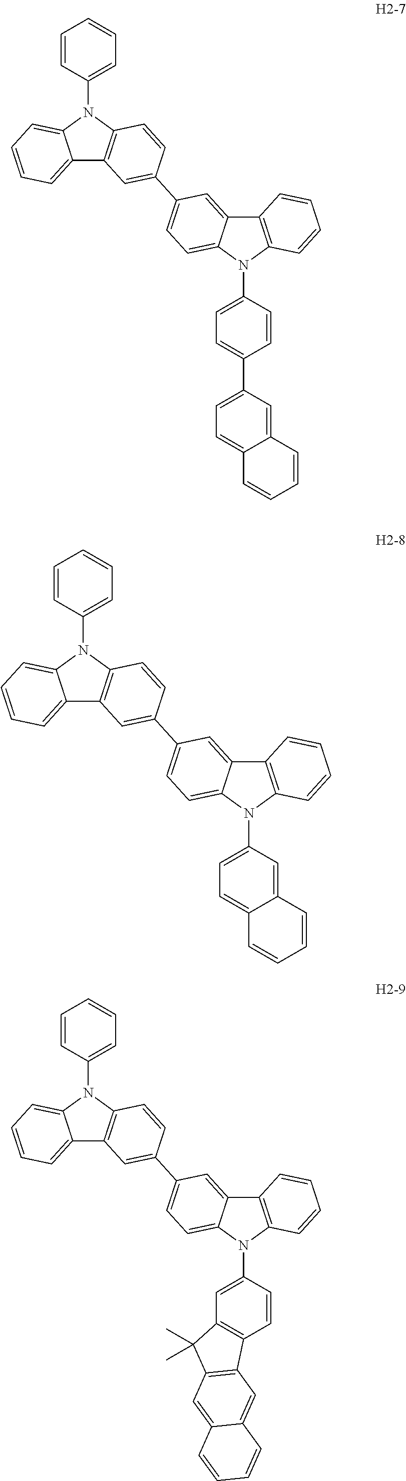 [SMILES: c1ccc(-n2c3ccccc3c3cc(-c4ccc5c(c4)c4ccccc4n5-c4ccc(-c5ccc6ccccc6c5)cc4)ccc32)cc1 ; c1ccc(-n2c3ccccc3c3cc(-c4ccc5c(c4)c4ccccc4n5-c4ccc5ccccc5c4)ccc32)cc1 ; CC1(C)c2cc(-n3c4ccccc4c4cc(-c5ccc6c(c5)c5ccccc5n6-c5ccccc5)ccc43)ccc2-c2cc3ccccc3cc21]

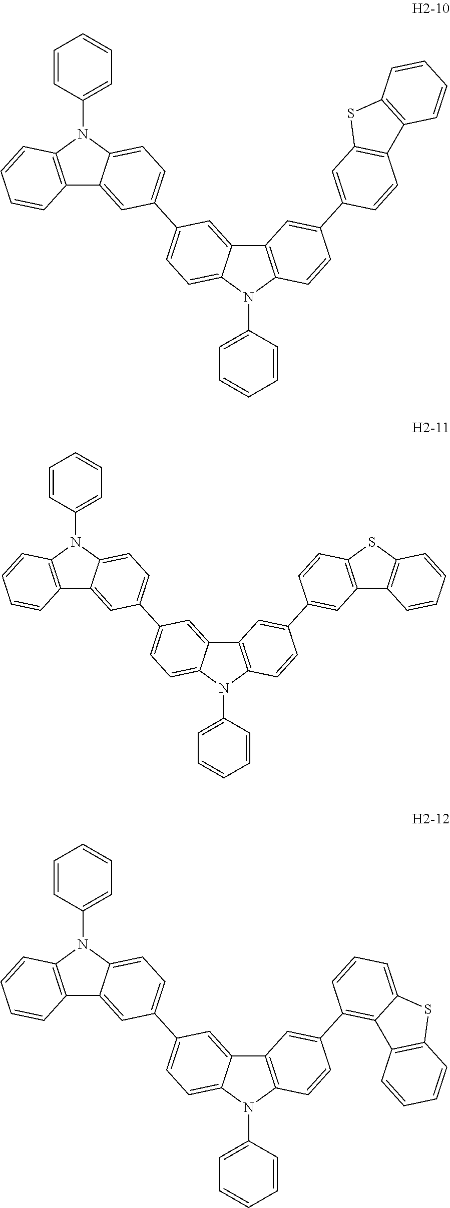 [SMILES: c1ccc(-n2c3ccccc3c3cc(-c4ccc5c(c4)c4cc(-c6ccc7c(c6)sc6ccccc67)ccc4n5-c4ccccc4)ccc32)cc1 ; c1ccc(-n2c3ccccc3c3cc(-c4ccc5c(c4)c4cc(-c6ccc7sc8ccccc8c7c6)ccc4n5-c4ccccc4)ccc32)cc1 ; c1ccc(-n2c3ccccc3c3cc(-c4ccc5c(c4)c4cc(-c6cccc7sc8ccccc8c67)ccc4n5-c4ccccc4)ccc32)cc1]

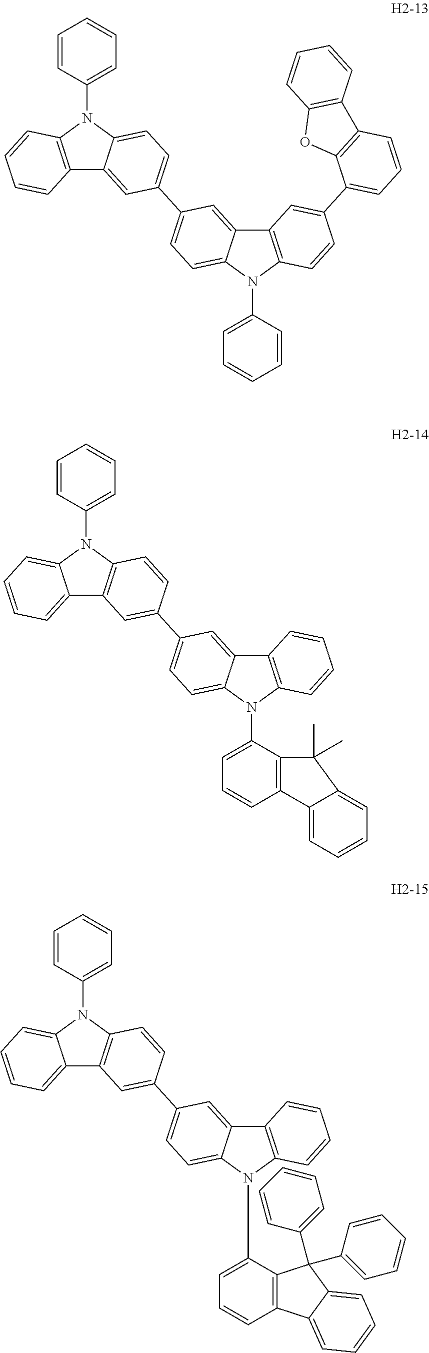 [SMILES: c1ccc(-n2c3ccccc3c3cc(-c4ccc5c(c4)c4cc(-c6cccc7c6oc6ccccc67)ccc4n5-c4ccccc4)ccc32)cc1 ; CC1(C)c2ccccc2-c2cccc(-n3c4ccccc4c4cc(-c5ccc6c(c5)c5ccccc5n6-c5ccccc5)ccc43)c21 ; c1ccc(-n2c3ccccc3c3cc(-c4ccc5c(c4)c4ccccc4n5-c4cccc5c4C(c4ccccc4)(c4ccccc4)c4ccccc4-5)ccc32)cc1]

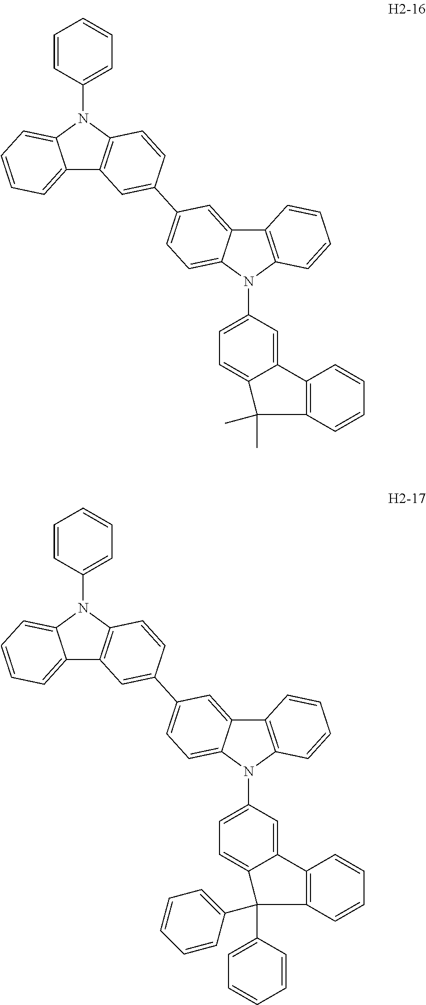 [SMILES: CC1(C)c2ccccc2-c2cc(-n3c4ccccc4c4cc(-c5ccc6c(c5)c5ccccc5n6-c5ccccc5)ccc43)ccc21 ; c1ccc(-n2c3ccccc3c3cc(-c4ccc5c(c4)c4ccccc4n5-c4ccc5c(c4)-c4ccccc4C5(c4ccccc4)c4ccccc4)ccc32)cc1]

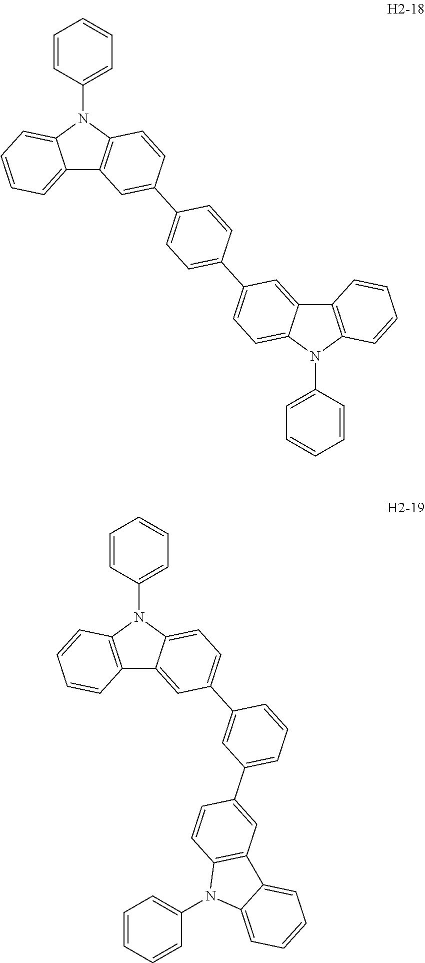 [SMILES: c1ccc(-n2c3ccccc3c3cc(-c4ccc(-c5ccc6c(c5)c5ccccc5n6-c5ccccc5)cc4)ccc32)cc1 ; c1ccc(-n2c3ccccc3c3cc(-c4cccc(-c5ccc6c(c5)c5ccccc5n6-c5ccccc5)c4)ccc32)cc1]

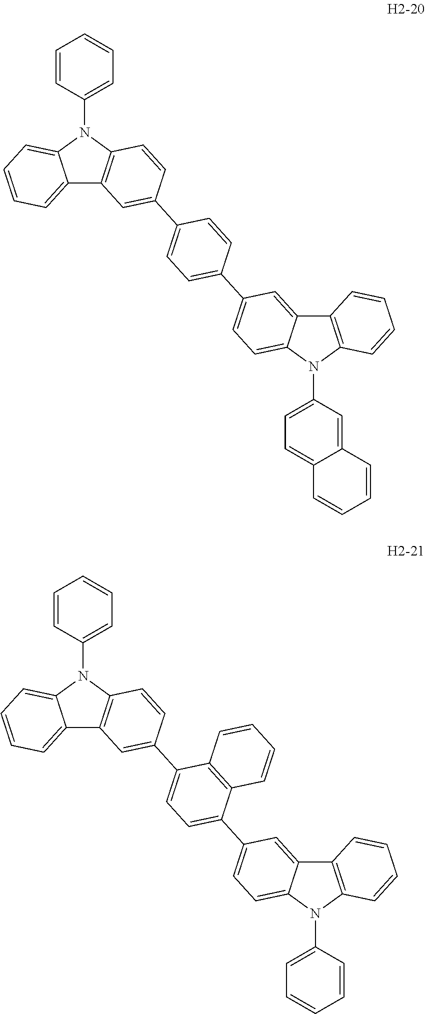 [SMILES: c1ccc(-n2c3ccccc3c3cc(-c4ccc(-c5ccc6c(c5)c5ccccc5n6-c5ccc6ccccc6c5)cc4)ccc32)cc1 ; c1ccc(-n2c3ccccc3c3cc(-c4ccc(-c5ccc6c(c5)c5ccccc5n6-c5ccccc5)c5ccccc45)ccc32)cc1]

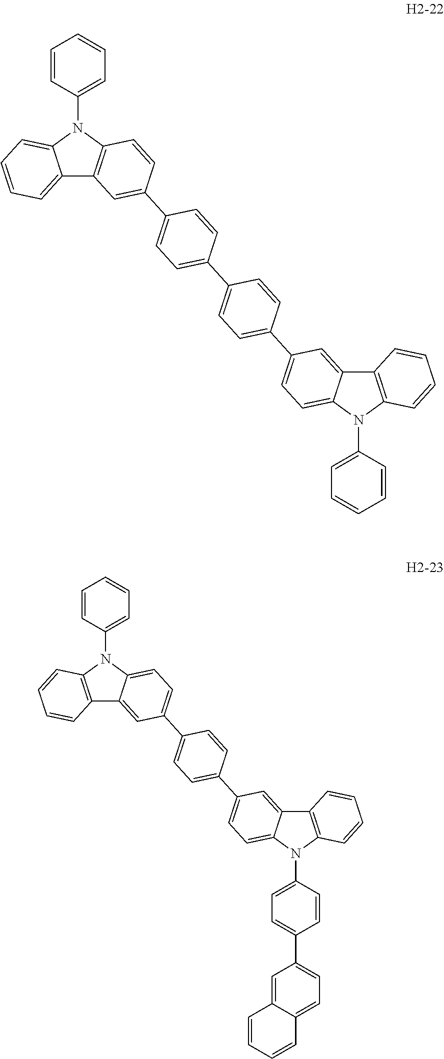 [SMILES: c1ccc(-n2c3ccccc3c3cc(-c4ccc(-c5ccc(-c6ccc7c(c6)c6ccccc6n7-c6ccccc6)cc5)cc4)ccc32)cc1 ; c1ccc(-n2c3ccccc3c3cc(-c4ccc(-c5ccc6c(c5)c5ccccc5n6-c5ccc(-c6ccc7ccccc7c6)cc5)cc4)ccc32)cc1]

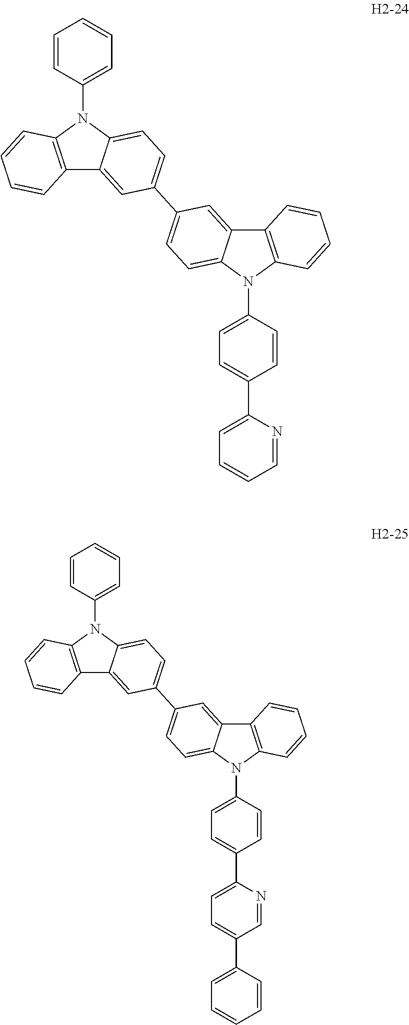 [SMILES: c1ccc(-n2c3ccccc3c3cc(-c4ccc5c(c4)c4ccccc4n5-c4ccc(-c5ccccn5)cc4)ccc32)cc1 ; c1ccc(-c2ccc(-c3ccc(-n4c5ccccc5c5cc(-c6ccc7c(c6)c6ccccc6n7-c6ccccc6)ccc54)cc3)nc2)cc1]

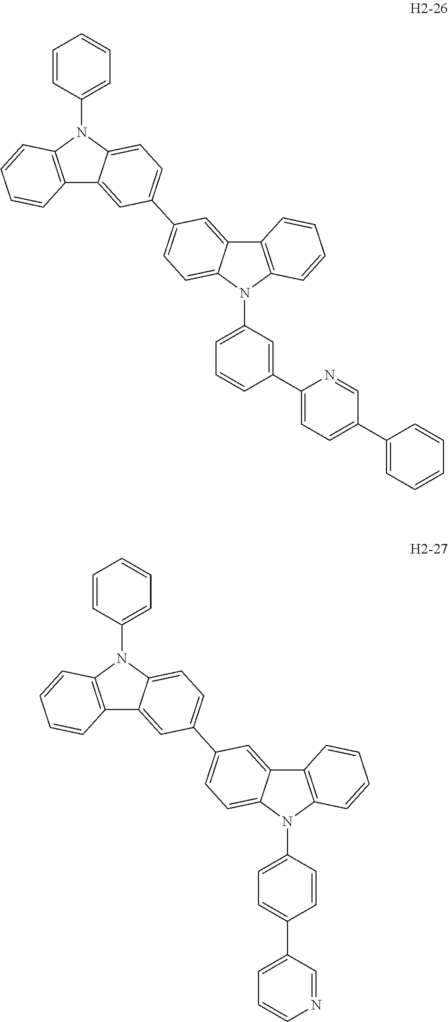 [SMILES: c1ccc(-c2ccc(-c3cccc(-n4c5ccccc5c5cc(-c6ccc7c(c6)c6ccccc6n7-c6ccccc6)ccc54)c3)nc2)cc1 ; c1ccc(-n2c3ccccc3c3cc(-c4ccc5c(c4)c4ccccc4n5-c4ccc(-c5cccnc5)cc4)ccc32)cc1]

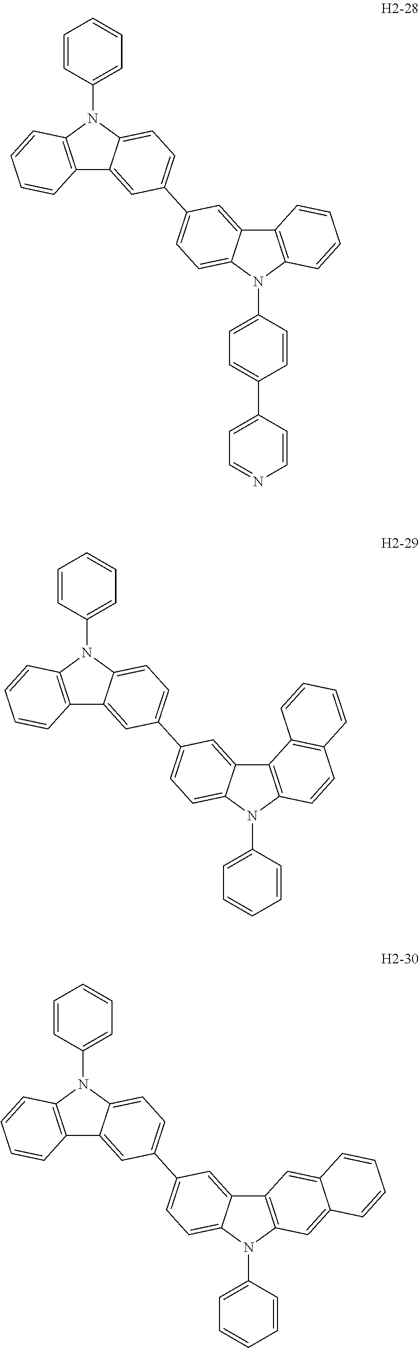 [SMILES: c1ccc(-n2c3ccccc3c3cc(-c4ccc5c(c4)c4ccccc4n5-c4ccc(-c5ccncc5)cc4)ccc32)cc1 ; c1ccc(-n2c3ccccc3c3cc(-c4ccc5c(c4)c4c6ccccc6ccc4n5-c4ccccc4)ccc32)cc1 ; c1ccc(-n2c3ccccc3c3cc(-c4ccc5c(c4)c4cc6ccccc6cc4n5-c4ccccc4)ccc32)cc1]

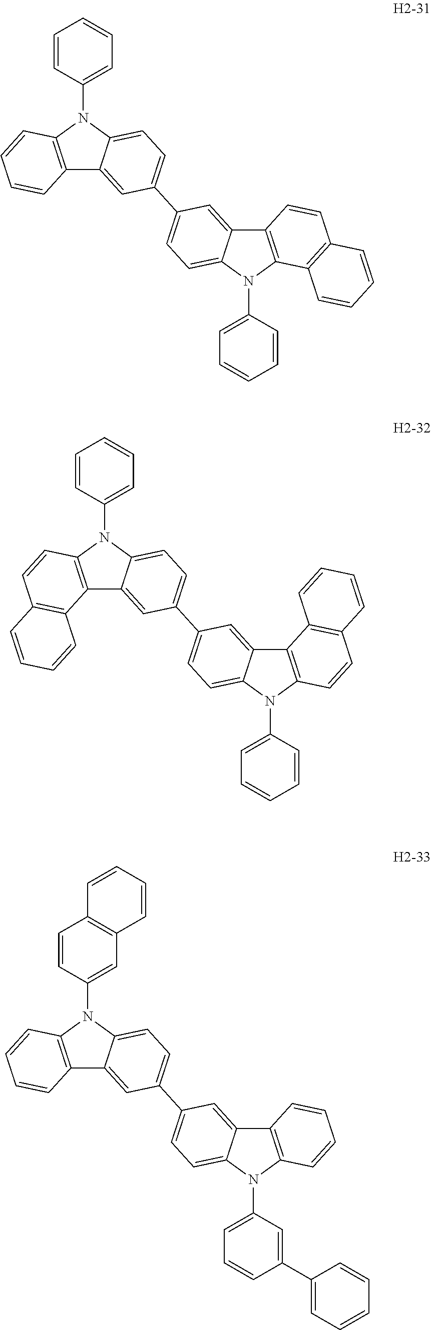 [SMILES: c1ccc(-n2c3ccccc3c3cc(-c4ccc5c(c4)c4ccc6ccccc6c4n5-c4ccccc4)ccc32)cc1 ; c1ccc(-n2c3ccc(-c4ccc5c(c4)c4c6ccccc6ccc4n5-c4ccccc4)cc3c3c4ccccc4ccc32)cc1 ; c1ccc(-c2cccc(-n3c4ccccc4c4cc(-c5ccc6c(c5)c5ccccc5n6-c5ccc6ccccc6c5)ccc43)c2)cc1]

The combination of at least one of compounds H1-1 to H1-236 and at least one of compounds H2-1 to H2-33 may be used in an organic electroluminescent device.

The present disclosure provides an organic electroluminescent compound represented by formula 1, wherein n represents an integer of 5 to 50. According to one embodiment of the present disclosure, the compound may be at least one of compounds H1-1 to H1-236, wherein n, each independently, represents an integer of 5 to 50. According to another embodiment of the present disclosure, at least one of compounds H1-1 to H1-236 (wherein n represents an integer of 5 to 50) may be used in an organic electroluminescent device. The present disclosure may provide an organic electroluminescent device comprising the organic electroluminescent compound, in which the organic electroluminescent compound may be comprised in a light-emitting layer.

The non-deuterated analogues of the compound represented by formula 1 can be prepared by known coupling and substitution reactions. Also, the compound of formula 1 may be prepared in a similar manner by using deuterated precursor materials, or more generally may be prepared by treating the non-deuterated compound with a deuterated solvent or D6-benzene in the presence of an HID exchange catalyst such as a Lewis acid, e.g., aluminum trichloride or ethyl aluminum chloride, a trifluoromethanesulfonic acid, or a trifluoromethanesulfonic acid-D. In addition, the degree of deuteration can be controlled by changing the reaction conditions such as the reaction temperature. For example, the number of n in formula 1 can be controlled by adjusting the reaction temperature and time, the equivalent of the acid, etc.

The compound represented by formula 1 according to the present disclosure may be produced by a synthetic method known to one skilled in the art, and for example, by referring to Korean Patent No. 1427457 (published on Aug. 8, 2014), Korean Patent Application Laying-Open No. 2012-0101029 (published on Sep. 12, 2012), etc., or according to the following reaction schemes 1 to 3, but is not limited thereto.

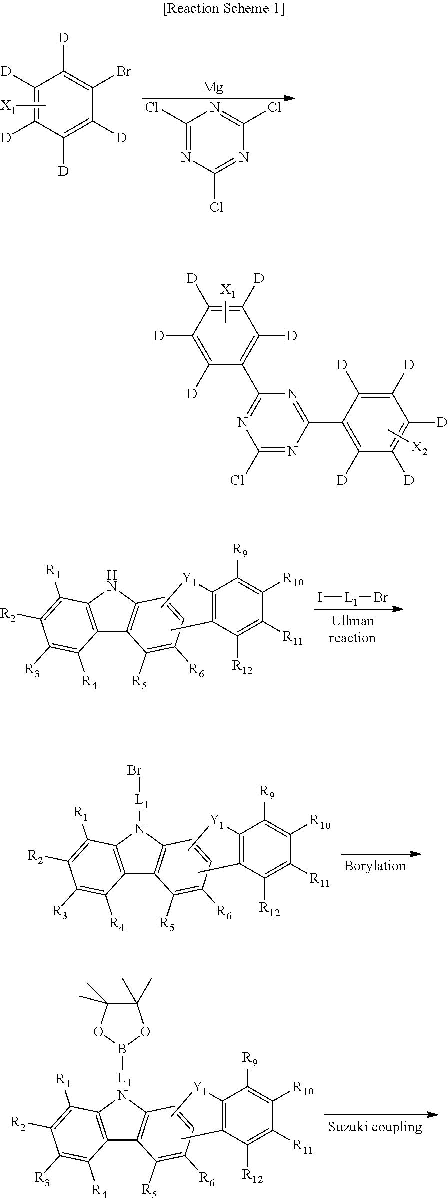

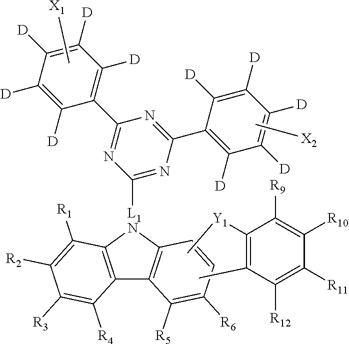

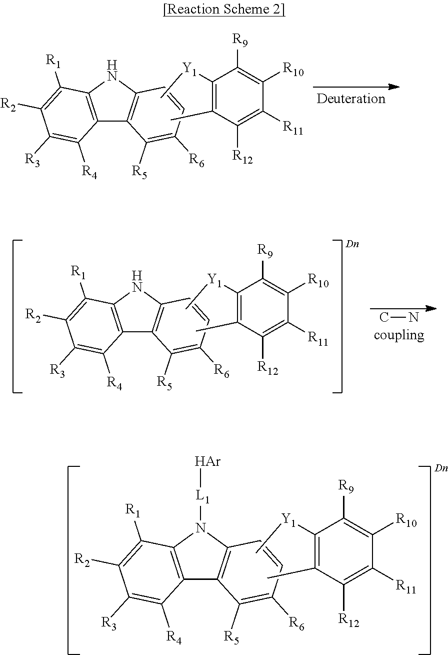

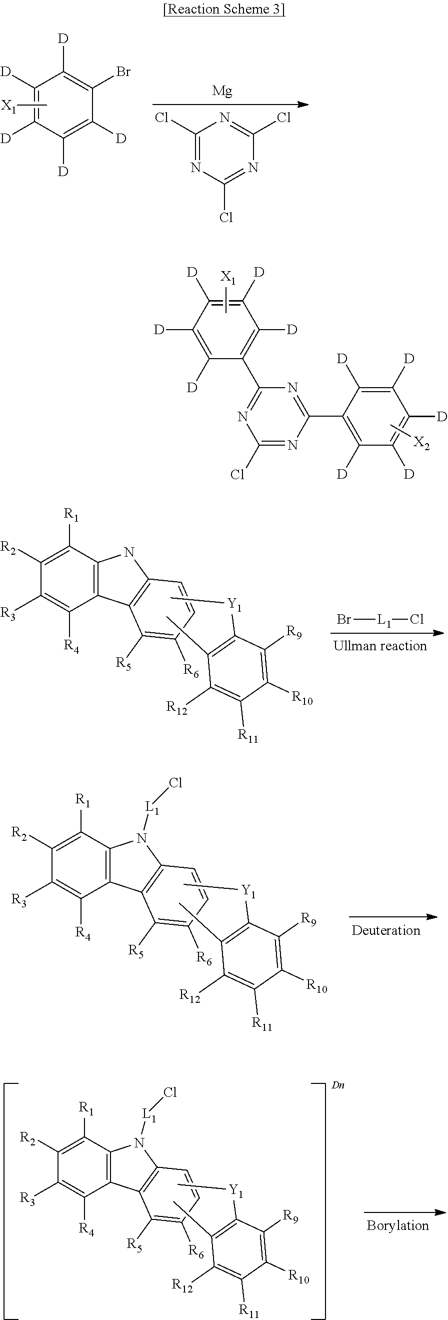

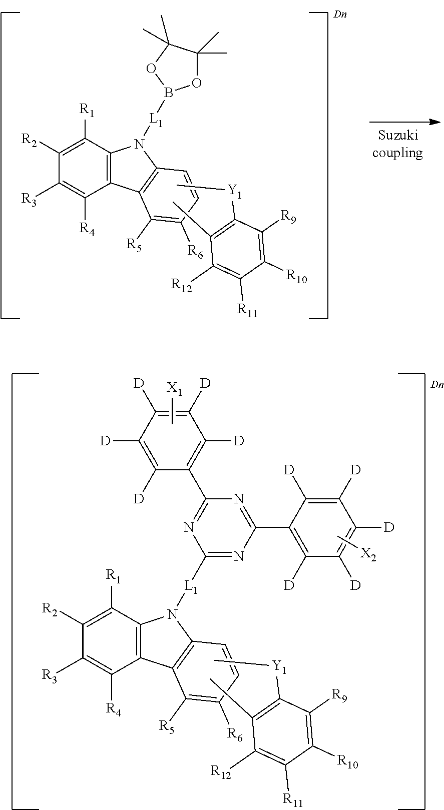

In reaction schemes 1 to 3, R₁ to R₆, R₉ to R₁₂, Y₁, L₁, HAr, and Dn are as defined in formula 1; and X₁ and X₂ represent that deuterium may be replaced with the substituents defined in the present disclosure.

The compound represented by formula 2 according to the present disclosure may be produced by a synthetic method known to one skilled in the art, and for example, by referring to Japanese Patent No. 3139321 (published on Feb. 26, 2001), Korean Patent Application Laying-Open No. 2010-0079458 (published on Jul. 8, 2010), Korean Patent No. 1170666 (published on Aug. 7, 2012), Korean Patent Application Laying-Open No. 2012-0085827 (published on Aug. 1, 2012), Korean Patent Application Laying-Open No, 2014-0037814 (published on Mar. 27, 2014), International Patent Publication No. WO 2012/153725 (published on Nov. 15, 2012), Korean Patent Application Laying-Open No. 2013-009614 (published on Jan. 23, 2013), International Patent Publication No. WO 2013/084881 (published on Jun. 13, 2013), International Patent Publication No, WO 2013/146117 (published on Oct. 3, 2013), International Patent Publication No, WO 2013/146942 (published on Oct. 3, 2013), International Patent Publication No. WO 2014/017484 (published on Jan. 30, 2014), etc., but is not limited thereto.

Although illustrative synthesis examples of the compounds represented by formulas 1 and 2 of the present disclosure are described above, one skilled in the art will be able to readily understand that all of them are based on a Buchwald-Hartwig cross-coupling reaction, an N-acylation reaction, a Miyaura borylation reaction, a Suzuki cross-coupling reaction, a Pd(II)-catalyzed oxidative cyclization reaction, a Heck reaction, a Cyclic Dehydration reaction, an SN; substitution reaction, an SN₂ substitution reaction, a Phosphine-mediated reductive cyclization reaction, a Ullmann reaction, a Wittig reaction, etc., and the reactions above proceed even when substituents which are defined in formulas 1 and 2 above, but are not specified in the specific synthesis examples, are bonded.

The organic electroluminescent device according to the present disclosure may comprise an anode, a cathode, and at least one organic layer between the anode and cathode, in which the organic layer may comprise a plurality of organic electroluminescent materials, including the compound represented by formula 1 as the first organic electroluminescent material, and the compound represented by formula 2 as the second organic electroluminescent material. According to one embodiment of the present disclosure, the organic electroluminescent device according to the present disclosure may comprise an anode, a cathode, and at least one light-emitting layer between the anode and cathode, in which at least one layer of the light-emitting layers may comprise the compound represented by formula 1 and the compound represented by formula 2, preferably a plurality of host materials of the present disclosure.

Herein, the electrode may be a transflective electrode or a reflective electrode, and may be a top emission type, a bottom emission type, or a both-sides emission type, depending on the materials. In addition, the hole injection layer may be further doped with a p-dopant, and the electron injection layer may be further doped with an n-dopant.

The light-emitting layer includes a host and a dopant, in which the host includes a plurality of host materials, and the compound represented by formula 1 may be included as the first host compound of the plurality of host materials, and the compound represented by formula 2 may be included as the second host compound of the plurality of host materials. The weight ratio of the first host compound and the second host compound is about 1:99 to about 99:1, preferably about 10:90 to about 90:10, more preferably about 30:70 to about 70:30, even more preferably about 40:60 to about 60:40, and still more preferably about 50:50. When at least two materials are comprised in one layer, they may be mixture-evaporated to form a layer or may be separately co-evaporated at the same time to form a layer.

In the present disclosure; the light-emitting layer is a layer from which light is emitted, and may be a single layer or a multi-layer of which two or more layers are stacked. AH of the first host material and the second host material may be included in one layer, or the first host material and the second host material may be included in respective different light-emitting layers. According to one embodiment of the present disclosure, the doping concentration of the dopant compound with respect to the host compound in the light-emitting layer may be less than 20 wt %.

The present disclosure may comprise a hole transport zone between an anode and a light-emitting layer, and the hole transport zone may comprise at least one of a hole injection layer, a hole transport layer, a hole auxiliary layer, a light-emitting auxiliary layer and an electron blocking layer. The hole injection layer, the hole transport layer, the hole auxiliary layer, the light-emitting auxiliary layer and the electron blocking layer, respectively, may be a single layer or multi-layers in which two or more layers are stacked. The hole injection layer may be multi-layers in order to lower the hole injection barrier (or hole injection voltage) from the anode to the hole transport layer or the electron blocking layer, wherein two compounds may be used simultaneously in each of the multi-layers. The electron blocking layer may be placed between the hole transport layer (or the hole injection layer) and the light-emitting layer, and can confine the excitons within the light-emitting layer by blocking the overflow of electrons from the light-emitting layer to prevent a light-emitting leakage.

In addition, the hole transport zone may comprise a p-doped hole injection layer, a hole transport layer, and a light-emitting auxiliary layer. The p-doped hole injection layer refers to a hole injection layer doped with a p-dopant. The p-dopant is a material that lets a layer have a p-semiconductor property. The p-semiconductor property refers to the property of a material to inject or transport holes to a HOMO (highest occupied molecular orbital) energy level, i.e., the property of a material having high hole conductivity.

The present disclosure may comprise an electron transport zone between a light-emitting layer and a cathode, and the electron transport zone may comprise at least one of a hole blocking layer, an electron transport layer, an electron buffer layer, and an electron injection layer. The hole blocking layer, the electron transport layer, the electron buffer layer, and the electron injection layer, respectively, may be a single layer or multi-layers in which two or more layers are stacked. The electron injection layer may be further doped with an n-dopant. The electron buffer layer may be multilayers in order to control the electron injection and improve the interfacial properties between the light-emitting layer and the electron injection layer, wherein each of the multilayers may use two compounds simultaneously. The hole blocking layer or the electron transport layer may also be multilayers, wherein each of the multilayers may use a plurality of compounds. According to one embodiment of the present disclosure, at least one layer, preferably the electron buffer layer, of the electron transport zone may comprise the compound represented by formula 1.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as a hole auxiliary layer or an electron blocking layer. The light-emitting auxiliary layer, the hole auxiliary layer and the electron blocking layer may have an effect of improving the efficiency and/or the lifetime of the organic electroluminescent device.

The dopant comprised in the organic electroluminescent device of the present disclosure may be at least one phosphorescent or fluorescent dopant, and is preferably a phosphorescent dopant. The phosphorescent dopant material applied to the organic electroluminescent device of the present disclosure is not particularly limited, but may be preferably selected from the metallated complex compounds of iridium (Ir), osmium (Os), copper (Cu), and platinum (Pt), more preferably selected from ortho-metallated complex compounds of iridium (Ir), osmium (Os), copper (Cu), and platinum (Pt), and even more preferably ortho-metallated iridium complex compounds.

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used.

When using a wet film-forming method, a thin film can be formed by dissolving or diffusing materials forming each layer into any suitable solvent such as ethanol, chloroform, tetrahydrofuran, dioxane, etc. The solvent can be any one where the materials forming each layer can be dissolved or diffused, and where there are no problems in film-formation capability.

In addition, the compound represented by formula 1 and the compound represented by formula 2 may be film-formed by the above-listed methods, commonly by a co-evaporation process or a mixture-evaporation process. The co-evaporation is a mixed deposition method in which two or more materials are placed in a respective individual crucible source and a current is applied to both cells at the same time to evaporate the materials. The mixture-evaporation is a mixed deposition method in which two or more materials are mixed in one crucible source before evaporating them, and a current is applied to the cell to evaporate the materials.

The organic electroluminescent material according to the present disclosure may be used as a light-emitting material for a white organic light-emitting device. The white organic light-emitting device has been suggested to have various structures such as a side-by-side structure or a stacking structure depending on the arrangement of R (red), G (green) or YG (yellow green), and B (blue) light-emitting parts, or color conversion material (CCM) method, etc. The present disclosure may also be applied to the white organic light-emitting device. In addition, the organic electroluminescent material according to the present disclosure may also be used in an organic electroluminescent device comprising a quantum dot (QD).

The present disclosure may provide a display system comprising the plurality of host materials of the present disclosure. In addition, by using the organic electroluminescent device of the present disclosure, it is possible to manufacture a display system or a lighting system. Specifically, it is possible to produce a display system, for example, a display system for smart phones, tablets, notebooks, PCs, TVs, or cars; or a lighting system, for example an outdoor or indoor lighting system, by using the organic electroluminescent device of the present disclosure.

Hereinafter, the preparation method of the compounds according to the present disclosure and the properties thereof will be explained in detail with reference to the representative compounds of the present disclosure. However, the present disclosure is not limited by the following examples.

### EXAMPLE 1: PREPARATION OF COMPOUND H1-211

Synthesis of Compound 1-1

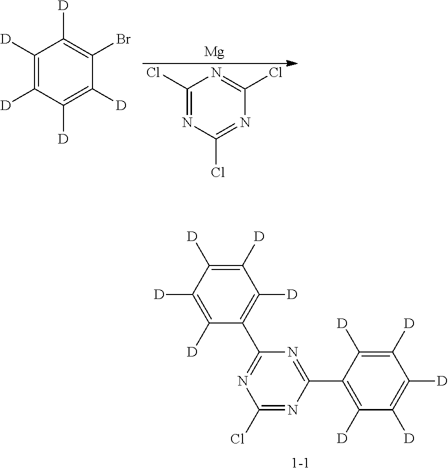

In a flask, Mg (3.29 g, 135.56 mmol), tetrahydrofuran (THF) 60 mL, and I₂ (0.137 g, 0.54 mmol) were stirred, Bromobenzene-D5 (21.9 g, 135.56 mmol) was slowly added to the mixture, heated to 75° C., and cooled to room temperature after 30 minutes to produce a Grignard reagent. 2,4,6-trichloro-1,3,5-triazine (10 g, 54.22 mmol) was dissolved in 120 mL of THF, and the mixture was cooled to 0° C. and the Grignard reagent produced was slowly added thereto. After stirring at room temperature for 12 hours, an aqueous NH₄Cl solution was added to the mixture. An organic layer was extracted with ethyl acetate, and the residual moisture was removed using magnesium sulfate. The residue was distilled under reduced pressure, and separated by column chromatography to obtain compound 1-1 (9.5 g, yield: 63.08%).

Synthesis of Compound 1-2

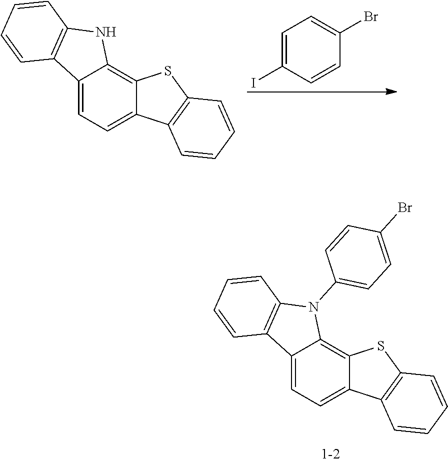

12H-benzo[4,5]thieno[2,3-a]carbazole (25 g, 91.45 mmol), 4-bromoiodobenzene (51.58 g, 182.9 mmol), Cul (13.9 g, 73.16 mmol), 1000 mL of toluene, Cs₂CO₃ (74.5 g, 228.6 mmol), and ethylenediamine (12.2 mL, 182.9 mmol) were added to a flask. The mixture was heated to 155° C., and cooled to room temperature after 5 hours. Distilled water was added to the mixture, and an organic layer was extracted with ethyl acetate. After removing the residual moisture with magnesium sulfate, the residue was distilled under reduced pressure, and separated by column chromatography to obtain compound 1-2 (18.5 g, yield: 47.23%).

Synthesis of Compound 1-3

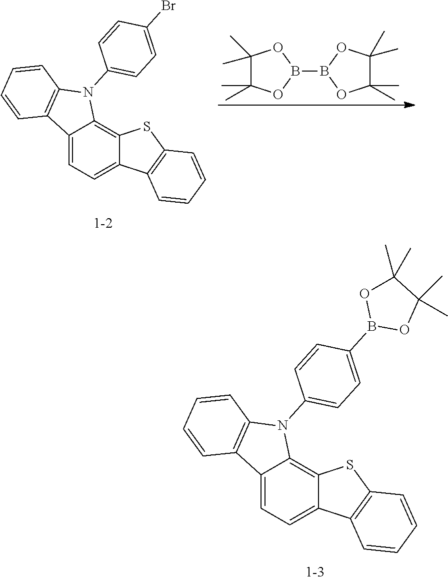

Compound 1-2 (18.5 g, 43.18 mmol), bis(pinacolato)diboron (14.25 g, 56.14 mmol), PdCl₂(PPh₃)₂ (1.5 g, 2.16 mmol), KOAc (8.5 g, 86.37 mmol), and 800 mL of 1,4-dioxane were added to a flask. The mixture was heated to 145° C., and cooled to room temperature after 4 hours. Distilled water was added to the mixture, and an organic layer was extracted with ethyl acetate. After removing the residual moisture with magnesium sulfate, the residue was distilled under reduced pressure, and separated by column chromatography to obtain compound 1-3 (14 g, yield: 68.22%).

Synthesis of Compound H1-211

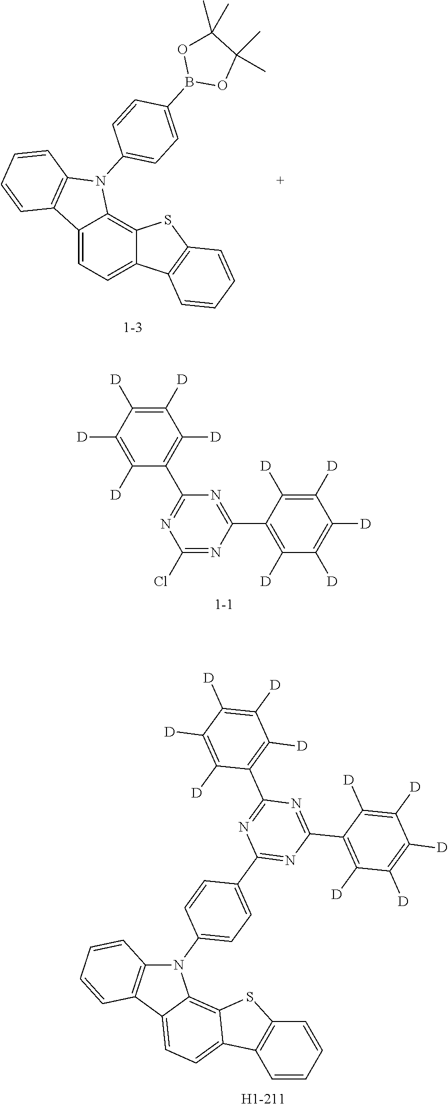 [SMILES: CC1(C)OB(c2ccc(-n3c4ccccc4c4ccc5c6ccccc6sc5c43)cc2)OC1(C)C ; [2H]c1c([2H])c([2H])c(-c2nc(Cl)nc(-c3c([2H])c([2H])c([2H])c([2H])c3[2H])n2)c([2H])c1[2H] ; [2H]c1c([2H])c([2H])c(-c2nc(-c3ccc(-n4c5ccccc5c5ccc6c7ccccc7sc6c54)cc3)nc(-c3c([2H])c([2H])c([2H])c([2H])c3[2H])n2)c([2H])c1[2H]]

Compound 1-3 (14 g, 29.45 mmol), compound 1-1 (8.9 g, 32.4 mmol). Pd(PPh₃)₄ (1.7 g, 1.47 mmol), K₂CO₃ (8.1 g, 58.89 mmol), 400 mL of toluene, 60 mL of distilled water, and 40 mL of ethanol were added to a flask. The mixture was heated to 140° C., and cooled to room temperature after 5 hours. Distilled water was added to the mixture, and an organic layer was extracted with ethyl acetate. After removing the residual moisture with magnesium sulfate, the residue was distilled under reduced pressure, and separated by column chromatography to obtain compound H1-211 (10.5 g, yield: 60.3%)

| | MW | M.P. |
|---|---|---|
| H1-211 | 590 | 335° C. |

### EXAMPLE 2: PREPARATION OF COMPOUND H1-212

Synthesis of Compound 2-1

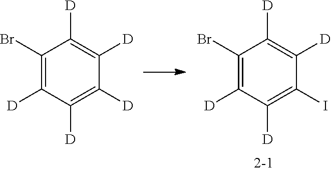 [SMILES: [2H]c1c([2H])c([2H])c(Br)c([2H])c1[2H] ; [2H]c1c([2H])c(I)c([2H])c([2H])c1Br]

5-bromobenzene-D5 (36 g, 222.16 mmol), 216 mL of dichloromethane, I₂ (45 g, 177.7 mmol), 108 mL of acetic acid, and 3.5 mL of sulfuric acid were added to a flask, and the mixture was stirred at 35° C. for 10 minutes. Then, K₂S₂O₈ (18.01 g, 66:65 mmol) was added to the mixture, heated to 45° C., and cooled to room temperature after 4 hours. The reaction solution was slowly added to an aqueous potassium carbonate solution. After neutralizing the reaction solution, an organic layer was extracted with dichloromethane. The organic layer was added to an aqueous sodium thiosulfate solution, and stirred. Thereafter, an organic layer and an aqueous layer were separated. The residual moisture was removed using magnesium sulfate, and the residue was separated by column chromatography to obtain compound 2-1 (27 g, yield: 42.8° M.

Synthesis of Compound 2-2

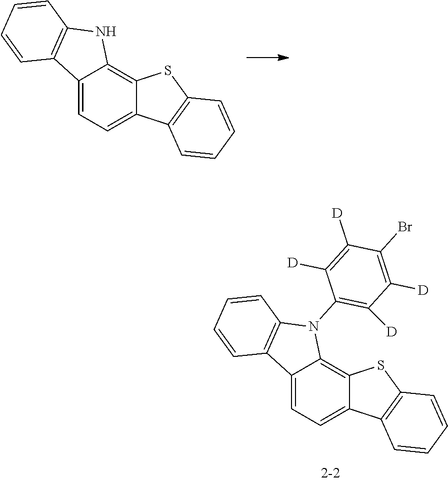 [SMILES: c1ccc2c(c1)[nH]c1c2ccc2c3ccccc3sc21 ; [2H]c1c([2H])c(-n2c3ccccc3c3ccc4c5ccccc5sc4c32)c([2H])c([2H])c1Br]

12H-benzo[4,5]thieno[2,3-a]carbazole (20 g, 73.16 mmol), compound 2-1 (27.29 g, 95.11 mmol), Cul (11.14 g, 58.53 mmol), 700 mL of toluene, Cs₂CO₃ (59.59 g, 182.91 mmol), and ethylenediamine (9.8 mL, 146.3 mmol) were added to a flask. The mixture was heated to 160° C., and cooled to room temperature after 19 hours. Distilled water was added to the mixture, and an organic layer was extracted with ethyl acetate. After removing the residual moisture with magnesium sulfate, the residue was distilled under reduced pressure, and separated by column chromatography to obtain compound 2-2 (18.5 g, yield: 47.23%).

Synthesis of Compound 2-3

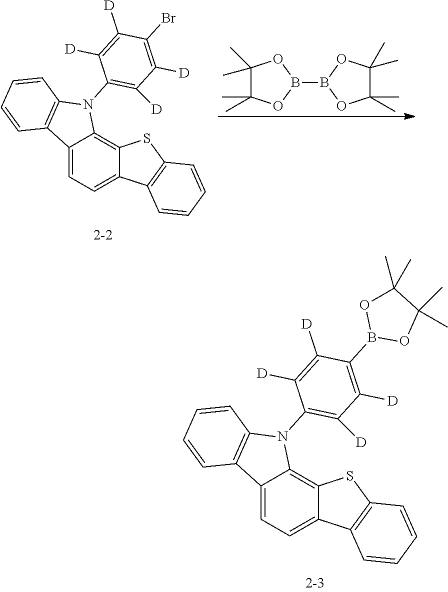

Compound 2-2 (23 g, 53.19 mmol), bis(pinacolato)diboron (17.5 g, 69.15 mmol), PdCl₂(PPh₃)₂ (1.86 g, 2.66 mmol), KOAc (10.46 g, 106.4 mmol), and 900 mL of 1,4-dioxane were added to a flask. The mixture was heated to 145° C., and cooled to room temperature after 5 hours. Distilled water was added to the mixture, and an organic layer was extracted with ethyl acetate. After removing the residual moisture with magnesium sulfate, the residue was distilled under reduced pressure, and separated by column chromatography to obtain compound 2-3 (14 g, yield: 54.9%).

Synthesis of Compound H1-212

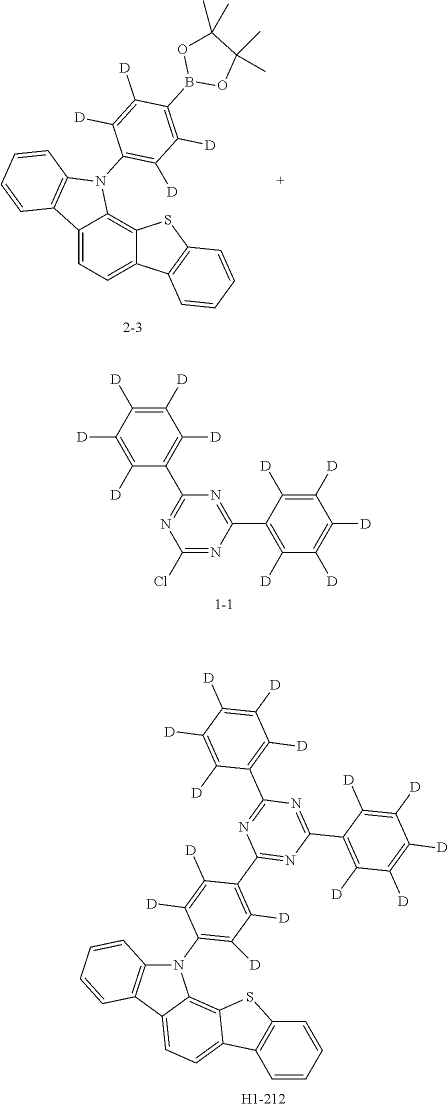 [SMILES: [2H]c1c([2H])c(-n2c3ccccc3c3ccc4c5ccccc5sc4c32)c([2H])c([2H])c1B1OC(C)(C)C(C)(C)O1 ; [2H]c1c([2H])c([2H])c(-c2nc(Cl)nc(-c3c([2H])c([2H])c([2H])c([2H])c3[2H])n2)c([2H])c1[2H] ; [2H]c1c([2H])c([2H])c(-c2nc(-c3c([2H])c([2H])c([2H])c([2H])c3[2H])nc(-c3c([2H])c([2H])c(-n4c5ccccc5c5ccc6c7ccccc7sc6c54)c([2H])c3[2H])n2)c([2H])c1[2H]]

Compound 2-3 (14 g, 29.20 mmol), compound 1-1 (8.9 g, 32.12 mmol), Pd(PPh₃)₄ (1.68 g, 1.46 mmol), K₂CO₃ (8.0 g, 58.40 mmol), 400 mL of toluene, 60 mL of distilled water, and 40 mL of ethanol were added to a flask. The mixture was stirred under reflux, and cooled to room temperature after 5 hours. Distilled water was added to the mixture, and an organic layer was extracted with ethyl acetate. The residue was distilled under reduced pressure, and separated by column chromatography to obtain compound H1-212 (10.5 g, yield: 60.45%).

| | MW | M.P. |
|---|---|---|
| H1-212 | 594 | 335° C. |

### EXAMPLE 3: PREPARATION OF COMPOUND H1-213

Synthesis of Compound 3-1

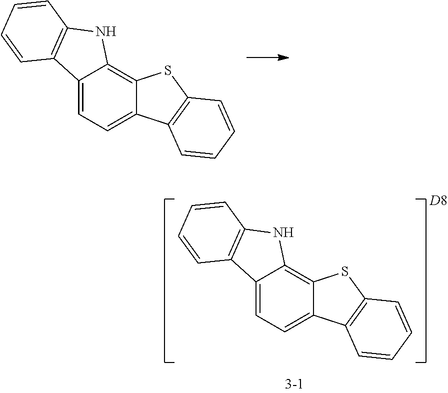

12H-benzo[4,5]thieno[2,3-a]carbazole (20.0 g, 9.0 mmol), and benzene-D6 (1.2 kg, 14.63 mol) were added to a flask, and the mixture was stirred under reflux. Truffle acid (65.88 g, 438.9 mmol) was added to the mixture at 70° C., and cooled to room temperature after 5 hours. 40 mL of D₂O was added to the mixture, and stirred for 10 minutes. The reaction solution was neutralized with an aqueous K₃PO₄ solution, and an organic layer was extracted with ethyl acetate. After removing the residual moisture with magnesium sulfate, the residue was distilled under reduced pressure, and separated by column chromatography to obtain compound 3-1 (15 g, yield: 72.99%).

Synthesis of Compound H1-213

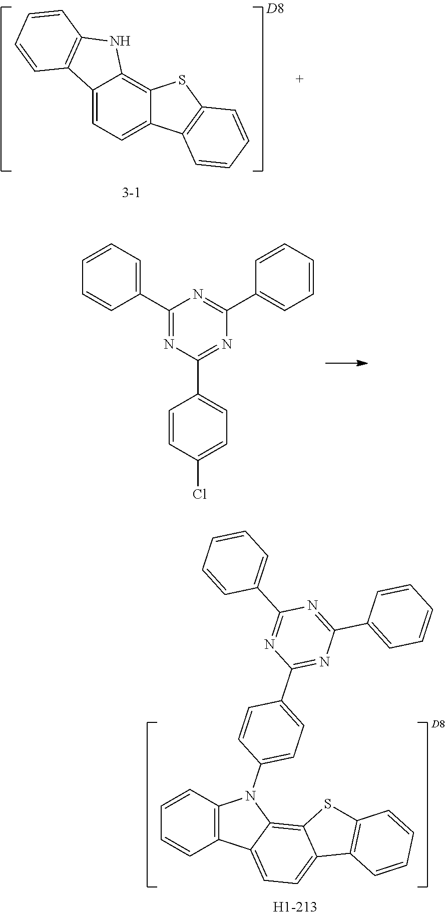

In a flask, compound 3-1 (14 g, 49.8 mmol), 2-(4-chlorophenyi)-4,6-diphenyl-1,3,5-triazine (23.21 g, 59.78 mmol), Pd(OAc)₂ (0.55 g, 2.49 mmol), S-phos (2.04 g, 4.98 mmol), NaOt-Bu (8.6 g, 90.14 mmol), and 500 mL of o-xylene were stirred, and heated to 185° C. for 4 hours. Then, the mixture was cooled to room temperature, and distilled water was added thereto. An organic layer was extracted with ethyl acetate, and distilled under reduced pressure. The resulting solid was separated by column chromatography to obtain compound H1-213 (20.5 g, yield: 70.0%).

| | MW | M.P. |
|---|---|---|
| H1-213 | 588 | 334° C. |

### EXAMPLE 4: PREPARATION OF COMPOUND H1-36

Synthesis of Compound 4-1

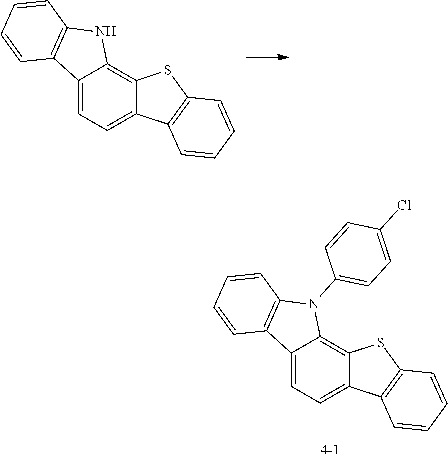 [SMILES: c1ccc2c(c1)[nH]c1c2ccc2c3ccccc3sc21 ; Clc1ccc(-n2c3ccccc3c3ccc4c5ccccc5sc4c32)cc1]

12H-benzo[4,5]thieno[2,3-a]carbazole (20 g, 73.16 mmol), 1-bromo-4-chlorobenzene (42 g, 219.49 mmol), Cul (7 g, 36.58 mmol), 500 mL of toluene, K₃PO₄ (47 g, 219.49 mmol), and ethylenediamine (10 mL, 146.33 mmol) were added to a flask. The mixture was heated to 160° C., and cooled to room temperature after 16 minutes. Distilled water was added to the mixture and an organic layer was extracted with ethyl acetate. After removing the residual moisture with magnesium sulfate, the residue was distilled under reduced pressure, and separated by column chromatography to obtain compound 4-1 (9.3 g, yield: 33.2%).

Synthesis of Compound 4-2

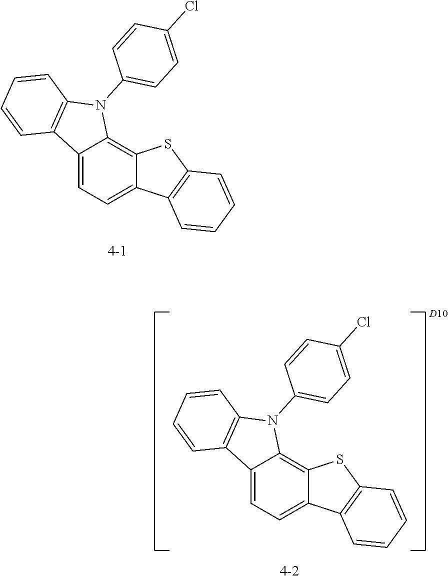

Compound 4-1 (8.8 g, 22.97 mmol), and benzene-D6 (528 mL, 5.49 mol) were added to a flask, and triflic acid (26.4 mL, 293.78 mmol) was then added to the mixture. The mixture was heated to 50° C. for 3 hours, and cooled to room temperature. 8.8 mL of D₂O was added to the mixture, and stirred for 10 minutes. The reaction solution was neutralized with an aqueous K₃PO₄ solution, and an organic layer was extracted with ethyl acetate. After removing the residual moisture with magnesium sulfate, the residue was distilled under reduced pressure, and separated by column chromatography to obtain compound 4-2 (8.2 g, yield: 93.2%).

Synthesis of Compound 4-3

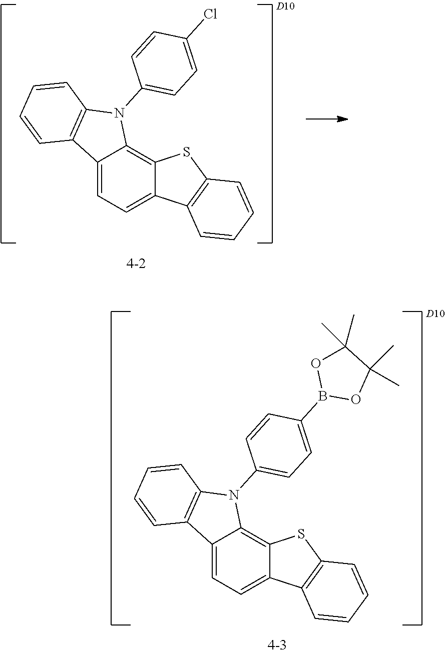

Compound 4-2 (8.2 g, 53.19 mmol), 4,4,4′,4′,5,5,5′,5′-octamethyl-2,2′-bi(1,3,2-dioxaborolane) (7.95 g, 31.29 mmol), Pd₂(dba)₃ (1.0 g, 1.09 mmol), S-Phos (1.0 g, 2.43 mmol), KOAc (5.2 g, 52.98 mmol), and 200 mL of 1,4-dioxane were added to a flask. The mixture was heated to 140° C., and cooled to room temperature after 7 hours. Distilled water was added to the mixture, and an organic layer was extracted with dichloromethane. After removing the residual moisture with magnesium sulfate, the residue was distilled under reduced pressure, and separated by column chromatography to obtain compound 4-3 (8.1 g, yield: 80.1%).

Synthesis of Compound H1-36

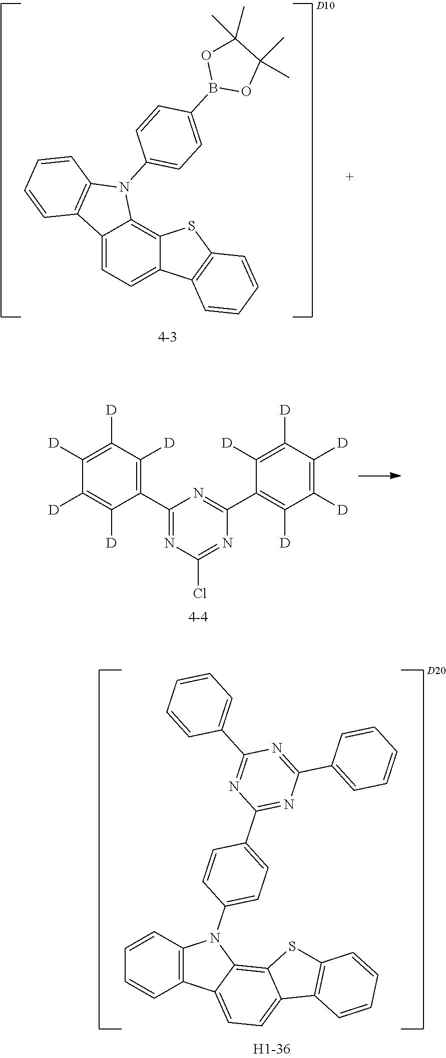

Compound 4-3 (8.1 g, 16.68 mmol), compound 4-4 (4.2 g, 14.59 mmol), PdCl₂(Amphos)₂ (0.6 g, 0.84 mmol), Na₂CO₃ (3.5 g, 33.02 mmol), 63 mL of toluene, 21 mL of distilled water, and Aliquat 336 (0.29 g, 0.73 mmol) were added to a flask. The mixture was stirred under reflux, and cooled to room temperature after 4 hours. Distilled water was added to the mixture, and an organic layer was extracted with ethyl acetate. The organic layer was distilled under reduced pressure, and separated by column chromatography to obtain compound H1-36 (3 g, yield: 34.2%).

Hereinafter, a method of producing an organic electroluminescent device (OLED) according to the present disclosure and the luminous efficiency and lifetime properties thereof will be explained in detail. However, the present disclosure is not limited by the following examples.

### Device Example 1: Producing an OLED Comprising the Host Materials According to the Present Disclosure

An OLED according to the present disclosure was produced. A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropanol. The ITO substrate was then mounted on a substrate holder of a vacuum vapor deposition apparatus, Compound HI-1 shown in Table 2 was introduced into a cell of the vacuum vapor deposition apparatus, and compound HT-1 shown in Table 2 was introduced into another cell of the vacuum vapor deposition apparatus. The two materials were evaporated at different rates, and compound HI-1 was deposited in a doping amount of 3 wt % based on the total amount of compound HI-1 and compound HT-1 to form a hole injection layer having a thickness of 10 nm on the ITO substrate. Next, compound HT-1 was deposited on the hole injection layer to form a first hole transport layer having a thickness of 80 nm on the hole injection layer. Compound HT-2 was then introduced into another cell of the vacuum vapor deposition apparatus and was evaporated by applying an electric current to the cell, thereby forming a second hole transport layer having a thickness of 30 nm on the first hole transport layer. After forming the hole injection layer and the hole transport layers, a light-emitting layer was formed thereon as follows: Compound H1-211 and compound H2-6 shown in Table 1 below were introduced into two cells of the vacuum vapor depositing apparatus as hosts, and compound D-50 was introduced into another cell as a dopant. The two host materials were evaporated at a different rate of 1:2 and the dopant material was simultaneously evaporated at a different rate, and the dopant was deposited in a doping amount of 10 wt % based on the total amount of the hosts and the dopant to form a light-emitting layer having a thickness of 40 nm on the second hole transport layer. Compound ETL-1 and compound EIL-1 were evaporated in a weight ratio of 40:60 to form an electron transport layer having a thickness of 35 nm on the light-emitting layer. After depositing compound EIL-1 as an electron injection layer having a thickness of 2 nm on the electron transport layer, an Al cathode having a thickness of 80 nm was deposited on the electron injection layer by another vacuum vapor deposition apparatus.

Thus, an OLED was produced. All the materials used for producing the OLED were purified by vacuum sublimation at 10⁻⁶ torr.

### Device Example 2: Producing an OLED Comprising the Host Materials According to the Present Disclosure

An OLED was produced in the same manner as in Device Example 1, except that compound H1-212 was used as the first host of the light-emitting layer.

### Device Example 3: Producing an OLED Comprising the Host Materials According to the Present Disclosure

An OLED was produced in the same manner as in Device Example 1, except that compound H1-213 was used as the first host of the light-emitting layer.

### Comparative Example 1: Producing an OLED Comprising a Conventional Compound as a First Host

OLEDs were produced in the same manner as in Device Example 1, except that compound CA was used as the first host of the light-emitting layer.

The driving voltage, luminous efficiency, and light-emitting color at a luminance of 1,000 nit, and the time taken for luminance to decrease from 100% to 85% at a luminance of 20,000 nit (lifetime; T85) of the OLEDs produced in the Device Examples and the Comparative Example are provided in Table 1 below.

TABLE 1

| | | | | Luminous | | Life- |
|---|---|---|---|---|---|---|
| | | | Driving | Effi- | Light- | time |
| | First | Second | Voltage | ciency | Emitting | (T85) |
| | Host | Host | [V] | [cd/A] | Color | [h] |
| Device | H1-211 | H2-6 | 3.1 | 92.2 | Green | 668 |
| Example 1 | | | | | | |
| Device | H1-212 | H2-6 | 3.1 | 93.2 | Green | 786 |
| Example 2 | | | | | | |
| Device | H1-213 | H2-6 | 3.1 | 93.9 | Green | 759 |
| Example 3 | | | | | | |
| Comparative | C-1 | H2-6 | 3.1 | 91.8 | Green | 655 |
| Example 1 | | | | | | |

From Table 1 above; it can be seen that the OLEDs comprising a plurality of host materials according to the present disclosure have excellent luminous properties, and in particular improved lifetime property, compared to the conventional OLEDs. That is, it is confirmed that the lifetime property of the green phosphorescent host can be improved by introducing a deuterated residue(s) into the conventional host material. It is understood that the compound substituted with deuterium lowers the zero point vibration energy and increases bond dissociation energy (BDE), thereby improving the stability of the host. In addition, this can enhance the performance of the host, thereby improving the lifetime property of the host, in particular the green phosphorescent host.

### Device Example 4: Producing a Blue OLED Comprising the Electron Buffer Layer Compound According to the Present Disclosure

A blue OLED according to the present disclosure was produced. A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropanol. The ITO substrate was then mounted on a substrate holder of a vacuum vapor deposition apparatus. Compound HI-1 shown in Table 2 was introduced into a cell of the vacuum vapor deposition apparatus, and compound HT-1 shown in Table 2 was introduced into another cell of the vacuum vapor deposition apparatus. The two materials were evaporated at different rates, and compound HI-1 was deposited in a doping amount of 3 wt % based on the total amount of compound HI-1 and compound HT-1 to form a hole injection layer having a thickness of 10 nm on the ITO substrate. Next, compound HT-1 was deposited on the hole injection layer to form a first hole transport layer having a thickness of 75 nm on the hole injection layer. Compound HT-3 was then introduced into another cell of the vacuum vapor deposition apparatus and was evaporated by applying an electric current to the cell, thereby forming a second hole transport layer having a thickness of 5 nm on the first hole transport layer. After forming the hole injection layer and the hole transport layers, a light-emitting layer was formed thereon as follows; Compound C-2 shown in Table 2 was introduced into a cell of the vacuum vapor depositing apparatus as a host, and compound C-3 was introduced into another cell as a dopant. The host material and the dopant material were evaporated at different rates, and the dopant was deposited in a doping amount of 2 wt % based on the total amount of the host and the dopant to form a light-emitting layer having a thickness of 20 nm on the second hole transport layer. Compound H1-211 was evaporated to form an electron buffer layer having a thickness of 5 nm on the light-emitting layer. Compound ETL-1 and compound EIL-1 were evaporated in a weight ratio of 4:6 to form an electron transport layer having a thickness of 30 nm on the electron buffer layer. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, an OLED was produced. All the materials used for producing the OLED were purified by vacuum sublimation at 10⁻⁶ torr.

The minimum time taken for luminance to decrease from 100% to 95% at a luminance of 1,770 nit (lifetime; T95) of the produced OLED was 51.6 hours.

### Device Example 5: Producing a Blue OLED Comprising the Electron Buffer Layer Compound According to the Present Disclosure

An ° LED was produced in the same manner as in Device Example 4, except that compound H1-212 was used as the electron buffer layer material.

The minimum time taken for luminance to decrease from 100% to 95% at a luminance of 1,770 nit (lifetime; T95) of the produced OLED was 54.5 hours.

### Device Example 6: Producing a Blue OLED Comprising the Electron Buffer Layer Compound According to the Present Disclosure

An OLED was produced in the same manner as in Device Example 4, except that compound H1-213 was used as the electron buffer layer material.

The minimum time taken for luminance to decrease from 100% to 95% at a luminance of 1,770 nit (lifetime; T95) of the produced OLED was 54.5 hours.

### Comparative Example 2: Producing a Blue OLED Comprising a Conventional Compound as an Electron Buffer Layer

An OLED was produced in the same manner as in Device Example 4, except that compound C-1 was used as the electron buffer layer material.

The minimum time taken for luminance to decrease from 100% to 95% at a luminance of 1,770 nit (lifetime; T95) of the produced OLED was 31.6 hours.

From Device Examples 4 to 6 and Comparative Example 2, it can be seen that the OLEDs using the organic electroluminescent compounds according to the present disclosure as electron buffer layer materials have an improved lifetime property. That is, the lifetime property of a blue organic electroluminescent device can be improved by comprising the compound of the present disclosure. Therefore, the blue organic electroluminescent device can also exhibit comparative performance, which can maintain a balance with the lifetime property of red and green organic electroluminescent devices, and thus it is expected to be applicable to various fields as well as displays.

The compounds used in the Device Examples and the Comparative Examples are shown in Table 2.

TABLE 2

| | |
|---|---|
| Hole Injection Layer/ Hole Transport Layer | 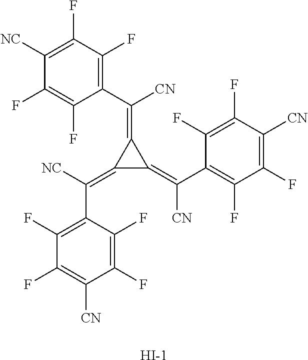 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 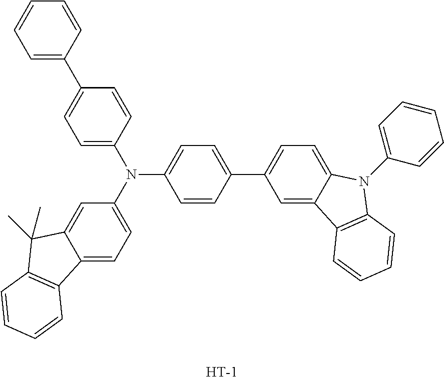 |
| | 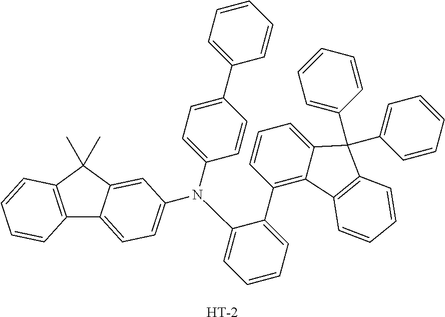 |
| | 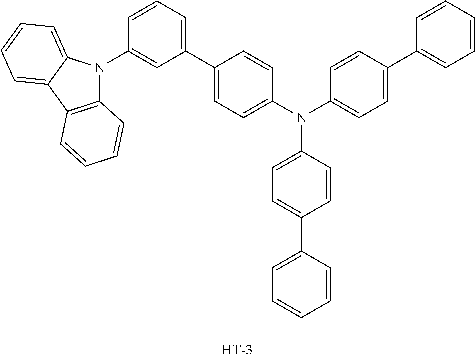 |
| Light- Emitting Layer/ Electron Buffer Layer | 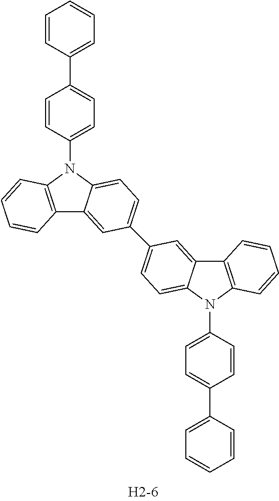 [SMILES: c1ccc(-c2ccc(-n3c4ccccc4c4cc(-c5ccc6c(c5)c5ccccc5n6-c5ccc(-c6ccccc6)cc5)ccc43)cc2)cc1] |
| | 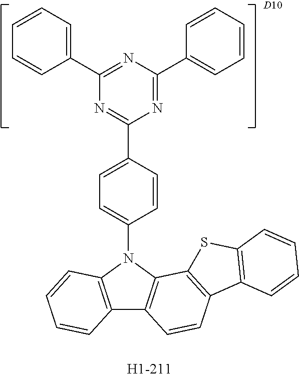 |
| | 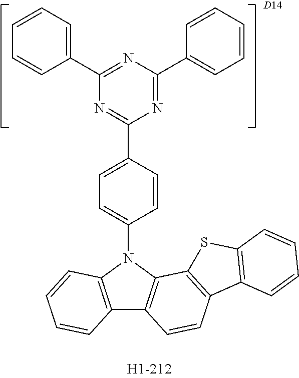 |
| | 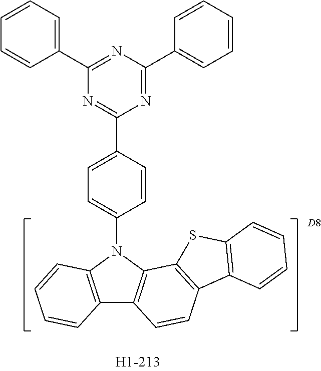 |
| | 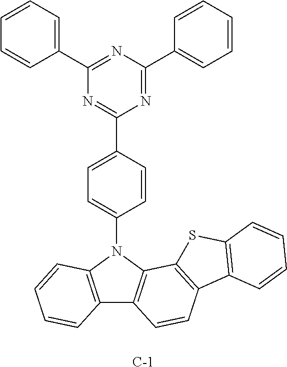 |
| | 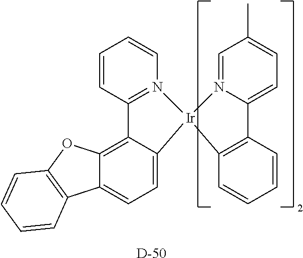 |
| | 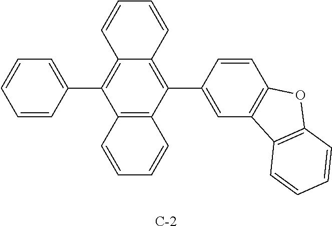 |
| | 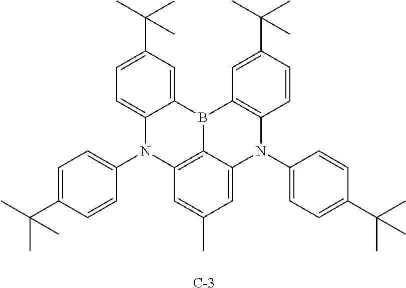 |
| Electron Transport Layer/ Electron Injection Layer | 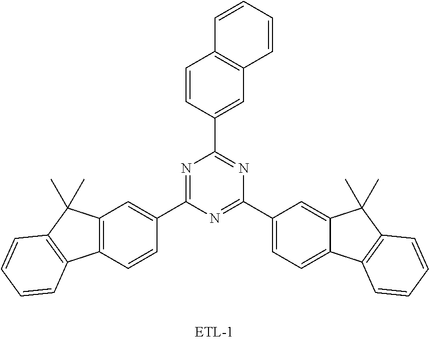 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 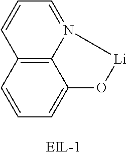 |

1. A plurality of host materials comprising a first host material comprising a compound represented by the following formula 1 and a second host material comprising a compound represented by the following formula 2, the compound represented by formula 1 and the compound represented by the following formula 2 being different from each other: 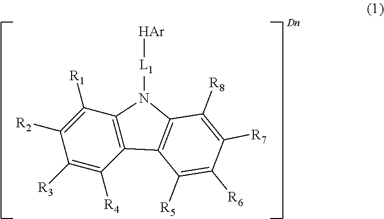
in formula 1,
HAr represents a substituted or unsubstituted (3- to 30-membered)heteroaryl;
L₁ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene,
R₁ to R₅, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NR₁₃R₁₄ or —SiR₁₅R₁₆R₁₇; or may be linked to an adjacent substituent to form a ring(s),
with the proviso that at least one group of group R₅ and R₆, group R₆ and R₇, and group R₇ and R₅ of formula 1 are fused with * of the following formula 1-a to form a ring(s), 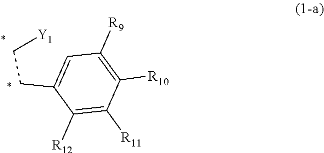
in formula 1-a,
Y₁ represents O or S,
R₉ to R₁₂, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NR₁₈R₁₉ or —SiR₂₀R₂₁R₂₂,
R₁₃ to R₂₂, each independently, represent hydrogen, deuterium, a halogen, a cyano a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl; or may be linked to an adjacent substituent to form a ring(s);
Dₙ represents that n hydrogens are replaced with deuterium; and
n represents an integer of 1 to 50; 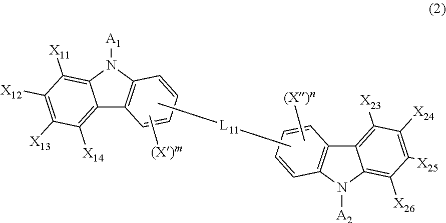
in formula 2,
A₁ and A₂, each independently, represent a substituted or unsubstituted (C6-C30)aryl, or a substituted or unsubstituted (3- to 30-membered)heteroaryl;
L₁₁ represents a single bond, or a substituted or unsubstituted (C6-C30)arylene;
X′, X″, X₁₁ to X₁₄, and X₂₃ to X₂₆, each independently, represent hydrogen, deuterium, a halogen, a cyano a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C30)aryl, a substituted or unsubstituted (3- to 30-membered)heteroaryl, —NR₂₃R₂₄ or —SiR₂₅R₂₆R₂₇; or may be linked to an adjacent substituent to form a ring(s);
R₂₃ to R₂₇, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl; or may be linked to an adjacent substituent to form a ring(s);
m and n, each independently, represent an integer of 1 to 3; and
if m and n are an integer of 2 or more, each of X′ and each of X″ may be the same or different.

2. The plurality of host materials according to claim 1, wherein the formula 1 is represented by at least one of the following formulas 1-1 to 1-6: 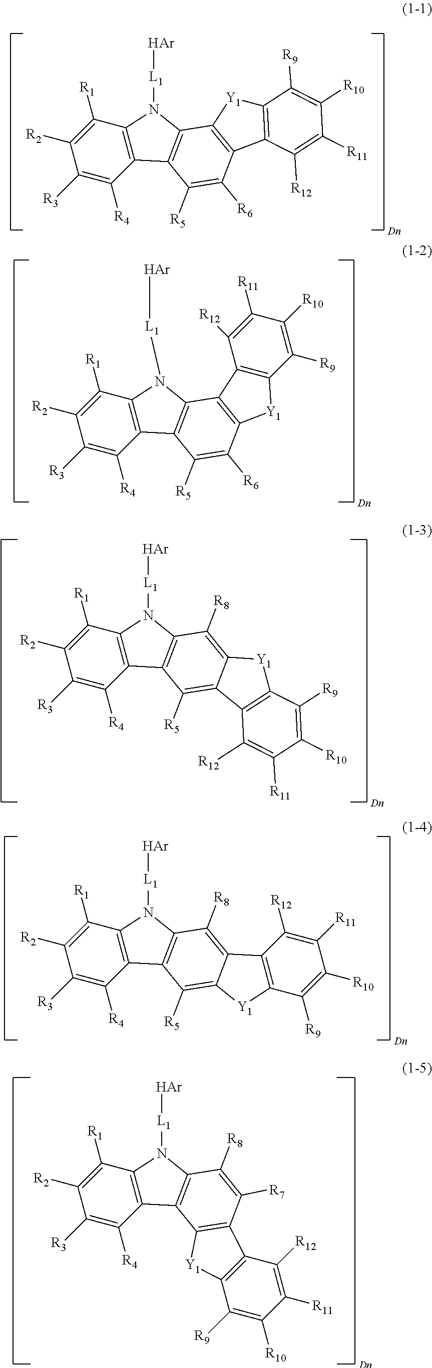 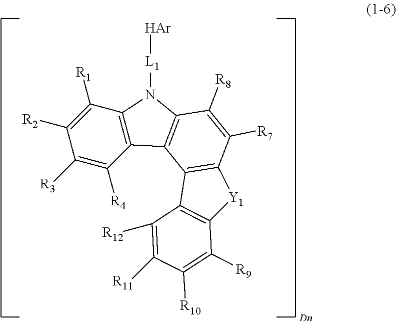
in formulas 1-1 to 1-6,
R₁ to R₁₂, Y₁, L₁, HAr and Dn are as defined in claim 1.

3. The plurality of host materials according to claim 1, wherein HAr of formula 1 is a substituted or unsubstituted triazinyl, a substituted or unsubstituted pyridyl, a substituted or unsubstituted pyrimidinyl, a substituted or unsubstituted quinazolinyl, a substituted or unsubstituted benzoquinazolinyl, a substituted or unsubstituted quinoxalinyl, a substituted or unsubstituted benzoquinoxalinyl, a substituted or unsubstituted quinolyl, a substituted or unsubstituted benzoquinolyl, a substituted or unsubstituted isoguinolyl, a substituted or unsubstituted benzoisoquinolyl, a substituted or unsubstituted triazolyl, a substituted or unsubstituted pyrazolyl, a substituted or unsubstituted naphthyridinyl, a substituted or unsubstituted triazanaphthyl, or a substituted or unsubstituted benzothienopyrimidinyl.

4. The plurality of host materials according to claim 1, wherein L₁ of formula 1 is a single bond or a dibenzofuranylene unsubstituted or substituted with at least one deuterium, or is represented by any one selected from the group consisting of the following: 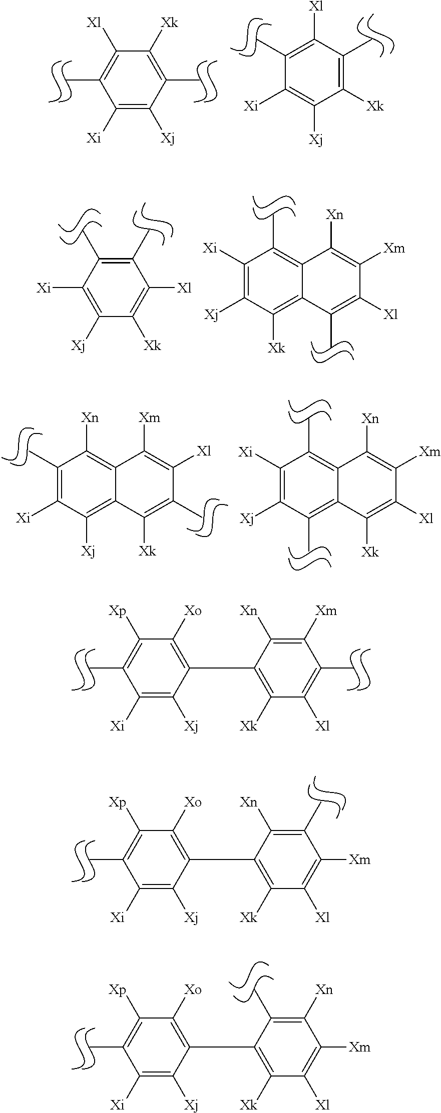 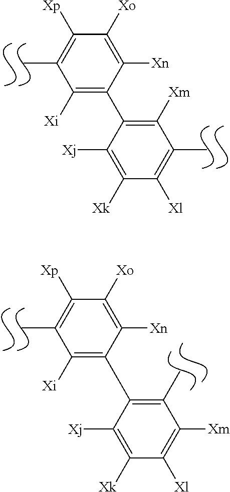
in the formulas above,
Xᵢ to Xₚ, each independently, represent hydrogen, deuterium, a halogen, a cyano, a substituted or unsubstituted (C1-C30)alkyl, a substituted or unsubstituted (C2-C30)alkenyl, a substituted or unsubstituted (C2-C30)alkynyl, a substituted or unsubstituted (C3-C30)cycloalkyl, a substituted or unsubstituted (C6-C30)aryl, a substituted or unsubstituted (3- to 30-membered)heteroaryl, —NR₂₈R₂₉ or —SiR₃₀R₃₁R₃₂; or may be linked to an adjacent substituent to form a ring(s); and
R₂₈ to R₃₂, each independently; represent hydrogen; deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl; a substituted or unsubstituted (3- to 7-membered)heterocycloalkyl, a substituted or unsubstituted (C6-C30)aryl, or a substituted or unsubstituted (3- to 30-membered)heteroaryl; or may be linked to an adjacent substituent to form a ring(s).

5. The plurality of host materials according to claim 1; wherein the formula 2 is represented by at least one of the following formulas 2-1 to 2-8: 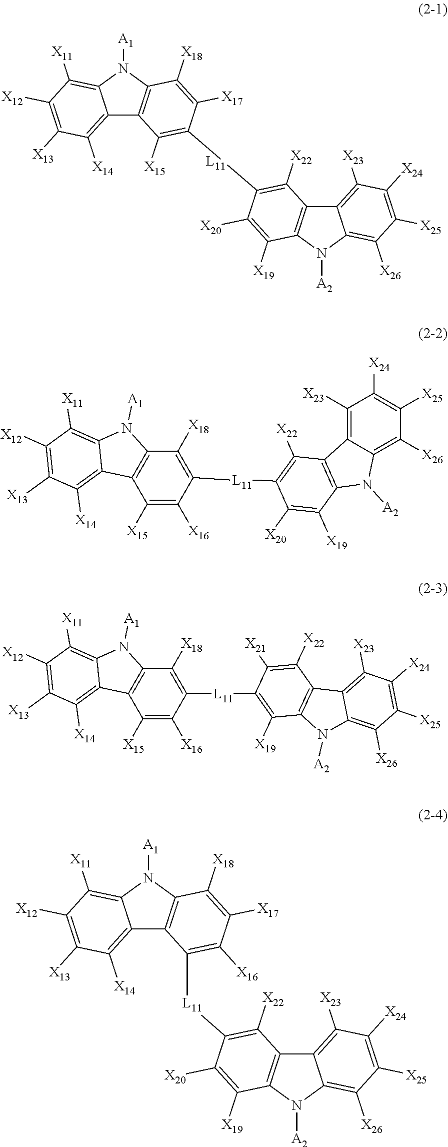 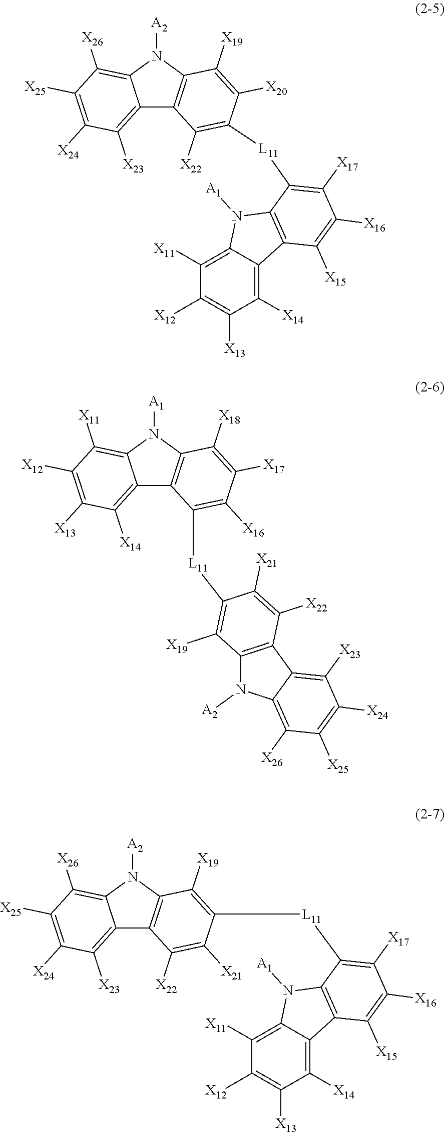 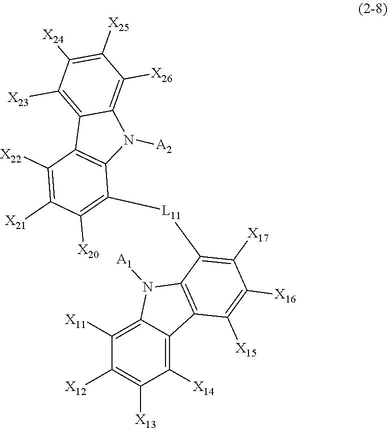
in formulas 2-1 to 2-8,
A₁, A₂, X₁₁ to X₁₄, and X₂₃ to X₂₆ are as defined in claim 1; and
X₁₅ to X₂₂, each independently, are the same as the definition of X′ in claim 1.

6. The plurality of host materials according to claim 1, wherein A₁ and A₂ of formula 2, each independently, is a substituted or unsubstituted phenyl, a substituted or unsubstituted biphenyl, a substituted or unsubstituted terphenyl, a substituted or unsubstituted naphthyl, a substituted or unsubstituted fluorenyl, a substituted or unsubstituted benzofluorenyl, a substituted or unsubstituted triphenylenyl, a substituted or unsubstituted fluoranthenyl, a substituted or unsubstituted phenanthrenyl, a substituted or unsubstituted dibenzofuranyl, a substituted or unsubstituted carbazolyl, or a substituted or unsubstituted dibenzothiophenyl.

7. The plurality of host materials according to claim 1, wherein the substituent(s) of the substituted alkyl(ene), the substituted alkenyl, the substituted alkynyl, the substituted aryl(ene), the substituted heteroaryl(ene), the substituted cycloalkyl(ene), the substituted cycloalkenyl; and the substituted heterocycloalkyl, each independently, are at least one selected from the group consisting of deuterium; a halogen; a cyano; a carboxyl; a nitro; a hydroxyl; a phosphineoxide; a (C1-C30)alkyl; a halo(C1-C30)alkyl; a (C2-C30)alkenyl; a (C2-C30)alkynyl: a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of deuterium and a (C6-C30)aryl(s); a (C6-C30)aryl unsubstituted or substituted with at least one of deuterium and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C2-C30)alkenylamino; a mono- or di-(C6-C30)arylamino unsubstituted or substituted with a (C1-C30)alkyl(s); a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (01-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

8. The plurality of host materials according to claim 1, wherein the compound represented by formula 1 is at least one selected from the following compounds:

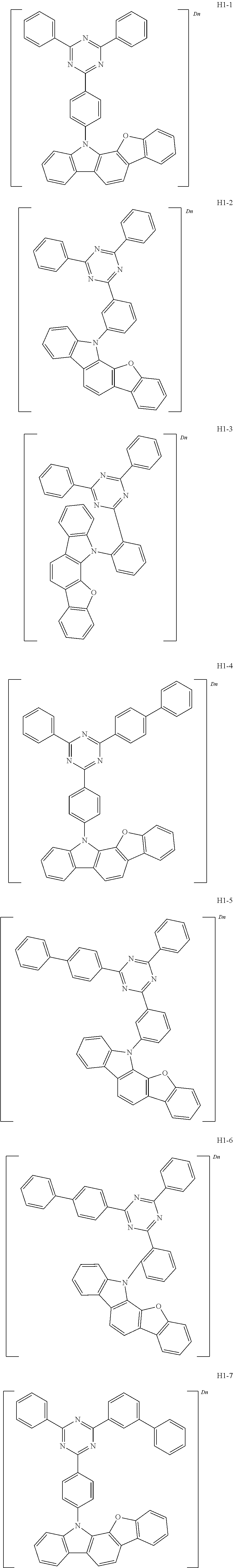

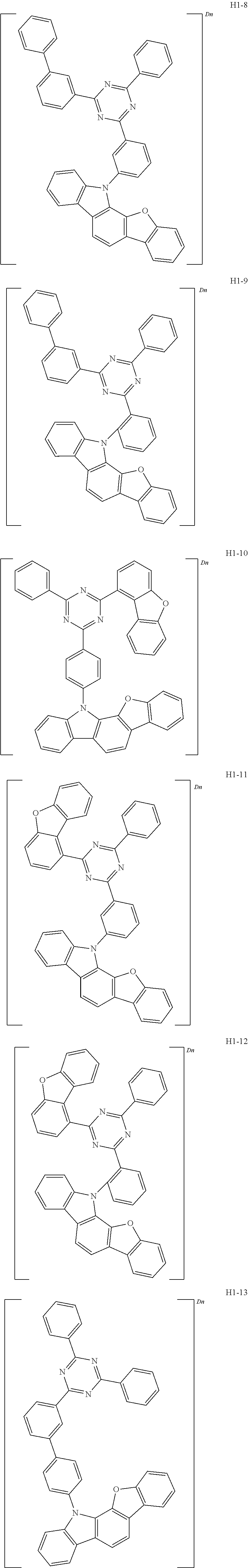

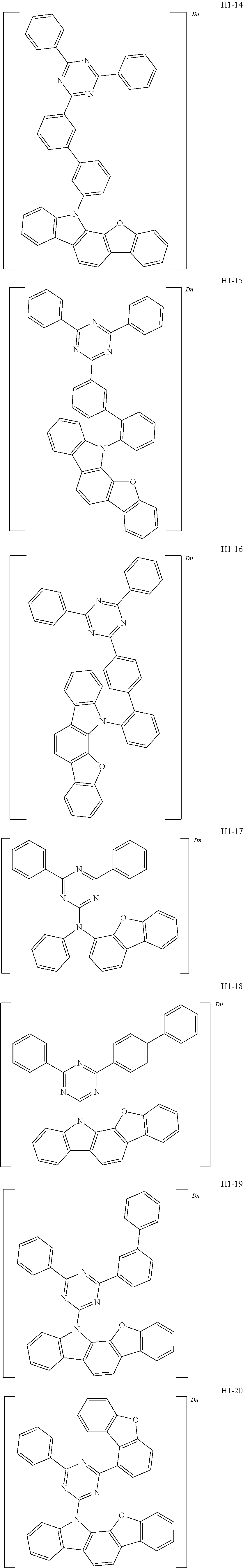

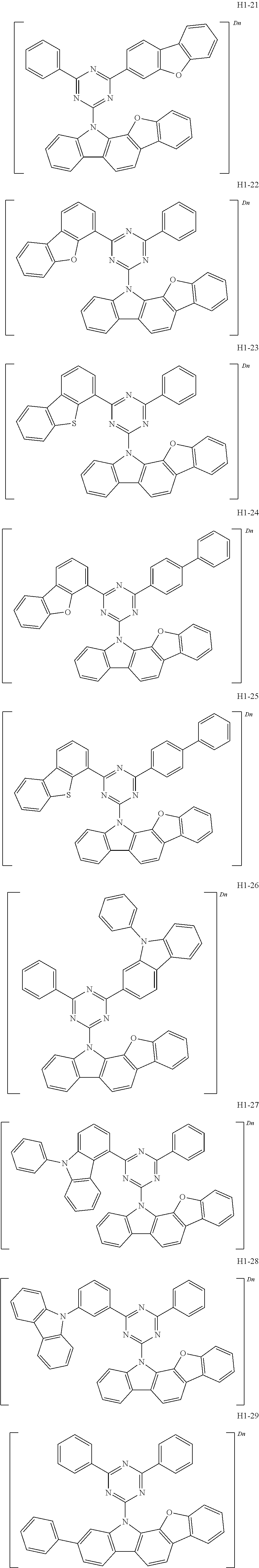

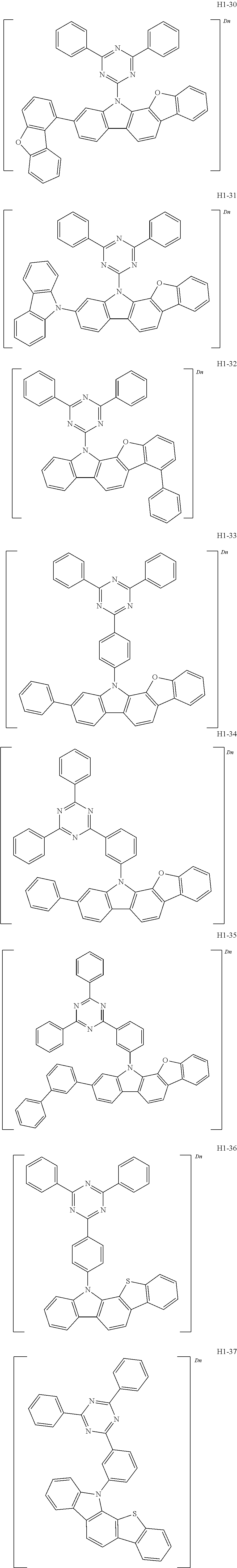

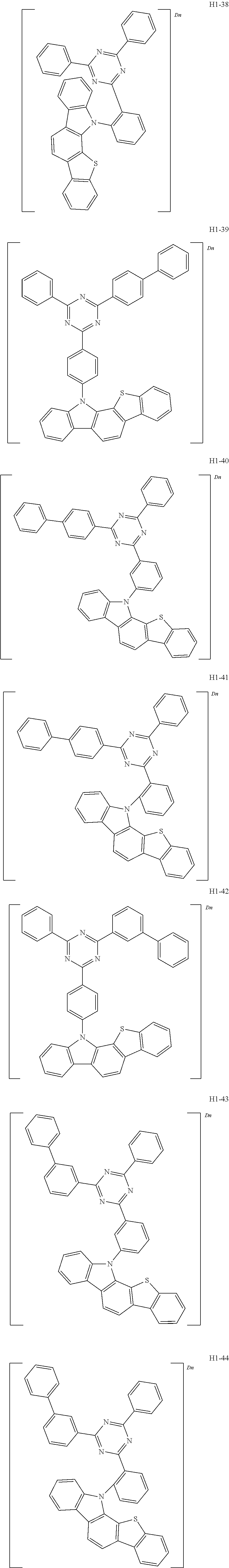

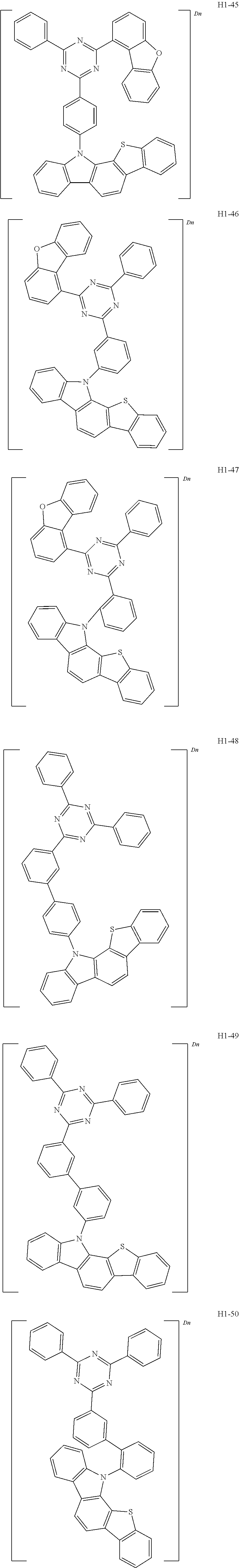

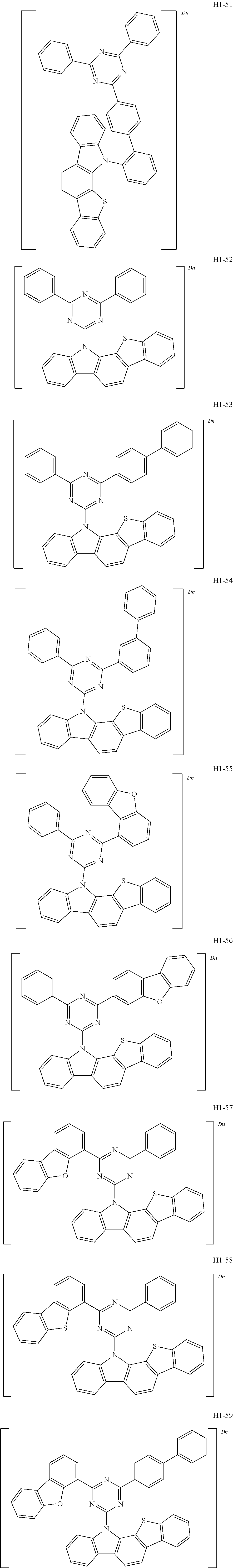

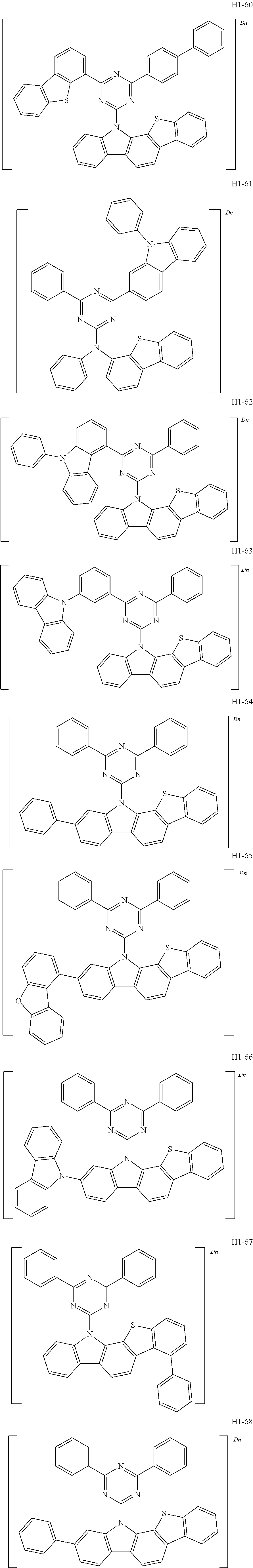

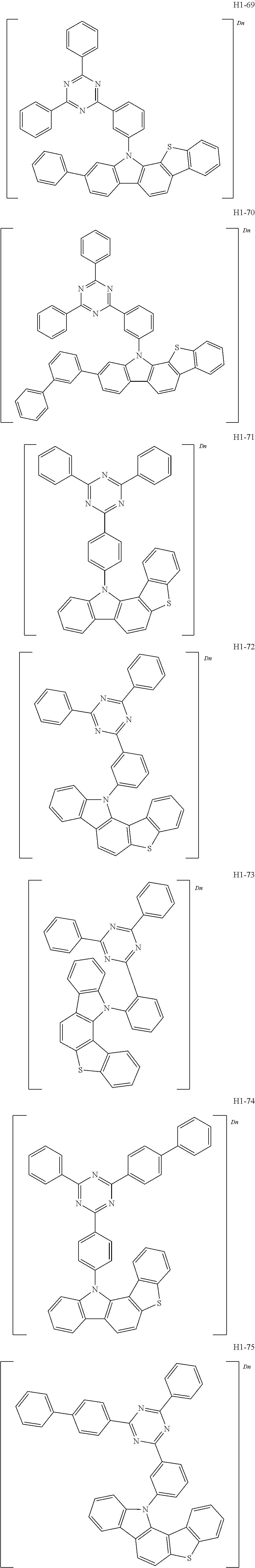

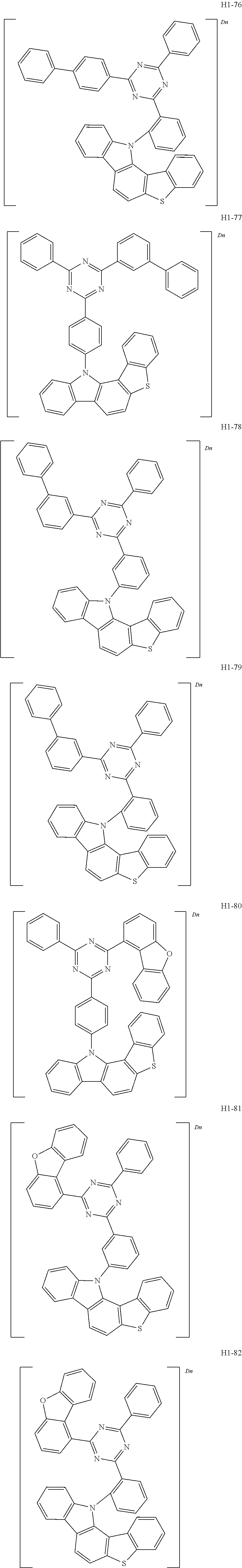

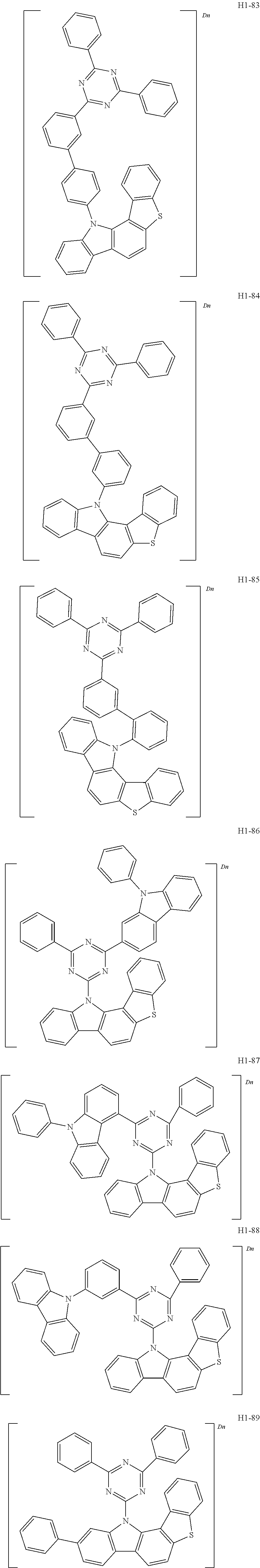

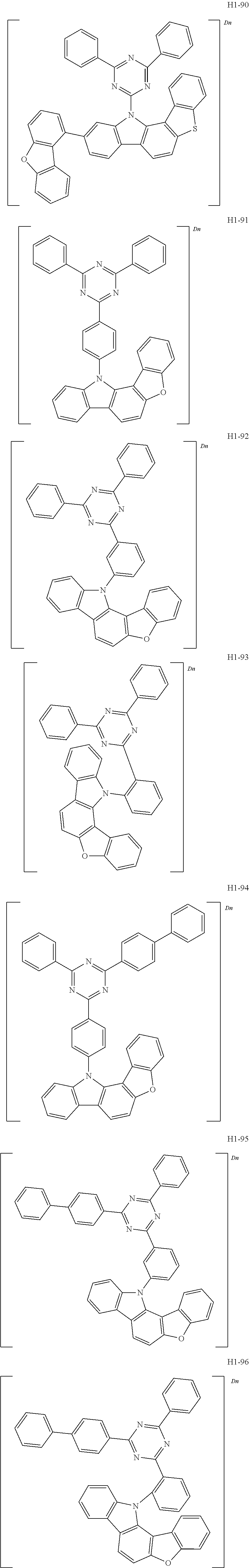

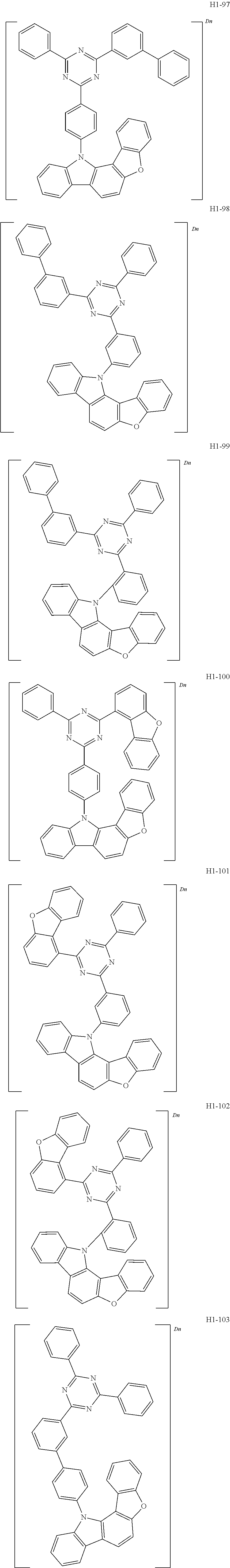

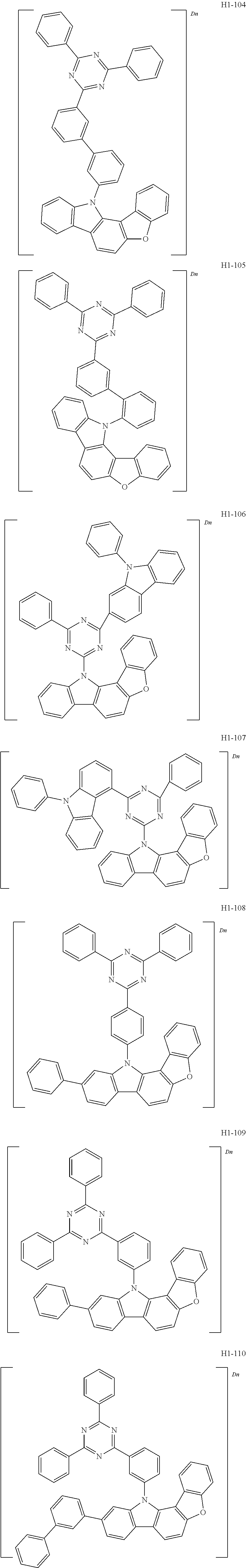

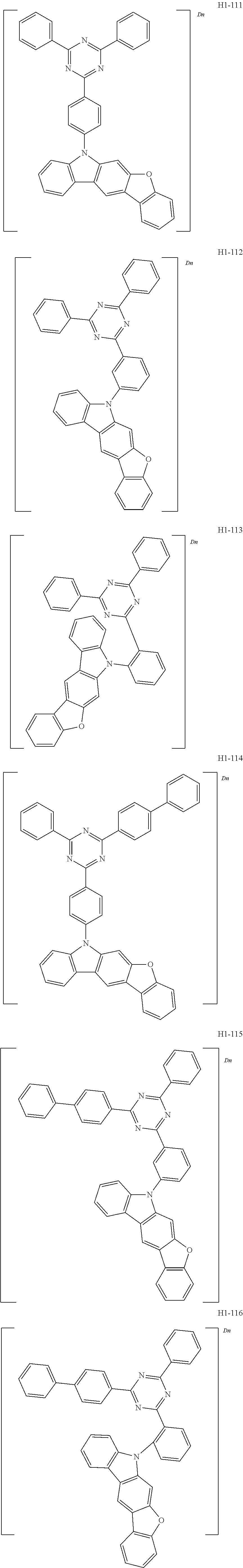

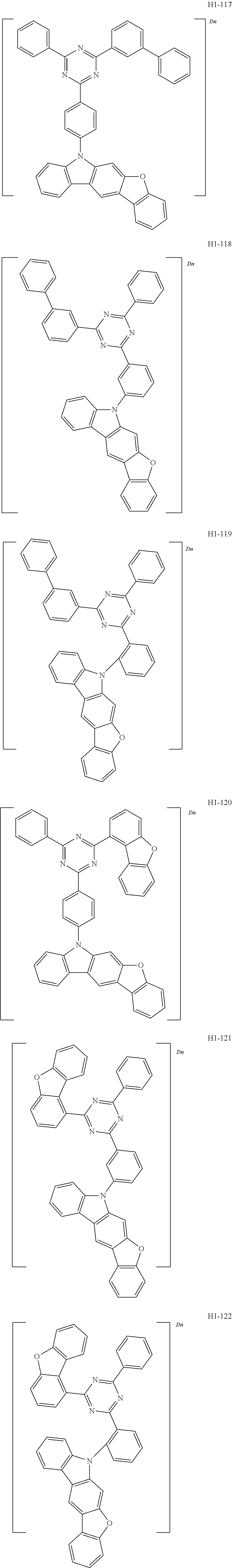

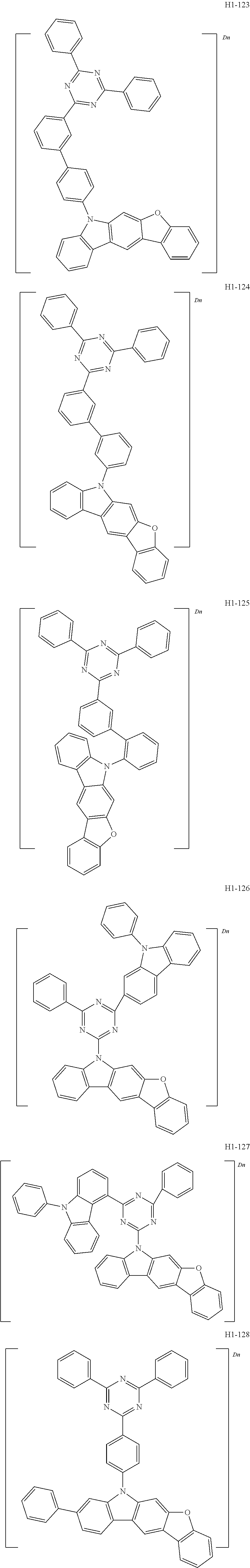

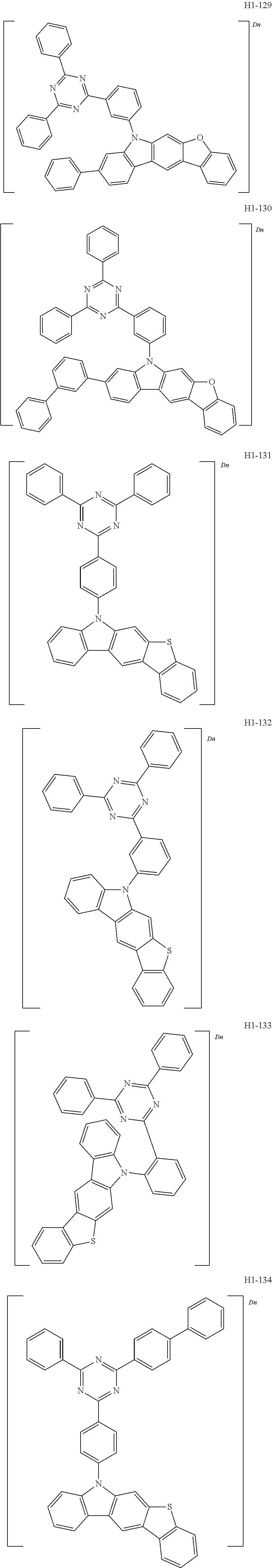

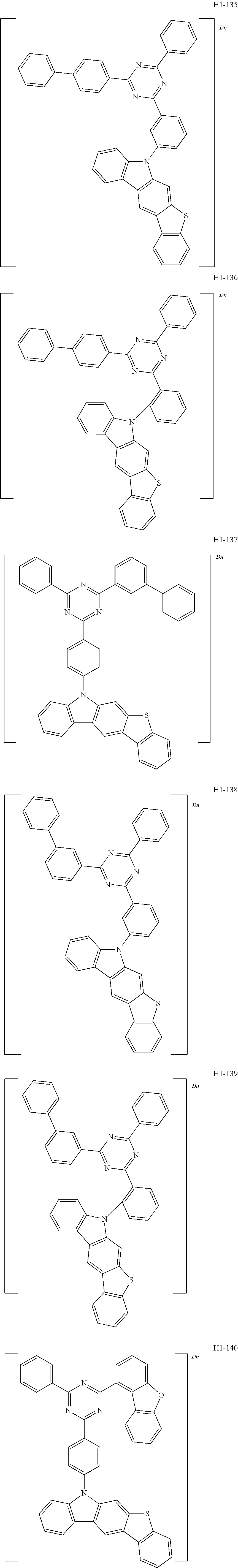

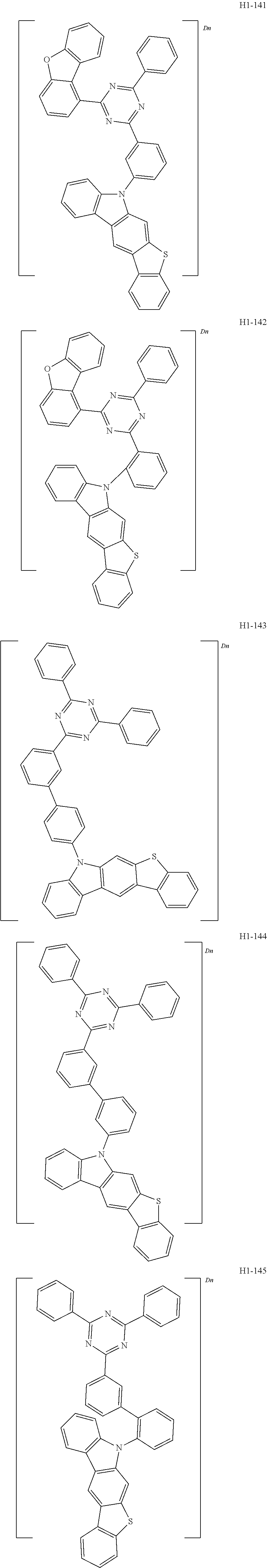

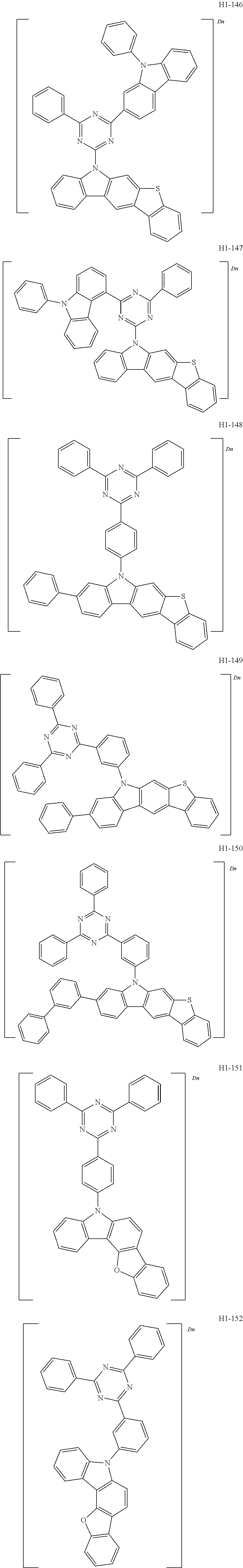

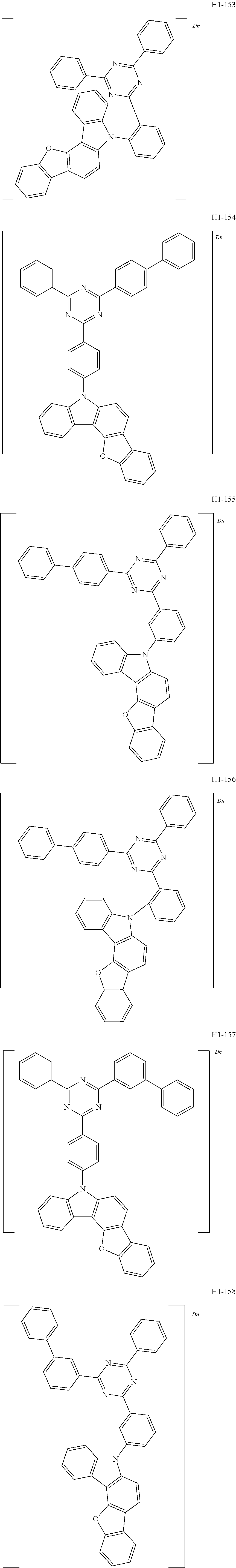

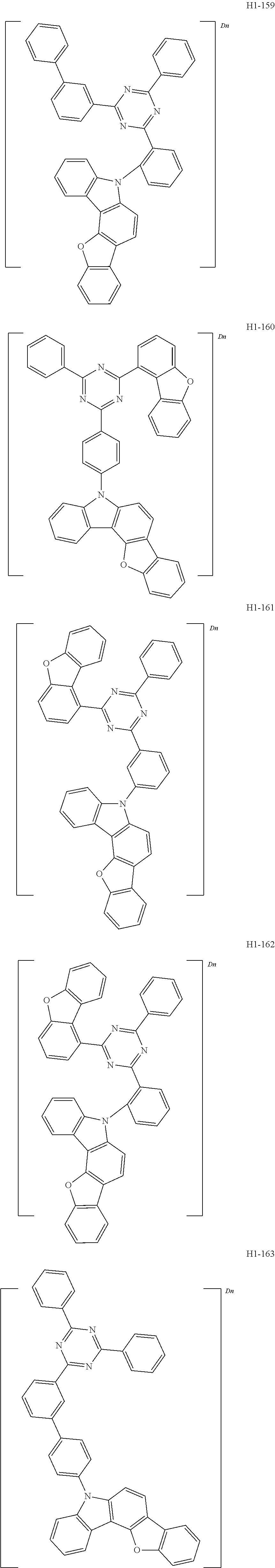

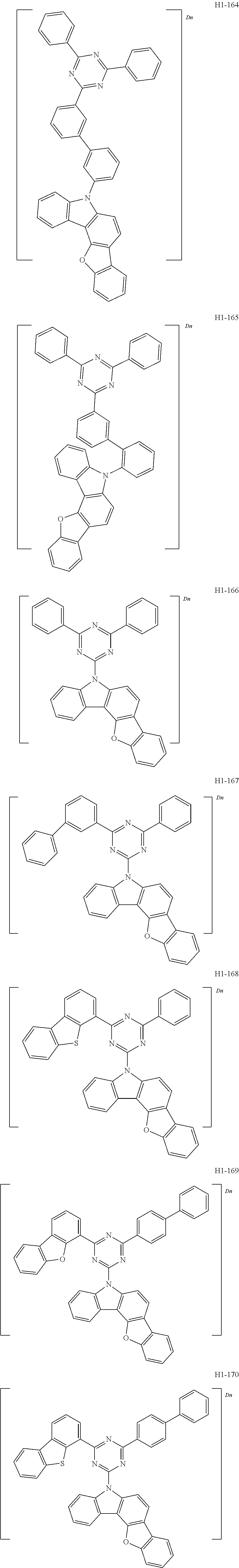

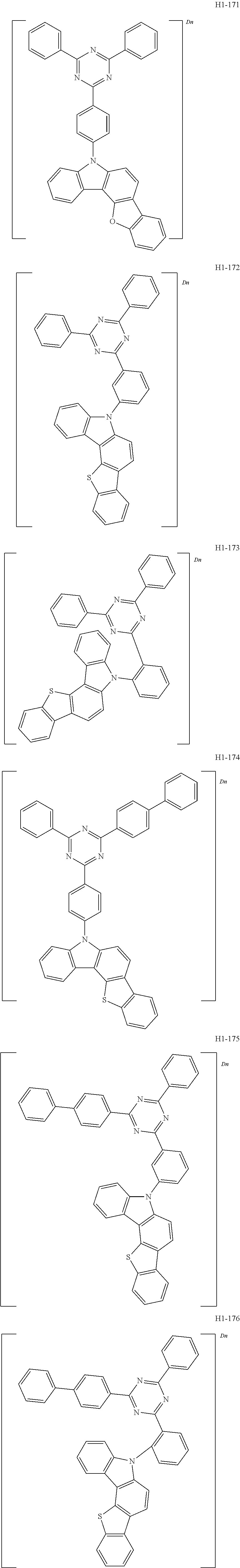

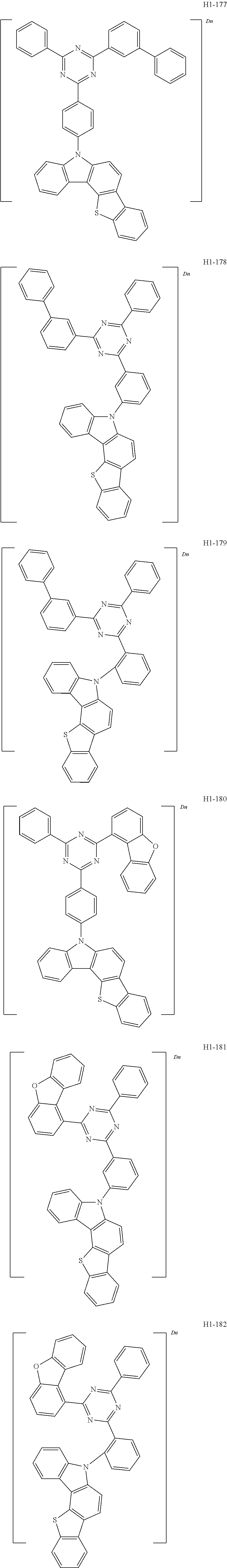

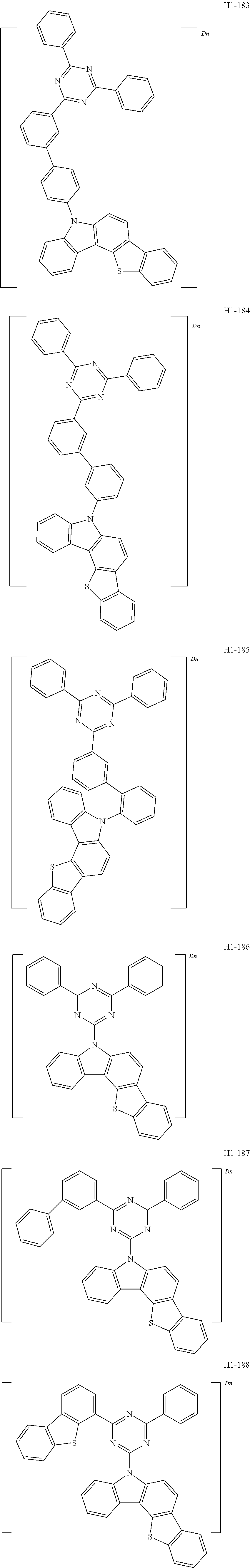

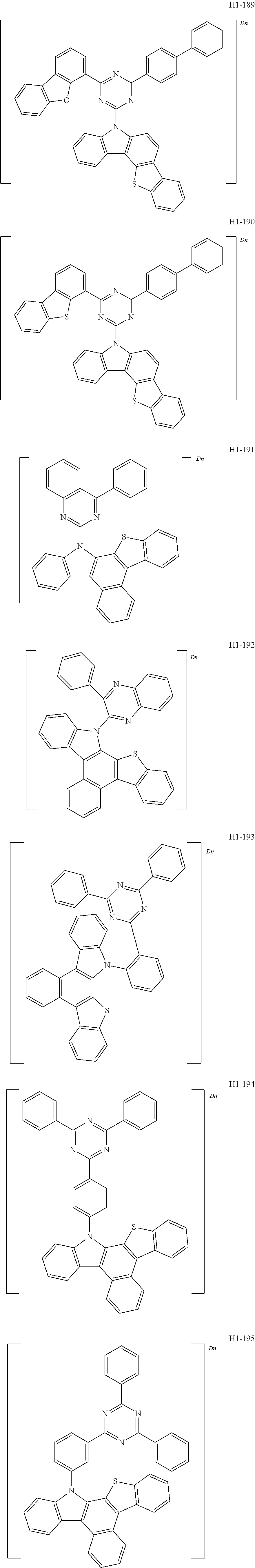

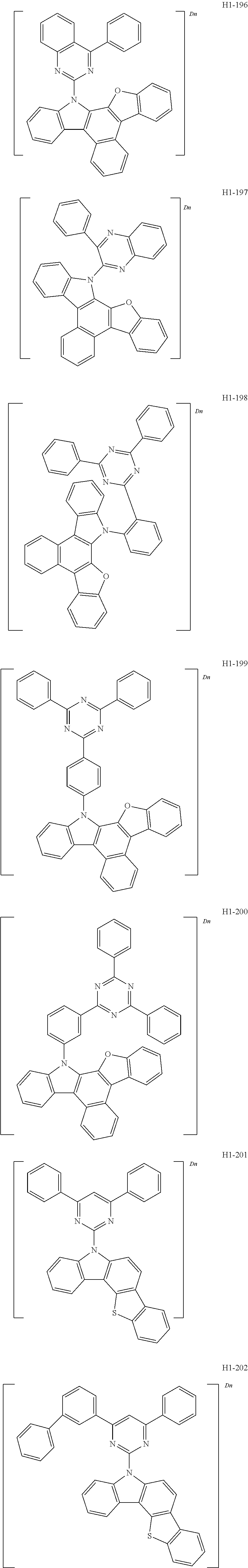

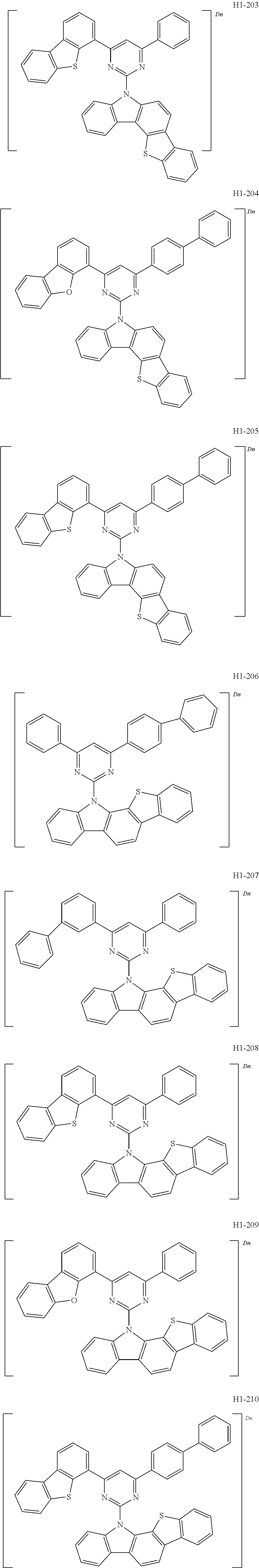

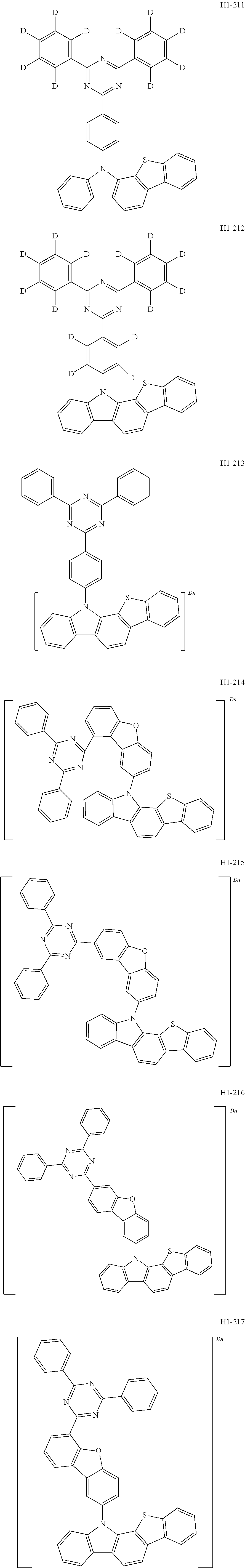

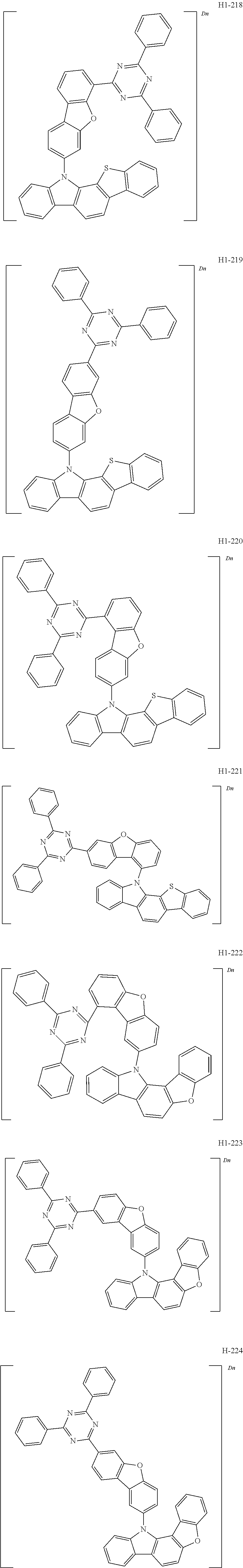

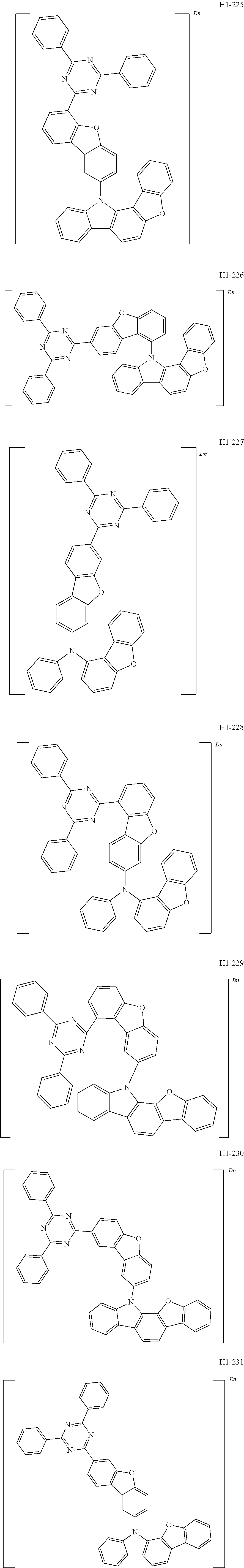

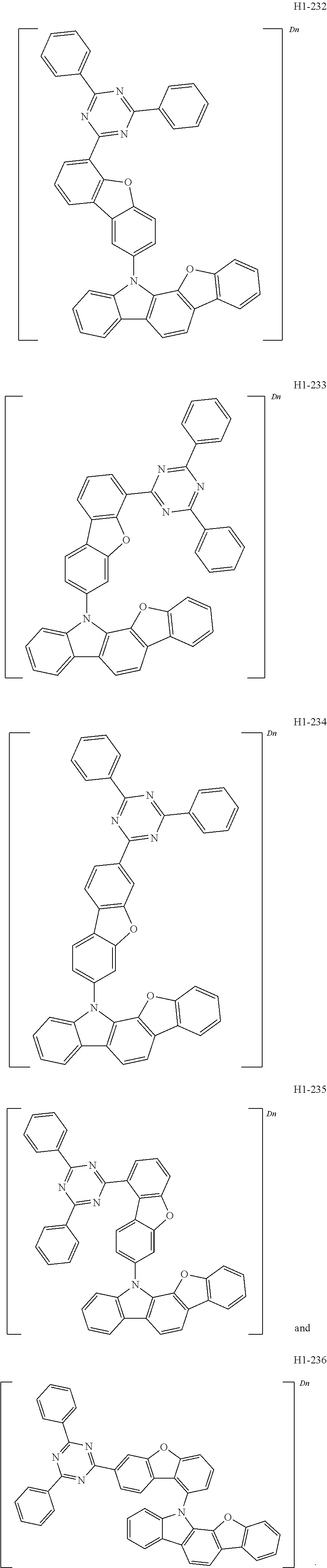

9. The plurality of host materials according to claim 1, wherein the compound represented by formula 2 is at least one selected from the following compounds:

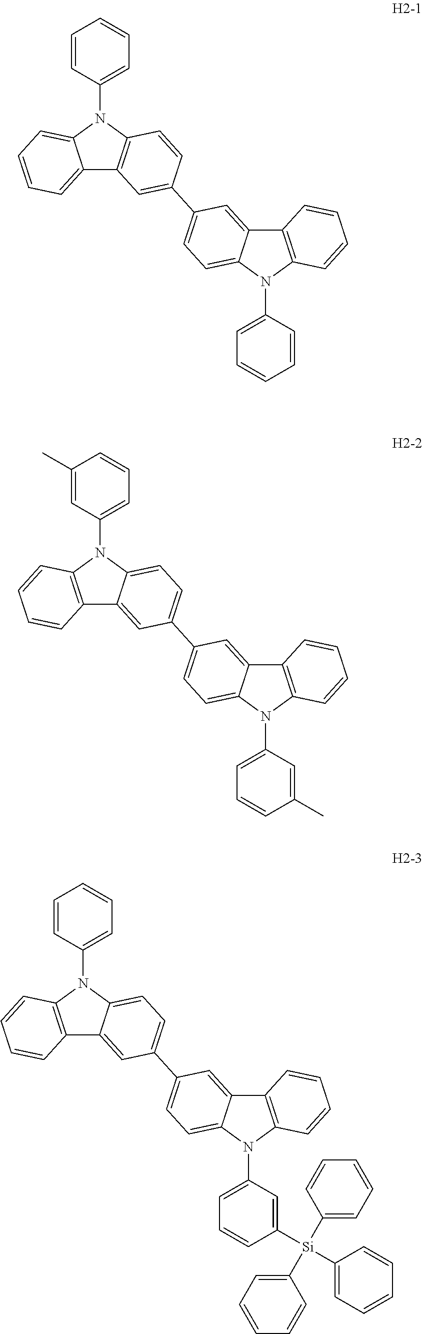 [SMILES: c1ccc(-n2c3ccccc3c3cc(-c4ccc5c(c4)c4ccccc4n5-c4ccccc4)ccc32)cc1 ; Cc1cccc(-n2c3ccccc3c3cc(-c4ccc5c(c4)c4ccccc4n5-c4cccc(C)c4)ccc32)c1 ; c1ccc(-n2c3ccccc3c3cc(-c4ccc5c(c4)c4ccccc4n5-c4cccc([Si](c5ccccc5)(c5ccccc5)c5ccccc5)c4)ccc32)cc1]

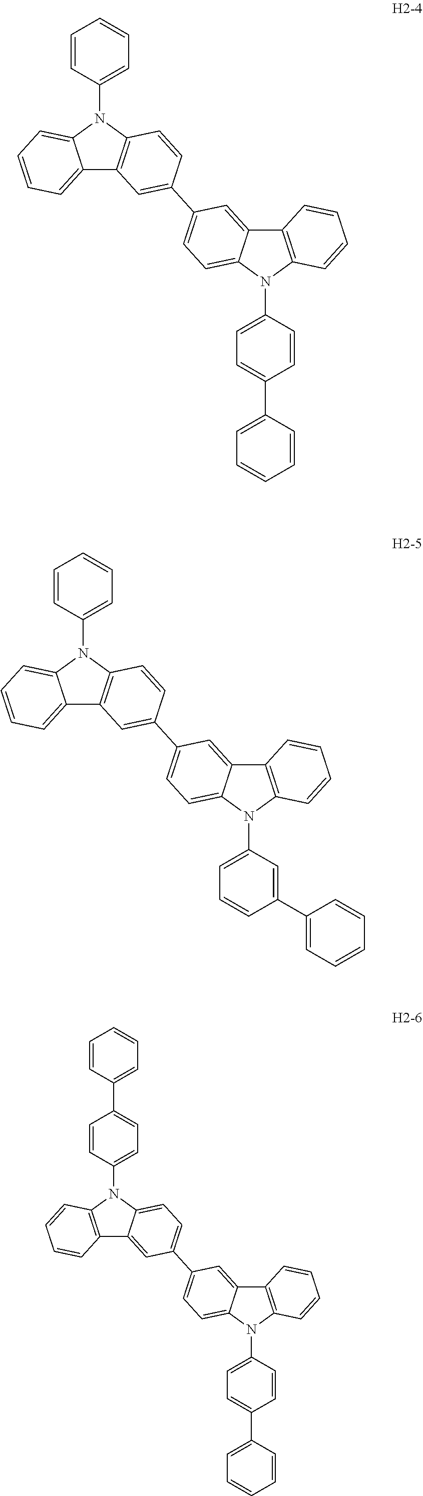 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

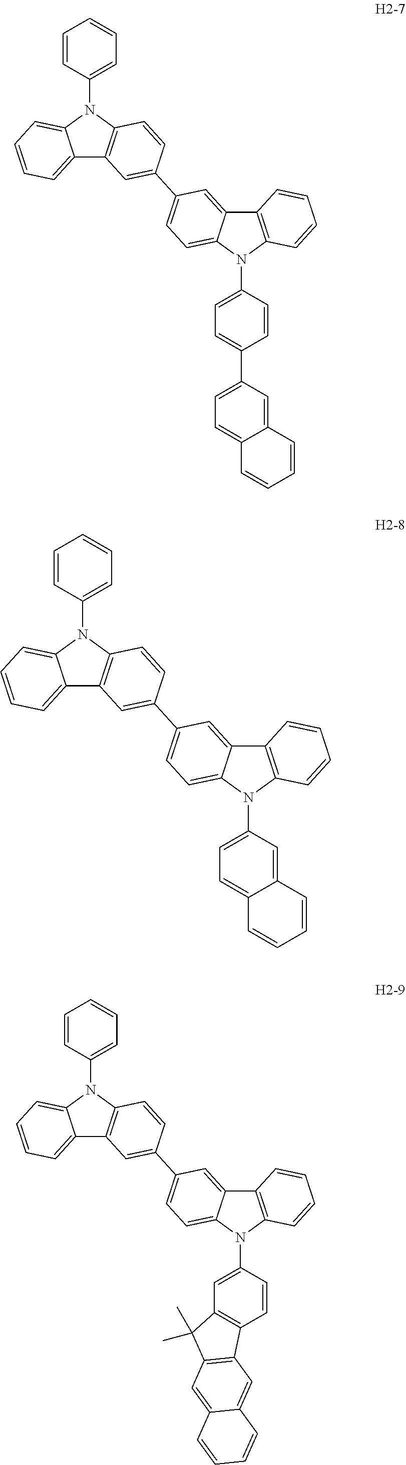 [SMILES: c1ccc(-n2c3ccccc3c3cc(-c4ccc5c(c4)c4ccccc4n5-c4ccc(-c5ccc6ccccc6c5)cc4)ccc32)cc1 ; c1ccc(-n2c3ccccc3c3cc(-c4ccc5c(c4)c4ccccc4n5-c4ccc5ccccc5c4)ccc32)cc1 ; CC1(C)c2cc(-n3c4ccccc4c4cc(-c5ccc6c(c5)c5ccccc5n6-c5ccccc5)ccc43)ccc2-c2cc3ccccc3cc21]

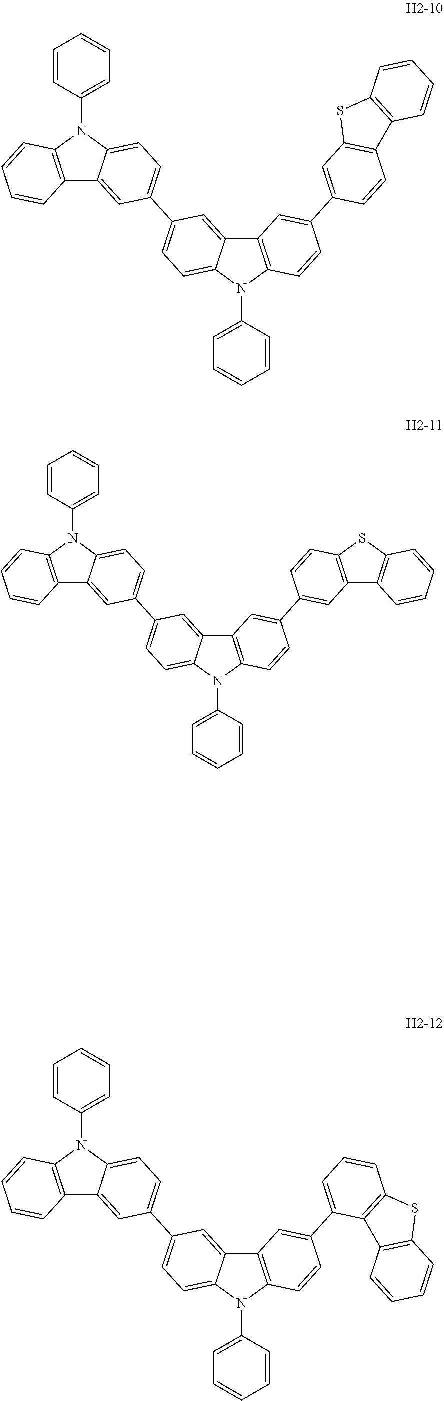 [SMILES: c1ccc(-n2c3ccccc3c3cc(-c4ccc5c(c4)c4cc(-c6ccc7c(c6)sc6ccccc67)ccc4n5-c4ccccc4)ccc32)cc1 ; c1ccc(-n2c3ccccc3c3cc(-c4ccc5c(c4)c4cc(-c6ccc7sc8ccccc8c7c6)ccc4n5-c4ccccc4)ccc32)cc1 ; c1ccc(-n2c3ccccc3c3cc(-c4ccc5c(c4)c4cc(-c6cccc7sc8ccccc8c67)ccc4n5-c4ccccc4)ccc32)cc1]

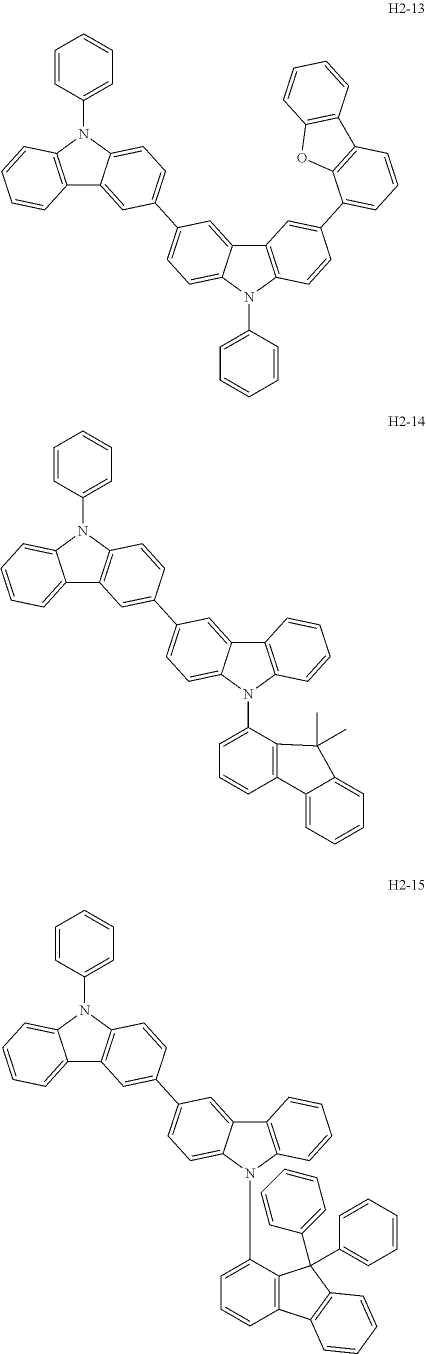 [SMILES: c1ccc(-n2c3ccccc3c3cc(-c4ccc5c(c4)c4cc(-c6cccc7c6oc6ccccc67)ccc4n5-c4ccccc4)ccc32)cc1 ; CC1(C)c2ccccc2-c2cccc(-n3c4ccccc4c4cc(-c5ccc6c(c5)c5ccccc5n6-c5ccccc5)ccc43)c21 ; c1ccc(-n2c3ccccc3c3cc(-c4ccc5c(c4)c4ccccc4n5-c4cccc5c4C(c4ccccc4)(c4ccccc4)c4ccccc4-5)ccc32)cc1]

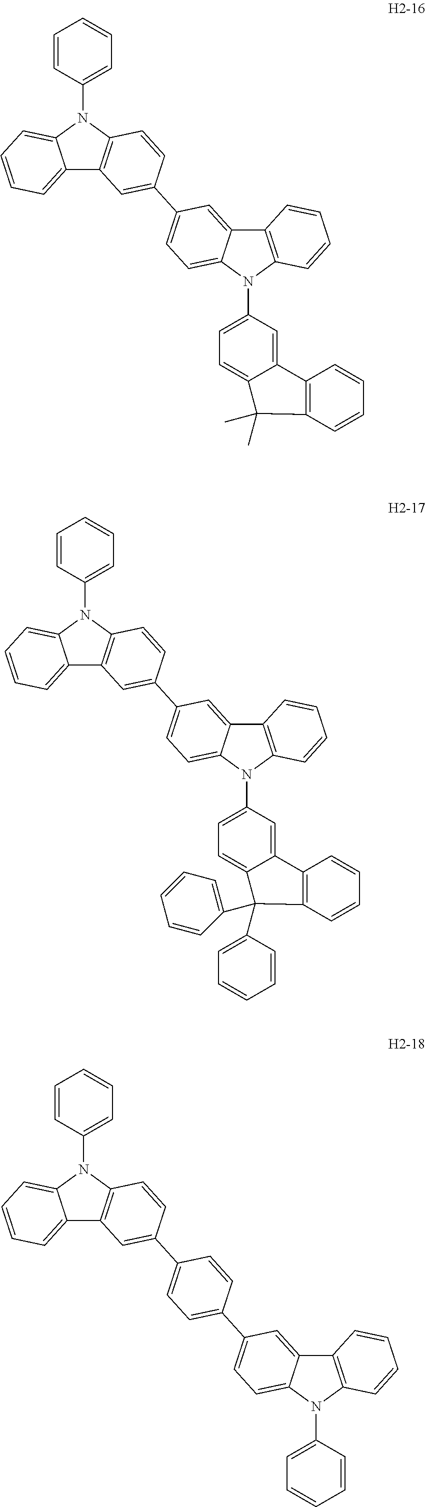 [SMILES: CC1(C)c2ccccc2-c2cc(-n3c4ccccc4c4cc(-c5ccc6c(c5)c5ccccc5n6-c5ccccc5)ccc43)ccc21 ; c1ccc(-n2c3ccccc3c3cc(-c4ccc5c(c4)c4ccccc4n5-c4ccc5c(c4)-c4ccccc4C5(c4ccccc4)c4ccccc4)ccc32)cc1 ; c1ccc(-n2c3ccccc3c3cc(-c4ccc(-c5ccc6c(c5)c5ccccc5n6-c5ccccc5)cc4)ccc32)cc1]

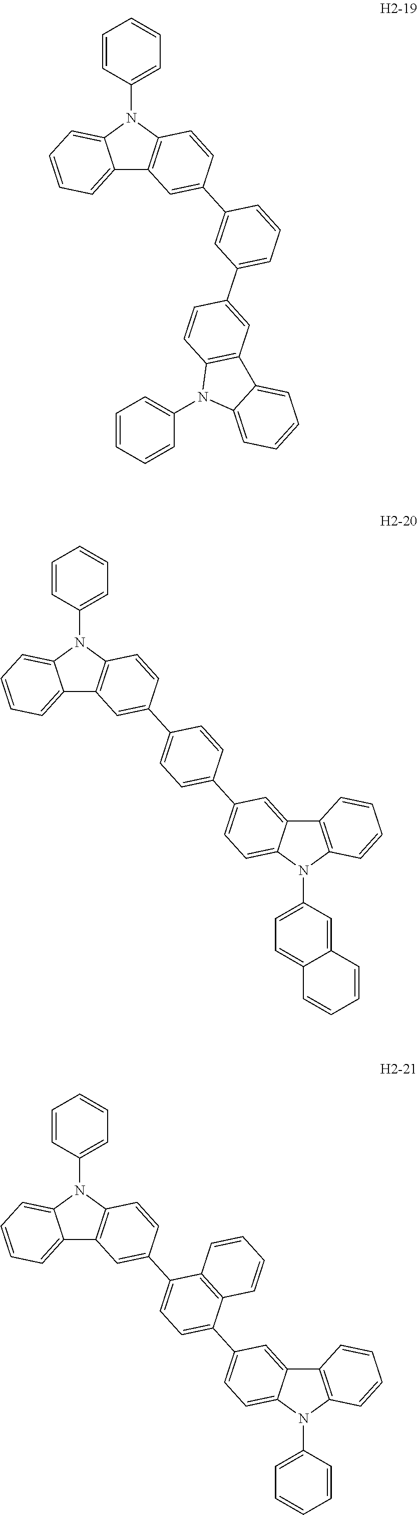 [SMILES: c1ccc(-n2c3ccccc3c3cc(-c4cccc(-c5ccc6c(c5)c5ccccc5n6-c5ccccc5)c4)ccc32)cc1 ; c1ccc(-n2c3ccccc3c3cc(-c4ccc(-c5ccc6c(c5)c5ccccc5n6-c5ccc6ccccc6c5)cc4)ccc32)cc1 ; c1ccc(-n2c3ccccc3c3cc(-c4ccc(-c5ccc6c(c5)c5ccccc5n6-c5ccccc5)c5ccccc45)ccc32)cc1]

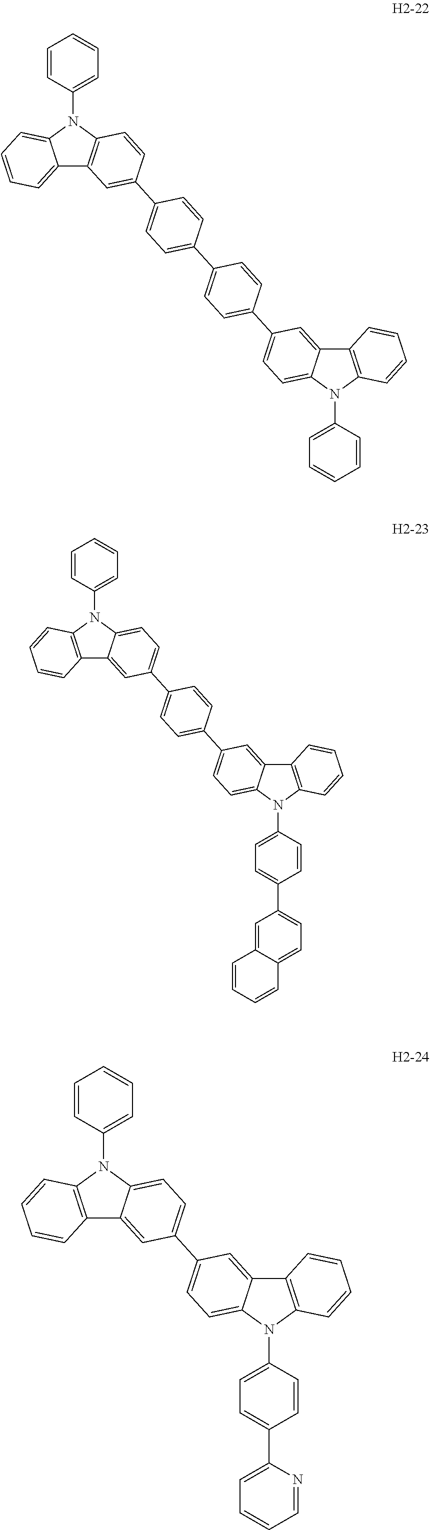 [SMILES: c1ccc(-n2c3ccccc3c3cc(-c4ccc(-c5ccc(-c6ccc7c(c6)c6ccccc6n7-c6ccccc6)cc5)cc4)ccc32)cc1 ; c1ccc(-n2c3ccccc3c3cc(-c4ccc(-c5ccc6c(c5)c5ccccc5n6-c5ccc(-c6ccc7ccccc7c6)cc5)cc4)ccc32)cc1 ; c1ccc(-n2c3ccccc3c3cc(-c4ccc5c(c4)c4ccccc4n5-c4ccc(-c5ccccn5)cc4)ccc32)cc1]

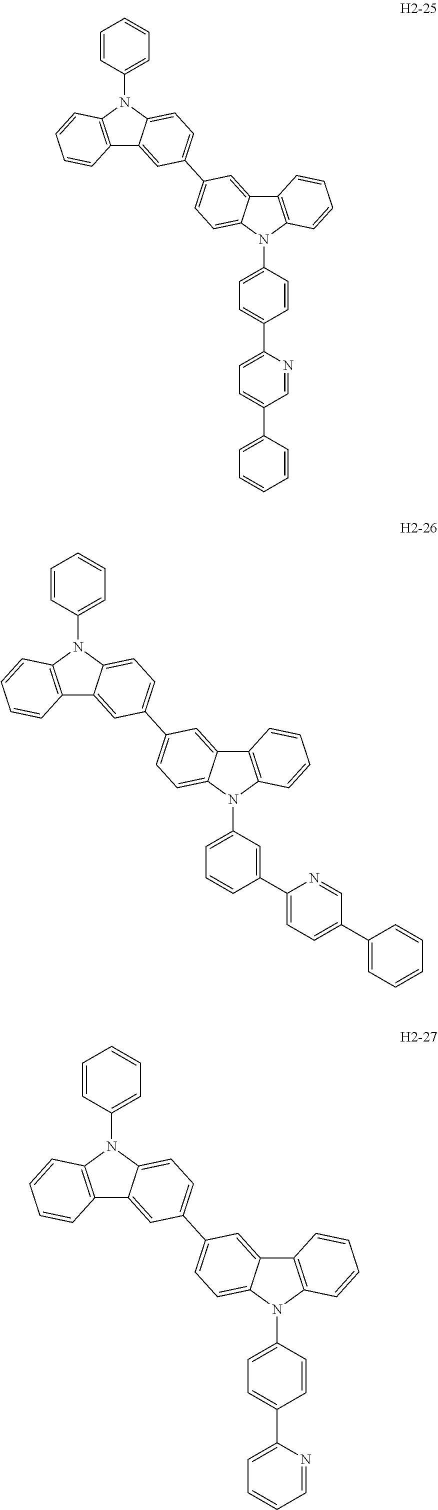 [SMILES: c1ccc(-c2ccc(-c3ccc(-n4c5ccccc5c5cc(-c6ccc7c(c6)c6ccccc6n7-c6ccccc6)ccc54)cc3)nc2)cc1 ; c1ccc(-c2ccc(-c3cccc(-n4c5ccccc5c5cc(-c6ccc7c(c6)c6ccccc6n7-c6ccccc6)ccc54)c3)nc2)cc1 ; c1ccc(-n2c3ccccc3c3cc(-c4ccc5c(c4)c4ccccc4n5-c4ccc(-c5ccccn5)cc4)ccc32)cc1]

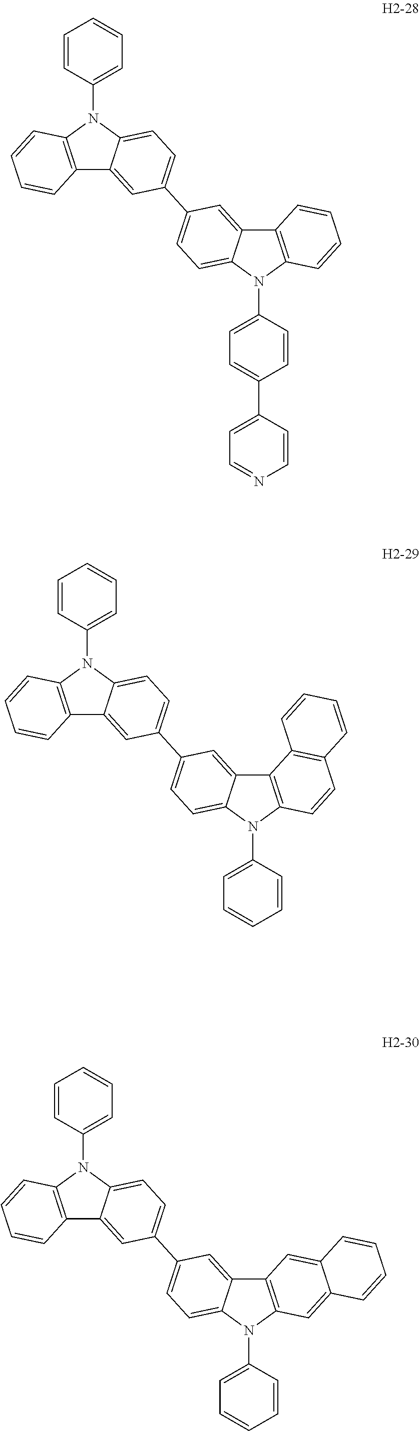 [SMILES: c1ccc(-n2c3ccccc3c3cc(-c4ccc5c(c4)c4ccccc4n5-c4ccc(-c5ccncc5)cc4)ccc32)cc1 ; c1ccc(-n2c3ccccc3c3cc(-c4ccc5c(c4)c4c6ccccc6ccc4n5-c4ccccc4)ccc32)cc1 ; c1ccc(-n2c3ccccc3c3cc(-c4ccc5c(c4)c4cc6ccccc6cc4n5-c4ccccc4)ccc32)cc1]

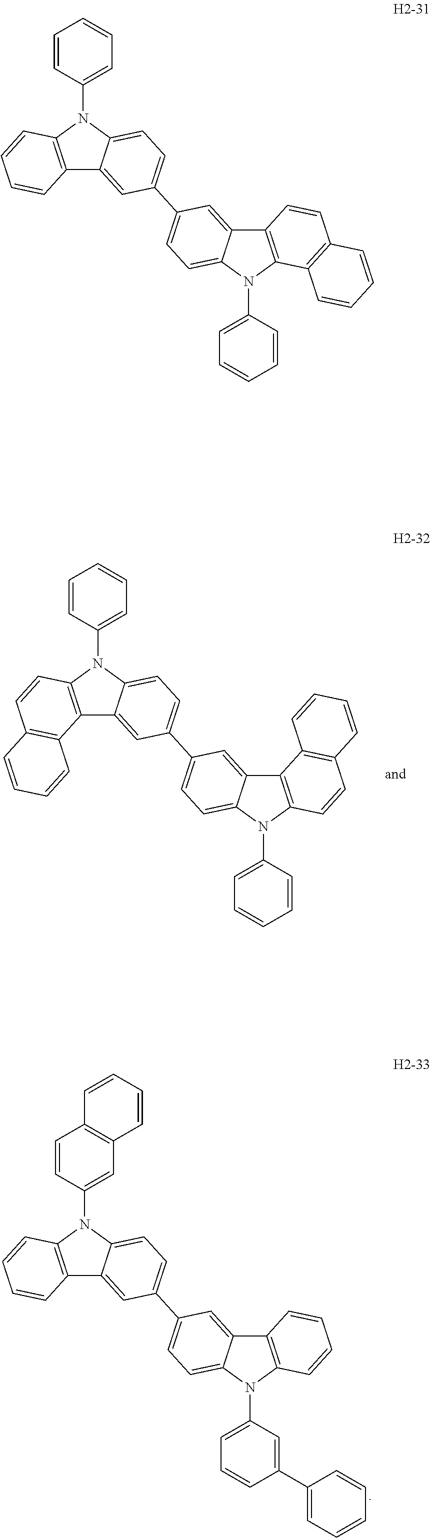 [SMILES: c1ccc(-n2c3ccccc3c3cc(-c4ccc5c(c4)c4ccc6ccccc6c4n5-c4ccccc4)ccc32)cc1 ; c1ccc(-n2c3ccc(-c4ccc5c(c4)c4c6ccccc6ccc4n5-c4ccccc4)cc3c3c4ccccc4ccc32)cc1 ; c1ccc(-c2cccc(-n3c4ccccc4c4cc(-c5ccc6c(c5)c5ccccc5n6-c5ccc6ccccc6c5)ccc43)c2)cc1]

10. An organic electroluminescent compound represented by the following formula 1: 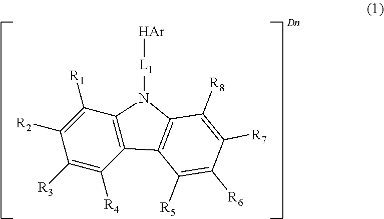
in formula 1,
HAr represents a substituted or unsubstituted (3- to 30-membered)heteroaryl;
L₁ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
R₁ to R₈, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NR₁₃R₁₄ or —SiR₁₅R₁₆R₁₇; or may be linked to an adjacent substituent to form a ring(s),
with the proviso that at least one group of group R₅ and R₆, group R₆ and R₇, and group R₇ and R₈ of formula 1 are fused with * of the following formula 1-a to form a ring(s), 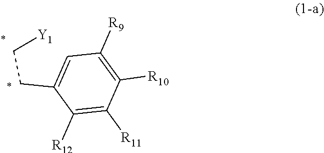
in formula 1-a,
Yl represents O or S,
R₉ to R₁₂, each independently, represent hydrogen, deuterium, a halogen, a cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NR₁₈R₁₉ or —SiR₂₀R₂₁R₂₂,
R₁₃ to R₂₂, each independently, represent hydrogen, deuterium, a halogen, a cyano, a substituted or unsubstituted (C1-C30)alkyl; a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl; or may be linked to an adjacent substituent to form a ring(s);
Dₙ represents that n hydrogens are replaced with deuterium; and
n represents an integer of 1 to 50.
